(19) United States
(12) Patent Application Publication
(10) Pub. No.: US 2011/0189194 A1
(43) Pub. Date: Aug. 4, 2011

(54) USE OF CD95 INHIBITORS FOR THE TREATMENT OF INFLAMMATORY DISORDERS

(75) Inventors: Ana Martin-Villalba, Heidelberg (DE); Elisabeth Letellier, Dippach (LU); Ignacio Sancho-Martinez, San Diego, CA (US)

(73) Assignee: Deutsches Krebsforschungszentrum Stiftung des offentlichen Rechts, Heidelberg (DE)

(21) Appl. No.: 13/054,269

(22) PCT Filed: Jul. 14, 2009
(86) PCT No.: PCT/EP2009/005127

§ 371 Date: Apr. 14, 2011

(30) Foreign Application Priority Data
Jul. 14, 2008 (EP) 08012685.7

(51) Int. Cl. A61K 39/395; A61P 29/00; A61P 25/00

(52) U.S. Cl. 424/158.1

(57) ABSTRACT

The present invention refers to the use of an inhibitor of the CD95/CD95L system for the prevention and/or treatment of an inflammatory disorder or for the prevention and/or treatment of an inflammatory process in a neuronal disorder, particularly in a CNS disorder.

The present invention refers to the use of an inhibitor of the CD95/CD95L system for the prevention and/or treatment of an inflammatory disorder or for the prevention and/or treatment of an inflammatory process in a neuronal disorder, particularly in a CNS disorder.

Death of neurons and oligodendrocytes is the ultimate cause of loss of function below the lesion site in spinal injured patients. Some of these cells actively switch on a death program for their own demise, apoptosis. The CD95Ligand (CD95L; FasUAPO1-L) is one of the best characterized triggers of apoptosis and its neutralization in spinal injured mice reduced the number of cells undergoing apoptosis. The achieved rescue of neurons and oligodendrocytes resulted in increased recovery of locomotor activity in the previously paralysed limbs. Improvement of motor function upon inhibition of CD95L was also observed in rats after contusion injury of the spinal cord and in spinal injured CD95-deficient MRU/pr mice (Ackery et al., Casha et al., Yoshino et al.). CD95L is a type II transmembrane protein poorly expressed in the naïve adult spinal cord. Upon injury it can be presented by resident spinal cord cells and infiltrating leukocytes. Identifying the source of detrimental-CD95L is crucial for the design of administration protocols for CD95L-neutralizing agents to treat spinal injuries.

There is increasing evidence that CD95L can be involved in processes other than apoptosis. In the CNS, we previously reported that CD95L increases the number of branches in developing neurons and the motility of malignant astrocytes (Kleber et al., 2008; Zuliani et al., 2006). Likewise, in dorsal root ganglion cells CD95L increases axonal growth (Desbarats et al., 2003). But also in the immune system CD95L can increase T cell proliferation (Kennedy et al., 1999).

CD95 (Fas, APO-1) has long been viewed as a death-inducing receptor. Triggering of CD95 by binding of its cognate ligand (CD95L, FasL, Apo-1L) leads to recruitment of the adaptor protein FADD to its death domain (DD) via homotypic interactions. Thereafter, interaction of the death-effector domain (DED) of FADD with procaspase-8 and -10 allows their recruitment and activation within the death-inducing signaling complex (DISC). These initiator caspases can activate downstream effector caspases finally committing the cell to death with or without further involvement of the mitochondrial pathway. However, the assumption of CD95 as an exclusive mediator of apoptosis has been put to rest. In the CNS, the CD95 system has been shown to increase branching of developing cells, axonal growth of dorsal root ganglion cells (DRGs) and increased migration of malignant glioma cells (Desbarats et al., 2003; Kleber et al., 2008; Zuliani et al., 2006). Whereas, in DRGs, the CD95 system is thought to mediate axonal growth via ERK activation, in malignant glioma cells, CD95 mediates migration via activation of the Src/PI3K/MMP pathway (Desbarats et al., 2003; Kleber et al., 2008). In the immune system, activation-induced cell death (AICD) was thoroughly described in activated cycling T-cells as a CD95-dependent process (Dhein et al., 1995; Krammer, 2000). In contrast, resting T-cells seem to be resistant to CD95-mediated apoptosis (Klas et al., 1993). However, further studies also showed a role for CD95L/CD95 in T cell proliferation by inducing the production of IL-2 (Kennedy et al., 1999). Solid evidence that the CD95L can also act as a proinflammatory mediator came from studies where tissue engineered to over-express CD95L was colonized by neutrophils (Kang et al., 1997; Seino et al., 1997). However, the molecular mechanism by which CD95 induces inflammation has remained elusive.

Injury to the spinal cord elicits an inflammatory response within the first hours after injury that lasts for several weeks. This response includes endothelial damage, release of proinflammatory mediators, changes in vascular permeability, infiltration of peripheral inflammatory cells and activation of astrocytes and microglia. Infiltrating inflammatory cells can on the one side promote wound healing events but, on the other side, release toxic factors that amplify tissue damage (Jones and Tuszynski, 2002; Rolls et al., 2009). Yet, the precise signals leading to leukocyte infiltration are still unknown.

Several studies illustrated an increased expression of CD95 in the injured spinal cord (Casha et al., 2001; Li et al., 2000; Matsushita et al., 2000; Sakurai et al., 1998; Zurita et al., 2001). Inhibition of CD95 signaling prevented death of motomeurons following spinal ischemia and axotomy of the facial nerve (Ugolini et al., 2003). Importantly, neutralization of CD95L significantly reduced death of neurons and oligodendrocytes and improved functional recovery of spinal injured animals (Demjen et al., 2004). These results were further confirmed in CD95-deficient mutant mice (lpr) (Casha et al., 2005; Yoshino et al., 2004) and in rats treated with a CD95-Fc (Ackery et al., 2006). However, the actual source of CD95L and the mechanism by which the CD95/CD95L system induces damage following injury had not been addressed yet.

According to the present invention, it was found that the CD95/CD95L system is involved in increasing migration of immune cells, particularly of neutrophils and/or macrophaages. Thus, inhibition of the CD95/CD95L system might be beneficial for the prevention and/or treatment of inflammatory disorders or for the prevention and/or treatment of inflammatory processes in neuronal disorders. The present invention is particularly suitable for use in human medicine.

A first aspect of the present invention refers to the treatment of inflammatory disorders. Specific examples of inflammatory disorders are chronic inflammatory bowel disease, e.g. Morbus Crohn or colitis ulcerosa, inflammatory rheumatoid disorders associated with increased macrophage activity, e.g. rheumatoid arthritis, chronic polyarthritis, ankylosating spondylitis (Morbus Bechterew), psoriatic arthritis, juvenile idiopathic arthritis as well as collagenoses, i.e. connective tissue disorders and vasculitides, i.e. inflammatory vasculatory disorders such as lupus erythematodes, sclerodermia, Sjögren-syndrome, polymyositis and dermatomyositis, mixed collagenose and Wegener-granulomatosis (Morbus Wegener).

In this embodiment of the invention, a CD95/CD95L inhibitor may be administered in a therapeutically effective dose and by a route suitable for the treatment of the above disorders. The administration may e.g. be locally or systemically, preferably by injection or infusion or by any other suitable route.

A second aspect of the present invention refers to the treatment of inflammatory processes in neuronal disorders. Specific examples of neuronal disorders are CNS disorders, such as cerebral or spinal cord injury, e.g. cerebral lesions or partial or complete spinal core lesions, e.g. stroke, particularly paraplegia. Although the use of CD95/CD95L inhibitors for the treatment of CNS disorders is already disclosed in WO 2004/071528, the present invention differs therefrom by referring to the prevention and/or treatment of inflammatory processes in such a disorder. Since inflammatory processes in CNS disorders are associated with migration of immune cells, e.g. neutrophils, the inhibitor is administered in a therapeutically effective dose and by a route to reduce or inhibit immune cell, e.g. neutrophil and/or macrophage migration. Preferably, the inhibitor is administered immediately after occurrence of CNS injury, e.g. immediately after the occurrence of the injury, e.g. up to 2 h, 4 h, 6 h or 8 h after the occurrence of the injury. Further, it is preferred that the composition is systemically administered, thereby reducing the activity of immune cells in the whole organism to be treated.

In a preferred embodiment of the invention, the inhibitor is a CD95-ligand (Fas ligand; APO1 ligand) inhibitor. For example, CD95-ligand inhibitors may be selected from
(a) an inhibitory anti-CD95 ligand-antibody or a fragment thereof;
(b) a soluble CD95 receptor molecule or a CD95 ligand-binding portion thereof; and
(c) a Fas ligand inhibitor selected from FLINT, DcR3 or fragments thereof.

Preferred are inhibitory anti-CD95L-antibodies and antigen-binding fragments thereof and soluble CD95R molecules or CD95L-binding portions thereof. Examples of suitable inhibitory anti-CD95L antibodies are disclosed in EP-A-0 842 948, WO 96/29350, WO 95/13293 or as well as chimeric or humanized antibodies obtained therefrom, cf. e.g. WO 98/10070. Further preferred are soluble CD95 receptor molecules, e.g. a soluble CD95 receptor molecule without transmembrane domain as described in EP-A-0 595 659 and EP-A-0 965 637 or CD95R peptides as described in WO 99/65935, which are herein incorporated by reference.

Especially preferred is a CD95L inhibitor which comprises an extracellular domain of the CD95R molecule (particularly amino acids 1 to 172 (MLG . . . SRS) of the mature CD95 sequence according to U.S. Pat. No. 5,891,434) optionally fused to a heterologous polypeptide domain, particularly a Fc immunoglobulin molecule including the hinge region e.g. from the human IgG1 molecule. Particularly preferred fusion proteins comprising an extracellular CD95 domain and a human Fc domain are described in WO 95/27735 and PCT/EP2004/003239, which are herein incorporated by reference.

Further preferred inhibitors are multimeric CD95R fusion polypeptides comprising the CD95R extracellular domain or a fragment thereof and a multimerization domain, particularly a trimerization domain, e.g. bacteriophage T4 or RB69 foldon fusion polypeptides as described in WO 2008/025516, which is herein incorporated by reference.

The Fas ligand inhibitor FLINT or DcR3 or a fragment, e.g. a soluble fragment thereof, for example the extracellular domain optionally fused to a heterologous polypeptide, particularly a Fc immunoglobulin molecule is described in WO 99/14330, WO 99/50413 or Wroblewski et al., Biochem. Pharmacol. 65, 657-667 (2003), which are herein incorporated by reference. FLINT and DcR3 are proteins which are capable of binding the CD95 ligand and LIGHT, another member of the TNF family.

In a further embodiment of the present invention, the inhibitor is a CD95R inhibitor which may be selected from
(a) an inhibitory anti-CD95 receptor-antibody or a fragment thereof; and
(b) an inhibitory CD95 ligand fragment.

Examples of suitable inhibitory anti-CD95R-antibodies and inhibitory CD95L fragments are described in EP-A-0 842 948 and EP-A-0 862 919 which are herein incorporated by reference.

In still a further embodiment of the present invention the inhibitor is a nucleic acid effector molecule. The nucleic acid effector molecule may be selected from antisense molecules, RNAi molecules and ribozymes which are capable of inhibiting the expression of the CD95R and/or CD95L gene.

In a still further embodiment the inhibitor may be directed against the intracellular CD95R signal transduction. Examples of such inhibitors are described in WO 95/27735 e.g. an inhibitor of the interleukin 1β converting enzyme (ICE), particularly 3,4-dichloroisocoumarin, YVAD-CHO, an ICE-specific tetrapeptide, CrmA or usurpin (WO 00/03023). Further, nucleic acid effector molecules directed against ICE may be used.

In still a further embodiment, the inhibitor may be directed against a metalloproteinase (MMP), particularly against MMP-2 and/or MMP-9.

The inhibitor or a combination of the above inhibitors is administered to a subject in need thereof, particularly a human patient, in a sufficient dose for the treatment of the specific condition by suitable means. For example, the inhibitor may be formulated as a pharmaceutical composition together with pharmaceutically acceptable carriers, diluents and/or adjuvants. Therapeutic efficacy and toxicity may be determined according to standard protocols. The pharmaceutical composition may be administered systemically, e.g. intraperitoneally, or intravenously, or locally, e.g. intrathecally or by lumbar puncture.

The dose of the inhibitor administered will of course, be dependent on the subject to be treated, on the subject's weight, the type and severity of the injury, the manner of administration and the judgement of the prescribing physician. For the administration of anti-CD95R or L-antibodies or soluble CD95R proteins, e.g. CD95-Fc fusion proteins, a daily dose of 0.001 to 100 mg/kg is suitable.

Further, the present invention is explained in more detail by the following Figures and Examples.

### DESCRIPTION OF THE FIGURES

FIG. 1: Alignment of the T-4 and RB69-Foldon sequence

Alignment of the C-terminal sequences of bacteriophage T4 and bacteriophage RB69 fibritin (accession numbers CAA31379 and NP-861864). Identical amino acid residues are marked.

FIG. 2: Sequence of the CD95-RB69 fusion protein

The amino acid sequence of the CD95-RB69 fusion protein is shown. The endogenous CD95 signal-peptide is underlined, and the CD95-ECD is printed in bold letters; whereas the RB69 fibritin-Foldon sequence is printed in red letters. The linker between the CD95-ECD as well as the flexible positioned strep-tag-II is printed in blue letters. Please note, that R17 is the first amino-acid of the secreted protein (marked by an additional number 1 in bold face) and that the R87S mutation refers to this enumeration. Arginine 87 is printed in bold-face and underlined.

FIG. 3: SEC-analysis of affinity purified CD95-RB69 fusion proteins

After affinity purification, approximately 100 μg of CD95-RB69 (A) or CD95(R87S)-RB69 (B) in a final volume of 0.1 ml were separated on a Superdex200 10-300GL column (GE Healthcare, Germany) at a flow rate of 0.5 ml/min using PBS as running buffer. The CD95-RB69 fusion proteins elute within a symmetrical, well shaped peak from the column. Based on the calibration of the SEC-column, the peaks eluting after 11.21 (A) or 10.93 ml (B) correspond to apparent molecular weights of approx. 240 and 280 kDa.

FIG. 4: SDS-PAGE analysis (silver-stain) of SEC fractions from affinity purified CD95-RB69 fusion proteins

SEC fractions A1-A14 (lane numbers 1 to 14; M=marker) of the CD95-RB69 (A) or CD95(R87S)-RB69 (B) elution profile were analysed by SDS-PAGE (silver-stain), performed under reducing conditions. A major protein band running between 30-40 kDa is detected in the peak fractions; shown by an arrowhead.

FIG. 5: Effect of CD95-RB69 or CD95(R87S)-RB69 on the induction of apoptosis by human (A) or mouse (B) CD95L-T4 on human Jurkat cells.

Mutation of R87S abrogates the ability of the CD95-RB69 protein to inhibit CD95L-mediated killing of Jurkat cells. Jurkat cells were incubated with 250 ng/ml of human (A) or mouse (B) CD95L-T4 in the presence of wild-type and mutant CD95-RB69 in duplicates for each concentration of the fusion proteins. Decreased cell death is represented by low DEVD-AFC cleavage rates.

FIG. 6: CD95L induces migration of neutrophils and macrophages through activation of PI3K/β-catenin/MMP signalling.

A In a two chamber in vitro migration assay, CD95L-T4 induced migration of neutrophils. Data are representative of at least 3 independent experiments.

B CD95L-T4 induced MMP-9 expression in neutrophils. Data are representative of at least 2 independent experiments. C MMP-2/9 inhibitor blocked CD95L-T4 induced migration of neutrophils. D CD95L-T4 induced in vitro migration of macrophages. Data are representative of 5 independent experiments. E Neutralizing antibodies to CD95L (MFL3) blocked basal migration of macrophages. Data from 2 independent experiments were pooled and represented as % of migrating cells.

FIG. 7. Increased cell surface expression of CD95L on mouse and human myeloid cells after SCI. (A) Experimental setup for eGFP bone marrow chimeras. (B) Time kinetics of infiltrating immune cells into the injured spinal cord 1 to 14 days after SCI in bone marrow chimeras from eGFP-donor mice and lethally irradiated wt recipient mice (BMT-eGFP). (C) Immune cell type present at the lesion site 24 h after SCI. (D) Constitutive expression of CD95L on peripheral blood neutrophils and monocytes and its increase after SCI. n=4/group; *p<0.05, **p<0.01. Data are representative of at least 2 independent experiments (E) Representative histogram of CD95L surface expression on neutrophils from a spinal cord (SC)-injured patient (first and last time point after injury from patient d are presented) or a healthy control. (F) Quantification of CD95L expression on neutrophils from 5 SC-injured patients and 3 patients with spinal disc herniation relative to levels in respective controls. A: first time point at admission at the hospital after the injury varying between 2 hours and 5 hours after injury. d:days after injury. Data are presented as mean±SEM; CD95L expression on SC-injured patient's blood is representative of at least 3 independent stainings.

FIG. 8. Syk kinase activation in myeloid cells leads to PI3K activation upon CD95 stimulation. (A,B) CD95L-T4 (Kleber et al., 2008) induced phosphorylation of AKT in neutrophils (A) and macrophages (B). (B) CD95L-T4 induced phosphorylation of Src in primary macrophages upon CD95 stimulation. tAKT: total AKT, pAKT: phosphorylated AKT, tSrc: total Src, pSrc: phosphorylated Src, (C) Experimental layout for SH2 arrays: detection of CD95 that itself or through an adaptor within a protein complex is bound to an SH2-containing protein via a phosphorylated tyrosine (pY) in CD95L-stimulated bone-marrow derived neutrophils or in vivo activated neutrophils from the peritoneum of thioglycolate-injected mice. (D,E) Peptide receptor competition experiments in dHL-60 (D) and primary macrophages (E). Syk kinase binds to a phosphorylated but neither to an unphosphorylated sequence of CD95 nor to a scramble phosphorylated peptide. (F) Phosphorylation of Syk kinase in primary macrophages upon CD95 stimulation. pSyk: phosphorylated Syk, tSyk: total Syk. (G,H) Knockdown of Syk kinase abolished CD95L-induced phosphorylation of AKT (G, right panel: efficient knockdown of Syk) and Src (H) in primary macrophages. All data are representative of at least 3 independent experiments.

FIG. 9. CD95L stimulation triggers migration of myeloid cells through activation of MMP's via Syk kinase. (A-C) Experimental layout for assessment of migration and MMP activity. (D-F) In a two chamber in vitro migration assay, CD95L-T4 induced migration of primary neutrophils (D), dHL-60 (E) and primary macrophages (F). (G-I) CD95L-T4 induced MMP-9 activation in neutrophils (G), dHL-60 (H) and primary macrophages (I). (J-L) MMP-2/9 inhibitor blocked CD95L-T4 induced migration of neutrophils (J), dHL-60 (K) and macrophages (L). (M) Neutralizing antibodies to CD95L (MFL3) blocked basal migration of macrophages. (N,O) Syk knockdown reduced CD95L-induced migration of dHL-60 (N) and macrophages (0). (P,Q) Efficient knockdown of Syk in dHL-60 (P) and macrophages (Q). (R) Syk knockdown abolished CD95L-T4 induced MMP-9 activation in macrophages. (S) Scheme representing the signalling pathway of CD95L-induced migration. All data are representative of at least 3 independent experiments with at least 6 technical replicates per condition for migration assays. Data are presented as mean±SEM; *p<0.05; **p<0.01.

FIG. 10. CD95L on myeloid cells is involved in self-recruitment to the site of injury in vivo. (A) Phosphorylation of AKT in peripheral blood cells was assessed by flow cytometry. Injury to the spinal cord increased % of pAKT positive cells in wt mice but not in CD95L′ mice. n=4-5/group; *p<0.05. (B) Experimental layout for assessing the infiltration of immune cells to the spinal cord after SCI. (C) Infiltration of immune cells, especially neutrophils (CD45:GR-1^{high}) was reduced in the injured spinal cord of CD95L^{f/f;LysMcre} mice at 6 h (n=4/group; *p<0.05; **p<0.01) and 24 h (n=4/group; *p<0.05; **p<0.01) or in mice acutely treated with CD95L-neutralizing CD95-trimer (CD95-RB69) at 24 h (n=3-5/group; **p<0.01) after SCI compared to their respective controls. (D) Reduced infiltration of macrophages (CD45: CD11b⁺, F4/80+) in the injured spinal cord of CD95L^{f/f;LysMcre} mice (n=3/group; *p<0.05) at 7 days following SCI. (E) Experimental layout for assessing the infiltration of immune cells to the peritoneum after thioglycolate-induced peritonitis. (F) Reduced infiltration of neutrophils in a thioglycolate-induced peritonitis model in CD95L^{f/f;LysMcre} mice (n=6/group; **p<0.01) or mice treated acutely with CD95-RB69 (n=4/group; *p<0.05) compared to their respective controls. (G) Reduced infiltration of macrophages in the peritoneum of CD95L^{f/f;LysMcre} mice after thioglycolate injection (n=3-5/group; *p<0.05). (G) Reduced infiltration of macrophages in the peritoneum of lpr mice after thioglycolate injection (n=6/group; *p<0.05).

FIG. 11. Deletion of CD95L in myeloid cells improves functional recovery of spinal injured mice. (A,B) 24 h after transection injury, CD95L^{f/f;LysMcre} mice exhibited lower levels of CD95L mRNA (n=6/group; **p<0.01) (A) and of caspase-3 activity (n=4/group; ***p<0.001) (B) compared to control littermates. mRNA levels were normalized to naïve wt animals. (C) 10-11 weeks after crush injury, CD95L^{f/f;LysMcre} mice exhibited increased number of NeuN⁺ cells (NeuN: a marker for mature neurons) compared to CD95L^{f/f} control littermates (n=6/group; **p<0.01). (D) 10-11 weeks after crush injury, improved white matter sparing; as determined by the distance between the lost CNPase signal rostral and the reappearance of the CNPase staining caudal to the lesion site in the dorsal funiculus of the spinal cord, was observed in CD95L^{f/f;LysMcre} mice as compared to the respective control littermates. (n=6/group; **p<0.01) (E) In CD95L^{f/f;LysMcre} overall improvement was achieved compared to CD95L^{+/f LysMcre} and CD95L^{f/f} control littermates in the BMS (to CD95L^{f/f}: p<0.05; to CD95L^{+/f LysMcre}: p<0.01; Koziol test, n=10-12/group) as well as in the swimming test (compared to CD95L^{f/f}: p<0.05; compared to CD95L^{+/f LysMcre}: p<0.01; Koziol test, n=10-12/group) after transection injury of the spinal cord. As littermate controls (CD95L^{f/f} and CD95L^{f/+;LysMcre) showed no significant difference in locomotor activity, their results were pooled in the crush injury model. (F) In a crush injury model of the spinal cord, CD}95L^{f/f;LysMcre} mice achieved overall improvement compared to control littermates in the BMS (p<0.01; Koziol test, n=10-11/group) as well as in the swimming test (p<0.01; Koziol test, n=10-11/group). Data are presented as mean±SEM.

FIG. 12. Deletion of CD95L in myeloid cells regulates the inflammatory environment following SCI. (A,B) Gene expression profiling was assessed in CD95L^{f/f;LysMcre} mice and CD95-RB69 treated mice and their respective controls 24 h after SCI (n=3/group). Functional overrepresentation of the significant regulated genes at 5% false discovery rate (FDR) in CD95L^{f/f;LysMcre} mice and CD95-RB69 treated mice. Two clusters of transcripts showing down-regulation of genes involved in apoptosis and immune response in CD95L^{f/f;LysMcre} compared to CD95L^{f/f} control mice (A) and in CD95-RB69 compared to vehicle-treated animals 24 h after SCI (B). The color codes are green for down-regulated genes, red for up-regulated genes and black for no changes as compared to their injured respective controls. (C) 65.2% of genes were commonly regulated on the dataset of CD95L^{f/f;LysMcre} compared to CD95L^{f/f} littermates and CD95-RB69-treated compared to vehicle-treated mice 24 h after SCI. (D) Validation of microarray data by qRT-PCR: mRNA levels of CXCL10, IL-1β, IL-6, CCL6 and Stat-3 24 h after SCI. (n=4/group; *p<0.05; **p<0.01) (E) Identification of a common gene signature in the injured spinal cord independent of the site of CD95L inhibition. Three different datasets were analysed for gene expression profiling 24 h after SCI: (1) CD95L^{f/f;LysMcre}: mice with deletion of CD95L in myeloid cells; (2) CD95-RB69-treated: CD95L was pharmacologically inhibited after injury; (3) CD95L′ mice with ubiquitous deletion of CD95L, and their respective counterparts. Upon meta-analysis we found 612 genes common to all three datasets at 5% false discovery rate (FDR). Hierarchical clustering of these 612 differentially regulated genes upon SCI in all three datasets. The color codes are green for down-regulated genes, red for up-regulated genes and black for no changes. (F) Caspase-3 activity 7 days following SCI in CD95^{f/f;LysMcre} and their respective littermate controls CD95^{f/f}. (n=4-5/group; *p<0.05; **p<0.01). Data are presented as mean±SEM. ns: not significant.

FIG. 13. CD95L expression levels and apoptosis levels following SCI. (A) Time kinetics of CD95L mRNA levels after SCI. CD95L mRNA levels peaked 24 h after SCI. (B) Time kinetics of caspase-3 activity after SCI. Caspase-3 activity was significantly increased at 7 and 10 days after injury and returned to levels of control animals at 14 days. Data are presented as mean±SEM; *p<0.05; **p<0.01; ***p<0.001 (n=3-4/group).

FIG. 14. FADD is not recruited to the CD95 DISC upon CD95 stimulation in primary macrophages. No FADD recruitment to the CD95 DISC upon CD95 stimulation in primary macrophages as compared to CD95L-sensitive mouse thymoma cells (E20) used as a positive control. Data are representative of at least 2 independent experiments.

FIG. 15. Activation of Src in dHL-60 and effect of Src inhibition in dHL-60 and primary macrophages upon CD95 stimulation. (A) Src phosphorylation in dHL-60 upon CD95 stimulation. Data are representative of at least 4 independent experiments. (B,C) CD95L-induced Syk activation is inhibited after PP2 treatment in dHL-60 (B, upper panel (CD95L, 20 ng/ml) and lower panel (CD95L, 40 ng/ml)) and in primary macrophages (C). Data are representative of at least 2 independent experiments.

FIG. 16. Characterization of CD95L^{f/f;LysMcre} mice. (A) Successful recombination of cre in CD95L^{f/f;LysMcre} mice. Bone marrow CD11b⁺ cells were positively sorted by beads and CD95L mRNA levels were analyzed in CD95L^{f/f;LysMcre} and respective control littermates. CD95L mRNA was reduced by 2.2 fold in CD95L^{f/f;LysMcre} compared to control animals. (B) CD95L mRNA levels were analyzed in thioglycollate-elicited neutrophils 6 h after injection in CD95L^{f/f;LysMcre} and their respective controls. mRNA levels of CD95L were highly down-regulated in CD95L^{f/f;LysMcre} compared to control littermates. (C) CD95L mRNA levels were analyzed in thioglycollate-elicited macrophages 72 h after injection in CD95L^{f/f;LysMcre} and their respective controls. mRNA levels of CD95L were highly down-regulated in CD95L^{f/f;LysMcre} compared to the control littermates. (D,E) Percentage of blood CD11b⁺ cells, neutrophils, monocytes, B and T cells was analyzed by their appropriate cell markers. No difference in blood cell population was observed between uninjured or injured CD95L^{f/f;LysMcre} compared to uninjured or injured control littermates CD95L^{f/f} and CD95L^{f/+;LysMcre}, respectively. (F) Absolute numbers of blood cells were not significantly changed in CD95L^{f/f;LysMcre} compared to control littermates. Data are presented as mean±SEM; *p<0.05; **p<0.01, ***p<0.001. (G) CD95L-induced migration is independent of cytokine production. Cytokine mRNA levels were analyzed in thioglycollate-elicited cells from CD95L^{f/f;LysMcre} and their respective controls 6 h after thiogylcollate injection. mRNA levels of CXCL10, IL-1, IL-6 and CXCL2 were not changed in CD95L^{f/f;LysMcre} compared to control animals. Data are presented as mean±SEM; *p<0.05; **p<0.01.

FIG. 17. Number of neutrophils undergoing apoptosis in mice lacking

CD95L activity and their respective controls (A) Annexin V staining of neutrophils in the spinal cord 24 h after injury in animals treated with CD95-RB69 or CD95-(R87S)-RB69. (B) Annexin V staining in thioglycollate-elicited neutrophils 6 h after injection in CD95L^{f/f;LysMcre} and respective control animals. (C) Annexin V staining in thioglycollate-elicited neutrophils 6 h after injection in animals treated with CD95-RB69 or CD95-(R87S)-RB69. Data are presented as mean±SEM.

FIG. 18. Deletion of CD95L in immune cells improves functional recovery and reduces apoptosis of spinal resident cells. (A) Experimental setup. (B,C) 24 h after transection injury, BMT-CD95L^{-/-} chimeras exhibited lower levels of CD95L mRNA (B) and of caspase-3 activity (C) compared to BMT-wt controls. mRNA levels were normalized to naïve wt animals. (D) 10-11 weeks after crush injury, BMT-CD95L^{-/-} chimeras exhibited increased number of NeuN⁺ cells compared to BMT-wt chimeras. (E) Oligodendrocyte survival was analysed by determining the distance between the lost CNPase signal rostral and the reappearance of the CNPase staining caudal to the lesion site in the dorsal funiculus of the spinal cord. 10-11 weeks after crush injury BMT-CD95L^{-/-} chimeras exhibited a shorter distance as compared to the respective controls, indicating increased white matter sparing in BMT-CD95L^{-/-} chimeras. (F-G) In a transection model (G) or a crush injury model (F) of the spinal cord, BMT-CD95L^{-/-} mice achieved overall improvement in the BMS as well as in the swimming test compared to BMT-wt chimeras (transection injury: BMS p<0.01, n=12-13/group; Swimming test p<0.001, n=12-13/group) (crush injury: BMS p<0.05, n=8/group; Swimming test p<0.05, n=8/group). Data are presented as mean±SEM; *p<0.05; **p<0.01, ***p<0.001.

FIG. 19. Deletion of CD95L on T cells does not promote functional recovery in spinal injured mice. (A) Cre recombination in CD95L^{f/f;LysMcre} animals was assessed by cre staining in blood T cells. (B) In a crush injury model of the spinal cord, functional recovery was assessed by using the BMS as well as the swimming test in CD95L^{f/f;LCKcre} and their respective control littermates, CD95L^{f/f} and CD95L^{f/+;LCKcre}. No difference in functional improvement was observed in CD95L^{f/f;LysMcre} compared to their control littermates in both ratings (n=10-12/group). Data are presented as mean±SEM.

FIG. 20. Microarray functional overrepresentation of the CD95L^{f/f;LysMcre} mice dataset and of the 612 significantly differentially regulated genes in all datasets studied. (A) Gene expression profiling was assessed in CD95L^{f/f;LysMcre} mice and their respective littermate controls 24 h after SCI. Functional overrepresentation of the significant regulated genes at 5% false discovery rate (FDR) in CD95L^{f/f;LysMcre} mice. (B) Functional overrepresentation of the significantly differentially regulated genes in all three datasets analyzed: (1) CD95L^{f/f;LysMcre}: mice with deletion of CD95L in myeloid cells; (2) CD95-RB69-treated: CD95L was pharmacologically inhibited after injury; (3) CD95L^{-/-} mice with ubiquitous deletion of CD95L, and their respective counterparts.

FIG. 21. CD95 mRNA levels in CD95^{f/} and CD95^{f/f;Nescre} mice. Cre recombination led to reduced amounts of CD95 mRNA levels in the spinal cord of CD95^{f/f;Nescre} mice.

FIG. 22: List of the 612 genes that were consistently and significantly differentially regulated in the injured spinal cord 24 hours after SCI in all three datasets analyzed.

### EXAMPLES

### 1. CD95L-Induced Migration of Neutrophils and Macrophages

### 1.1. Material and Methods

### 1.1.1. Cell Isolation of Murine Neutrophils and Culture

Bone marrow neutrophils were isolated from the femur of mice by flushing the bones with PBS/2 mM EDTA. Harvested bone marrow cells were resuspended in ACK buffer (150 mM NH₄Cl, 10 mM KHCO₃, 1 mM Na₂EDTA, pH 7.3) and incubated for 1 min to lyse erythrocytes. Neutrophil selection was performed using MACS-positive selection by magnetic beads according to the manufacturer's protocol (Miltenyi, #130-092-332). Purity of neutrophils was assessed by FACS and reached >96%.

In vivo activated neutrophils were isolated by washing the peritoneal cavity of mice 6 h after the injection of 3% thioglycollate.

1.1.2. Cell Isolation of CD11b+ Cells

Bone marrow cells were isolated as previously described. CD11b selection was performed according to the manufacturer's protocol (Miltenyi #130-092-333).

### 1.1.3. Primary Cell Culture

To obtain bone marrow-derived macrophages (BMDM), femurs and tibias were harvested bilaterally and marrow cores were flushed using syringes filled with PBS/2 mM EDTA. Cells were triturated and RBCs were lysed (0.15 mol/L NH₄CI, 10 mmol/L KHCO₃, 0.1 mmol/L Na₂EDTA; pH 7.4). After washing once in media, the cells were plated and cultured in RPMI 1640 supplemented with 1% penicillin/streptomycin, 0.001% a-mercaptoethanol, 10% FBS, 1% L-glutamine, 1% non essential amino-acids, 1% sodium pyruvate and 20% supernatant from macrophage colony stimulating factor secreting L929 cells. The sL929 drives bone marrow cells towards a macrophage phenotype (7-10 days). At day 1 non-adherent cells were collected and further cultivated. 4 days later fresh medium was added to boost the cell growth. At harvest, 95±0.7% of cells were macrophages (assessed by CD11b and F480 immunostaining). Supplemented culture media was replaced with RPMI/10% FBS on the day of stimulation so that stimulations were performed in the same media for all cell types.

### 1.1.4. Immunoprecipitation

At least 1×10⁷ cells were treated with 10 (neutrophils) or 20 (macrophages) ng/ml of mCD95L-T4 for 5 minutes at 37° C. or left untreated, washed twice in PBS plus phosphatase inhibitors (NaF, NaN₃, pNPP, NaPPi, β-Glycerolphosphate, 10 mM each and 1 mM orthovanadate), and subsequently lysed in buffer A [(20 mM Tris/HCl, pH 7.5, 150 mM NaCl, 2 mM EDTA, 1 mM phenylmethylsulfonyl fluoride, protease inhibitor cocktail (Roche), 1% Triton X-100 (Serva, Heidelberg, Germany), 10% glycerol, and phosphatase inhibitors (NaF, NaN3, pNPP, NaPPi, β-Glycerolphosphate, 10 mM each and 1 mM orthovanadate)]. Protein concentration was determined using BCA kit (Pierce). 500 μg of protein was immunoprecipitated overnight with either 5 μg anti-CD95 Ab Jo2 (BD #554255) and 40 μl protein-A Sepharose or the corresponding isotype control (BD #554709). Beads were washed 5 times with 20 volumes of lysis buffer. The immunoprecipitates were mixed with 50 μl of 2× Laemmli buffer and analyzed on 15% SDS-PAGE. Subsequently, the gels were transferred to Hybond nitrocellulose membrane (Amersham Pharmacia Biotech, Freiburg, Germany), blocked with 5% milk in PBS/Tween (PBS plus 0.05% Tween 20) for 1 hour, and incubated with the primary antibody in 5% milk in PBS/Tween at 4° C. overnight. Blots were developed with a chemoluminescence method following the manufacturer's protocol (PerkinElmer Life Sciences, Rodgan, Germany). The highly CD95L sensitive thymoma cells (E20) were included as a positive control for analysing FADD recruitment (anti-FADD mouse monoclonal Ab, clone 1F7, Millipore #05-486)

### 1.1.5. Western Blots

Protein extraction and immunoblotting was performed as previously described. Membranes were probed with the following antibodies: phosphorylated AKT (P-Ser473-AKT, 1:1000, Cell signalling #9271), total AKT (T-AKT, 1:1000, Cell Signaling #9272).

### 1.1.6. Migration Assay

Migration of bone marrow derived neutrophils or macrophages was assessed in vitro in a two chamber migration assay. Transwell inserts [3 μm (BD #353096) or 8 μm (BD #353097) pore size for neutrophils or macrophages respectively] were coated with matrigel (50 μg/ml; BD #354234). 5×10⁵ neutrophils or macrophages were plated in 500 μl medium onto the upper chamber. Cells were left untreated or treated with CD95L-T4 by adding 10, 20 and 40 ng/ml to the upper chamber. The number of migrated cells was counted 3 hours for neutrophils and 24 hours for macrophages after treatment. CD95L-induced migration of macrophages was analysed by blocking basal migration of macrophages by using neutralizing antibodies to CD95L (MFL3, 10 μg; BD #555290) or the appropriate isotype control (IgG, 10 μg; BD #554709).

The role of metalloproteinases on neutrophil recruitment was investigated by using selective inhibitors of MMP-2/9. Neutrophils were pre-incubated with MMP-2/9 inhibitor (50 μM; Calbiochem #444251) 30 minutes prior to CD95L-T4 treatment and migrating cells were calculated.

### 1.1.7. Gelatin Zymography for Activated MMPs

MMP activity in cell-free supernatants from neutrophils treated with different doses of CD95L-T4 was determined by gelatinase zymography as described previously. In brief, neutrophils were treated with CD95L-T4 (10 and 20 ng/ml) for 6 hours. After electrophoresis and washing the gel with Triton X-100 (2.5% v/v, twice for 30 min), the gel was incubated in MMP reaction buffer [50 mmol/L Tris-HCl (pH 7.8), 200 mmol/L NaCl, 5 mmol/L CaCl₂] at 37° C. for 16 h. Gelatinolytic activity was detected as transparent bands on staining with Coomassie Brilliant Blue G-250 solution and incubation in destaining solution (10% acetic acid, 20% methanol).

### 1.1.8. Engineering and Characterisation of Trimeric CD95-Fusion Proteins CD95-RB69 and CD95(R87S)-RB69

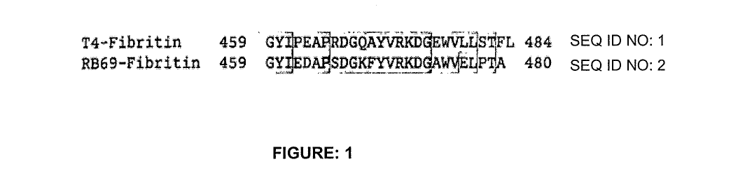

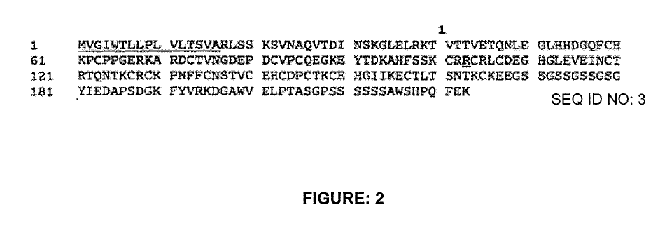

For the analysis of the of CD95/CD95L-interaction, the extracellular domain of CD95 is commonly used in form of recombinant dimeric fusion proteins. Currently, all commercially available recombinant CD95 proteins exhibit a C-terminally fused Fc-part of human or mouse IgG1 (CD95-Fc), e.g. as described in WO 2004/085478, which is herein incorporated by reference. To avoid Fc-based effector functions interfering with the readout strategy of this study, we therefore decided to design a CD95L-Trap based on an different protein scaffold. Due to the proposed three receptor binding sites per CD95L-trimere, a trimeric CD95-fusion protein should be the ideal CD95-ligand-trap. We used a homologue of the T4-Foldon, derived from bacteriophage RB69 (FIGS. 1 and 2). This construct is described in WO 2008/025516, which is herein incorporated by reference. To ensure its specificity, a mutein of the designed CD95L-trap with an single amino-acid exchange in the CD95-ECD (Arg87Ser) was expressed and used as control within the described experiments. This single amino-acid exchange is known to abrogate the binding of human CD95 to human CD95L (Starling et al., 1997).

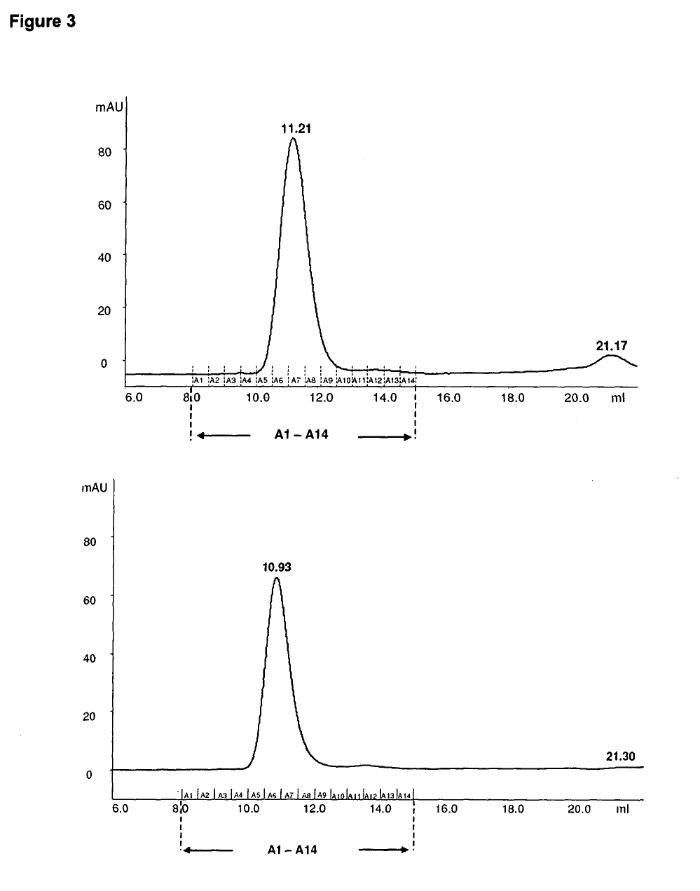

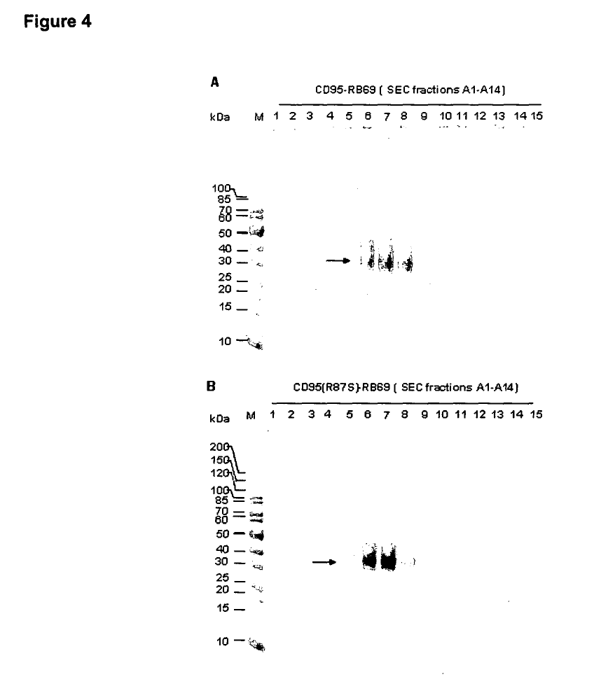

Indeed, secretory based expression of the CD95- or CD95(R87S)-RB69-Foldon fusion proteins resulted in the formation of a glycosylated, stable protein species. (FIGS. 3 and 4).

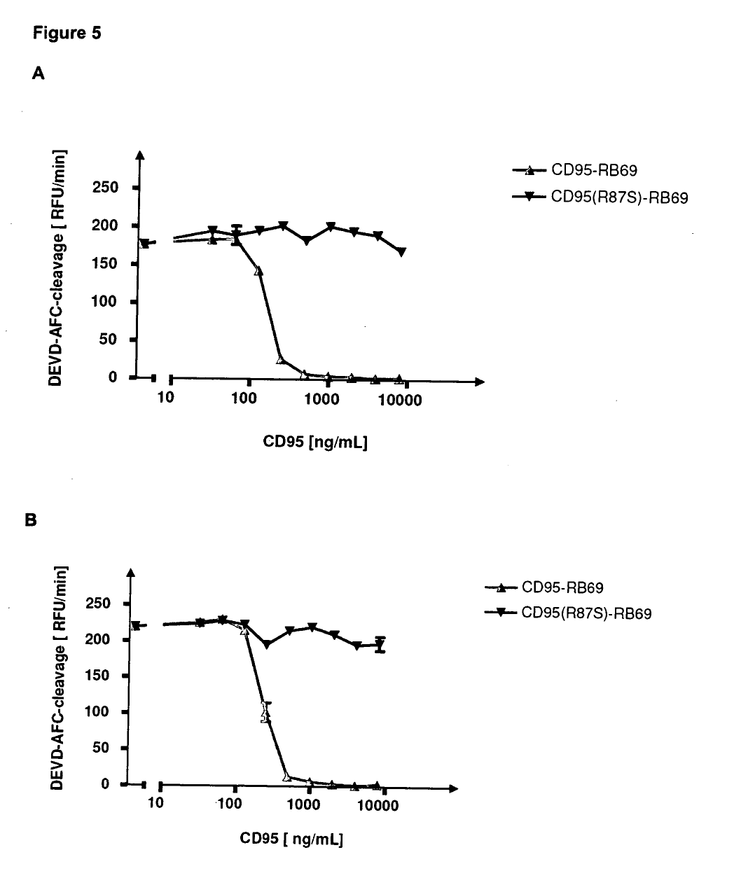

While we were using a human CD95 fusion protein in mouse, we had to analyse the binding of the R87S based control-protein for the human CD95/murine CD95L-interaction prior to the studies performed. We addressed this question by examining the ability of the CD95-fusion proteins to neutralise the apoptosis inducing capacity of either human or mouse CD95L on Jurkat cells in vitro. Whereas the human CD95-RB69 protein efficiently neutralises the apoptotic activity of human and mouse ligand in vitro, the R87S-control protein has no protective effect (FIG. 5 and FIG. 17E).

### 1.1.9. Protein Design

The RB69 derived fibritin foldon domain was fused C-terminally to the human CD95-ECD (M1-E168). Between the CD95-ECD and the RB69-Foldon (Tyr181-Ala205), a flexible linker element (Gly169-Ser180) was placed. For purification and analytical strategies, a streptag-II including a flexible linker element (Ser206-Lys223) was added C-terminally. The amino acid sequence of the fusion protein was backtranslated and its codon usage optimised for expression in mammalian cells. Gene synthesis was done by ENTELECHON GmbH (Regensburg, Germany). In the case of the CD95(R87S)-RB69-protein, the necessary codon exchange in the expression cassette was introduced by PCR-based mutagenesis. The sequence-verified expression cassettes were subcloned into pCDNA4-HisMax-backbone, using unique Hind-III- and Not-I-sites of the plasmid.

### 1.2. Results

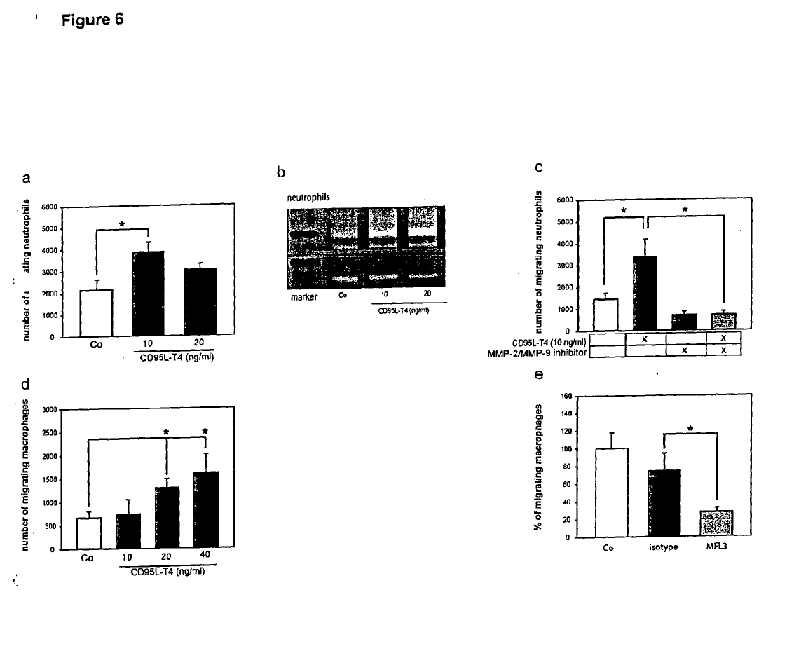

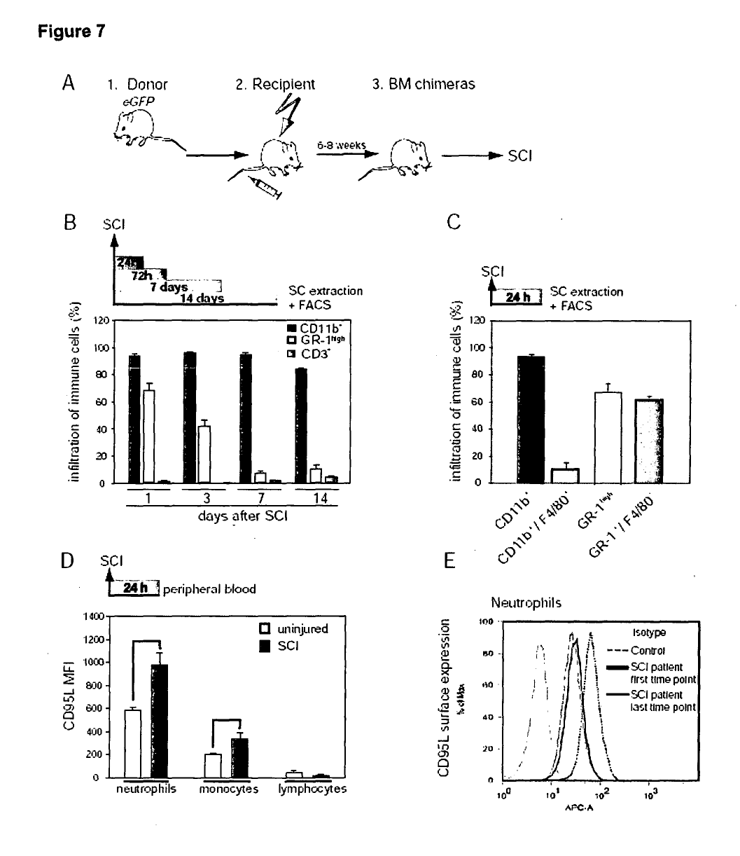

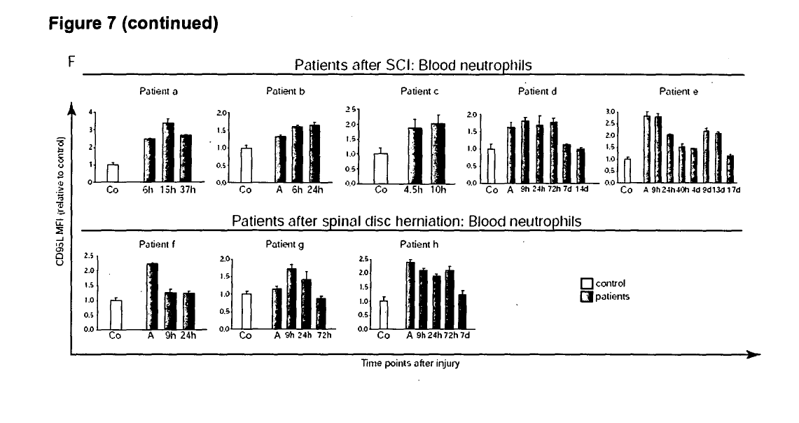

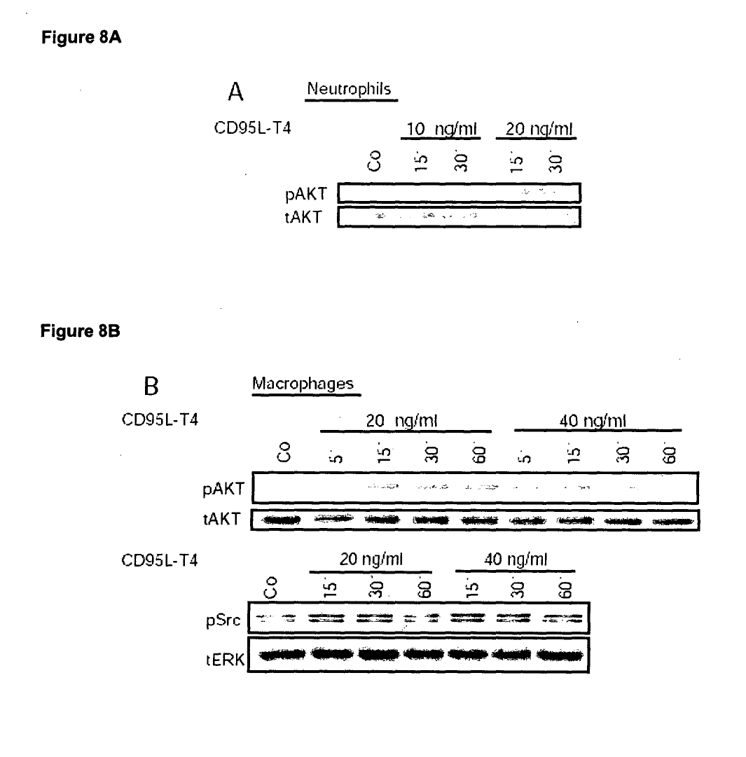

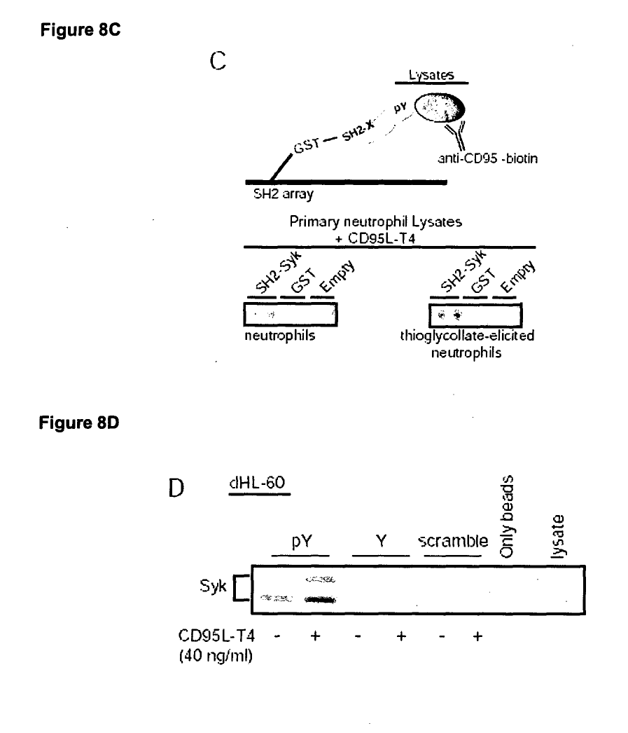

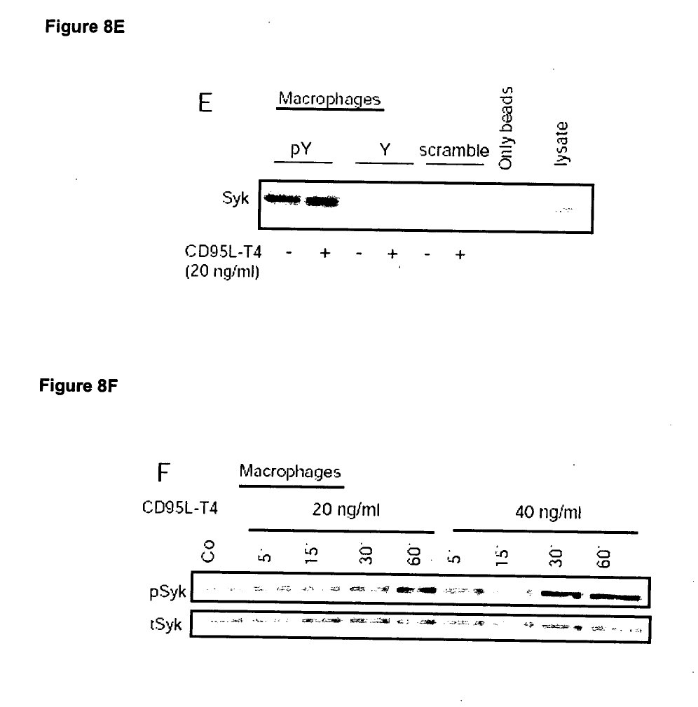

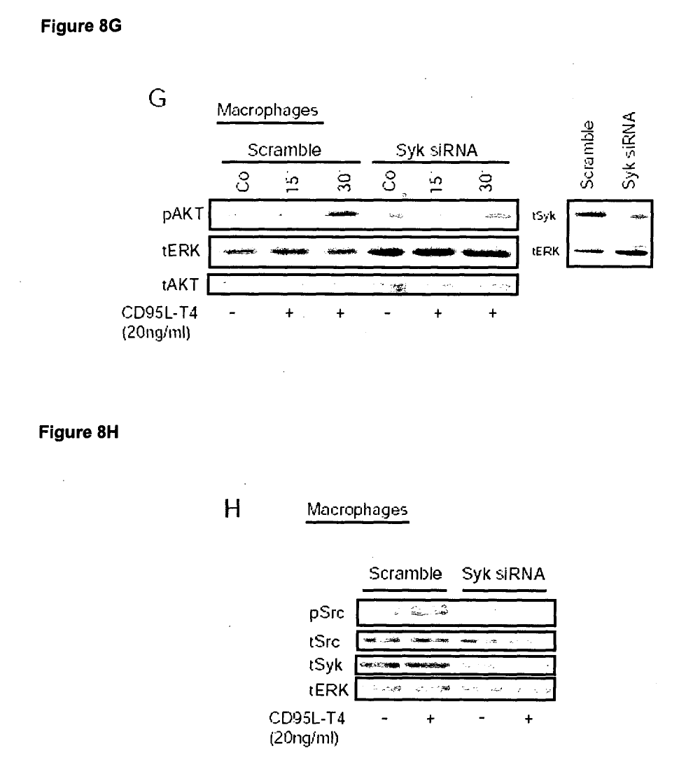

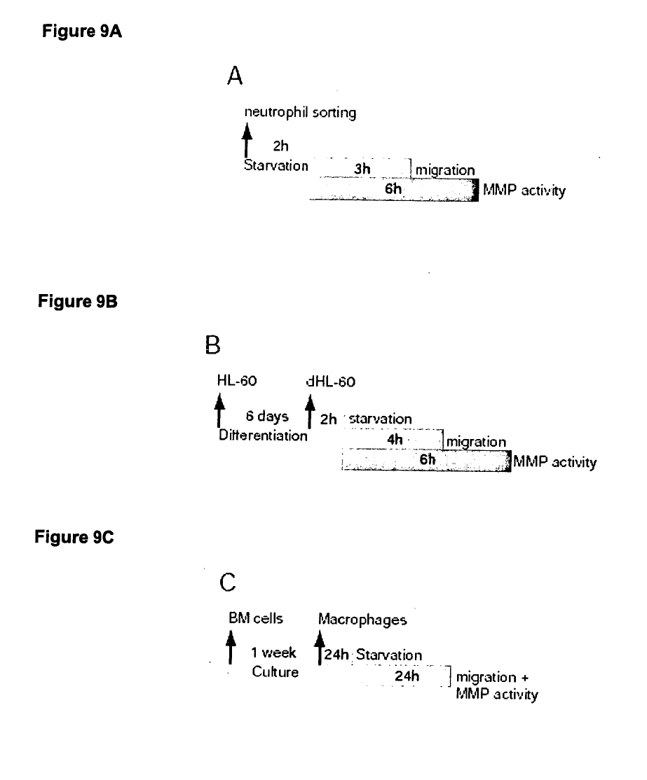

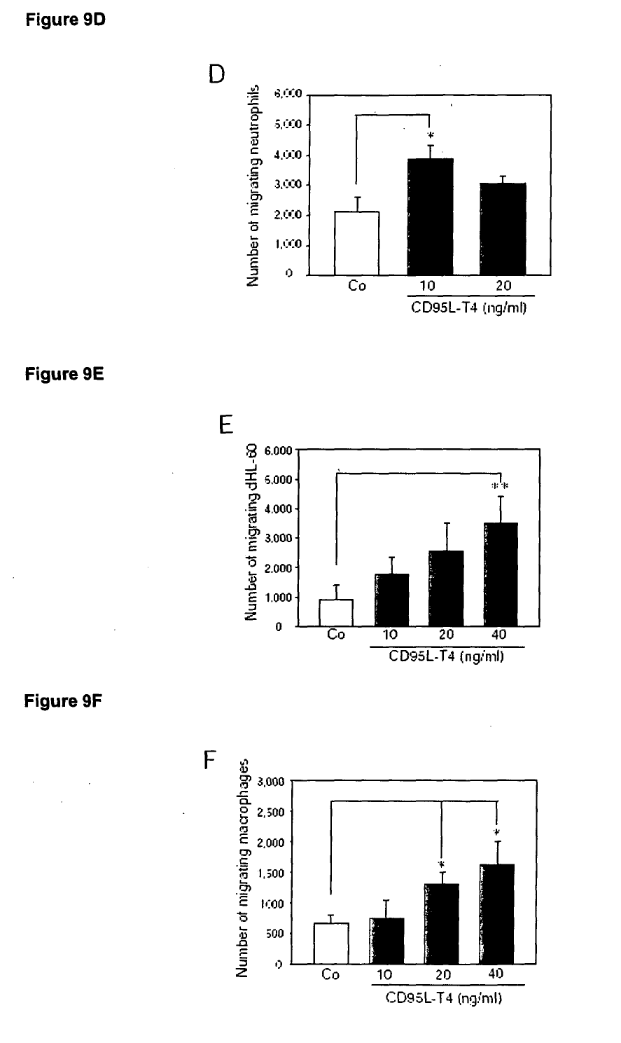

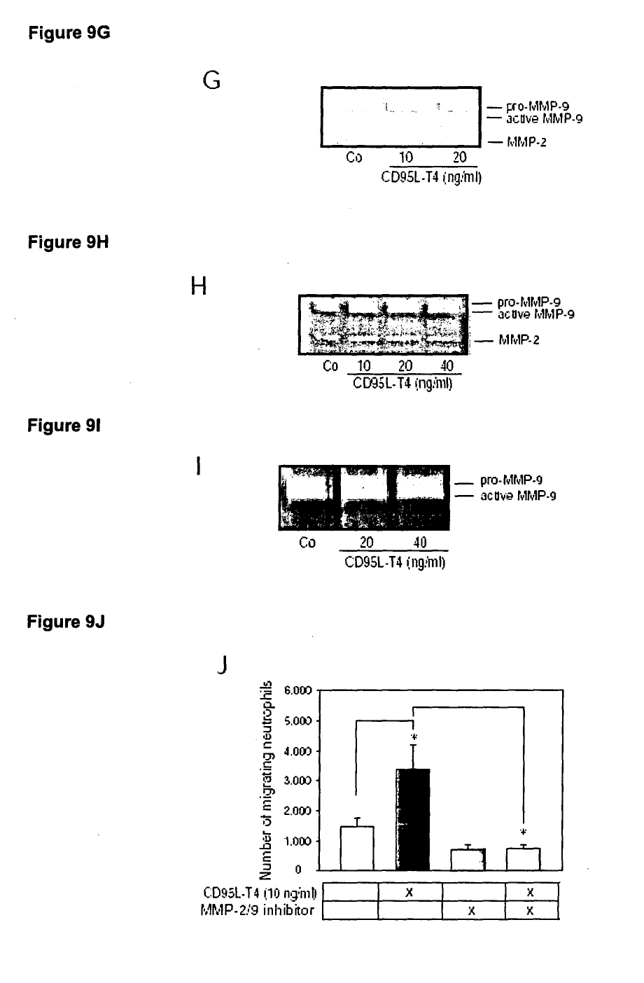

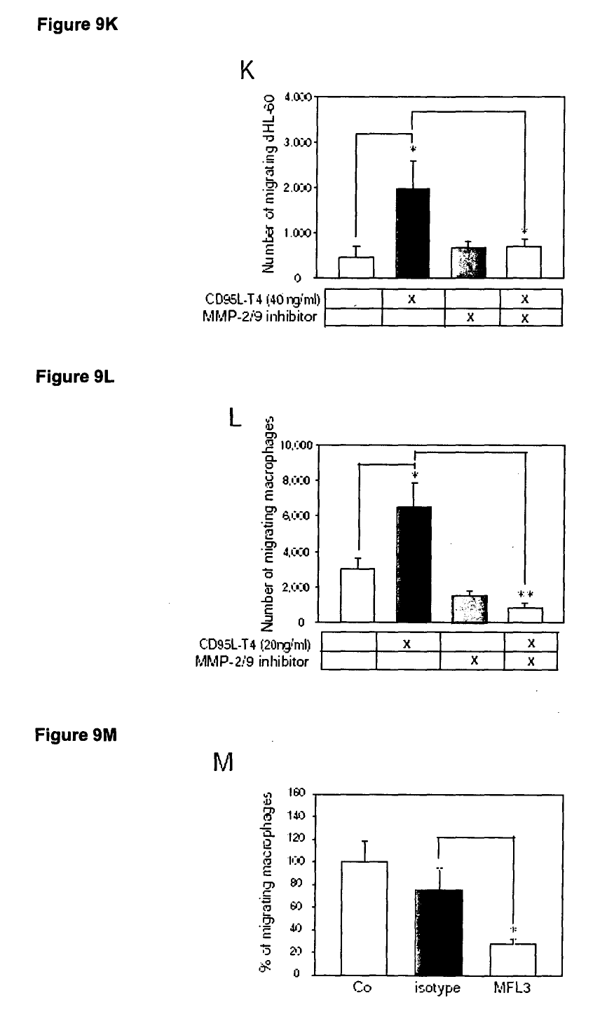

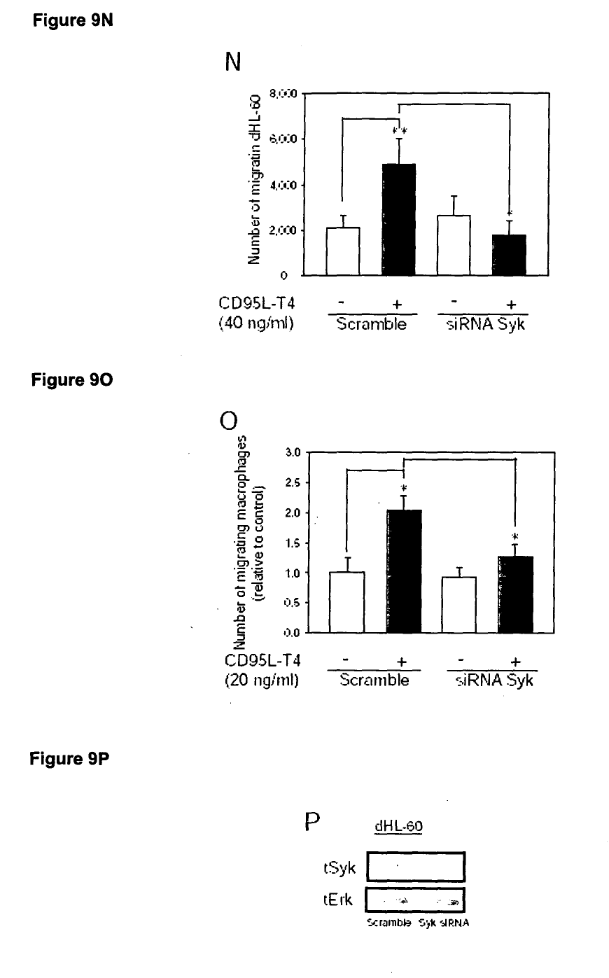

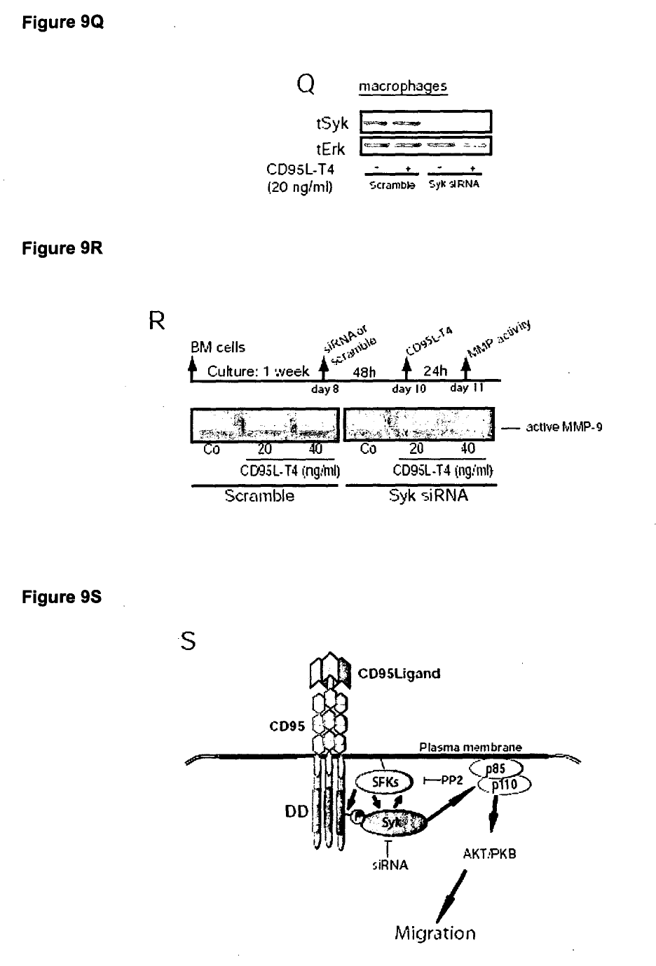

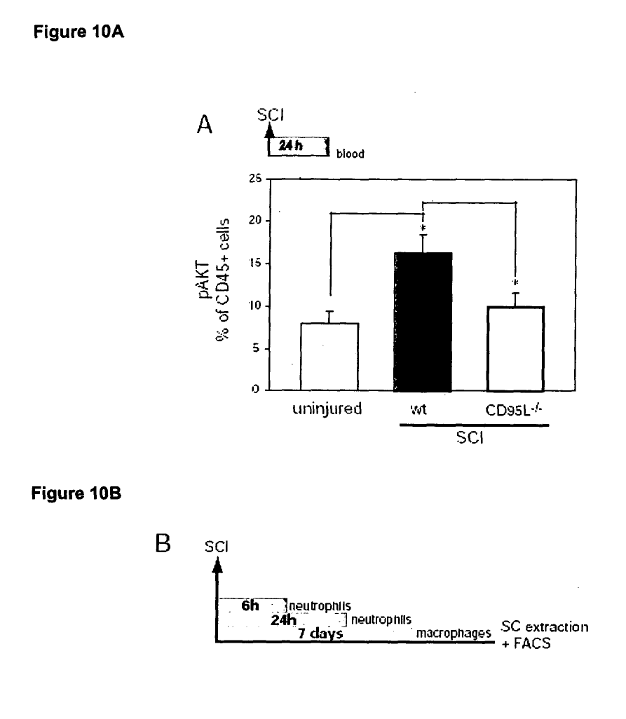

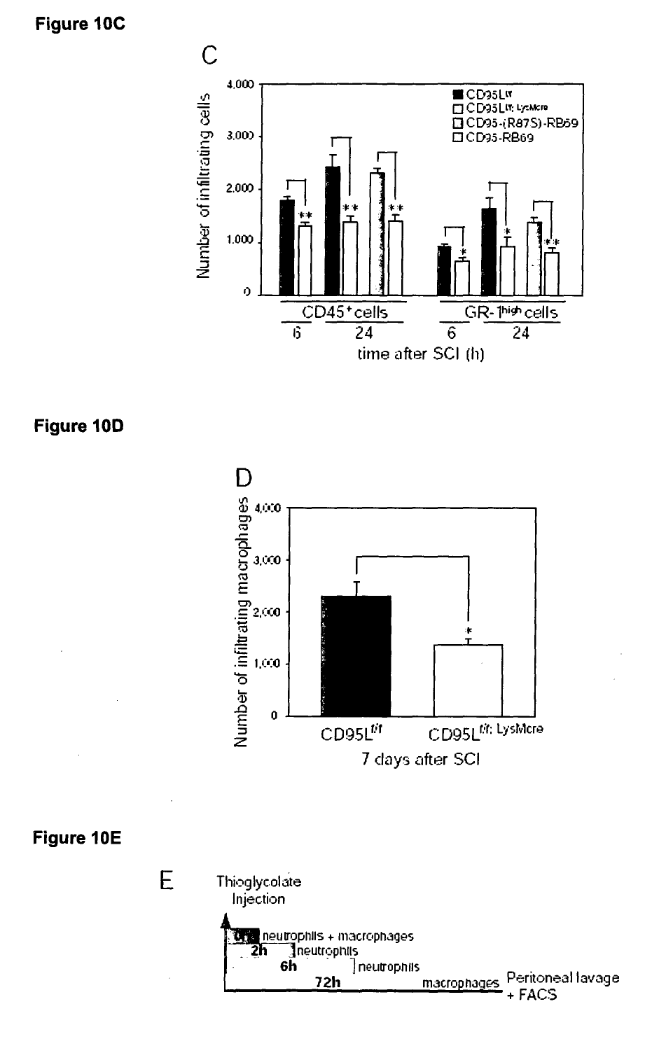

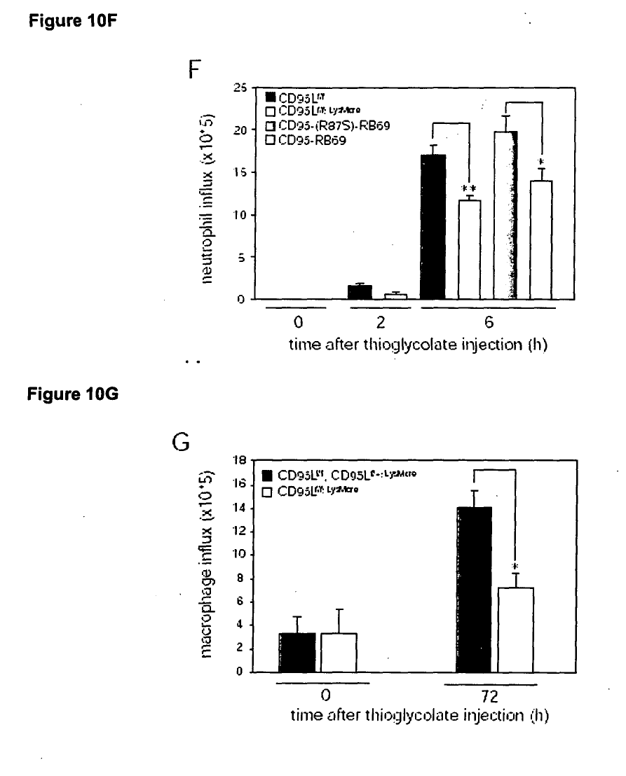

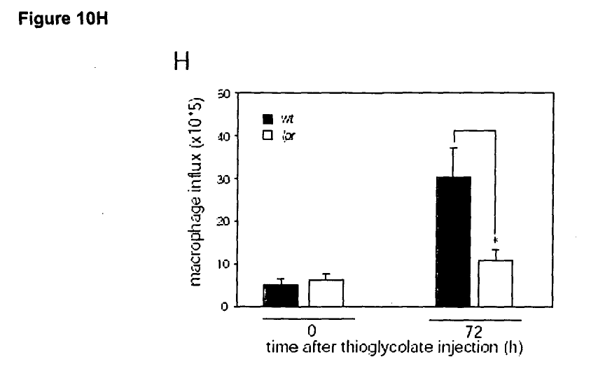

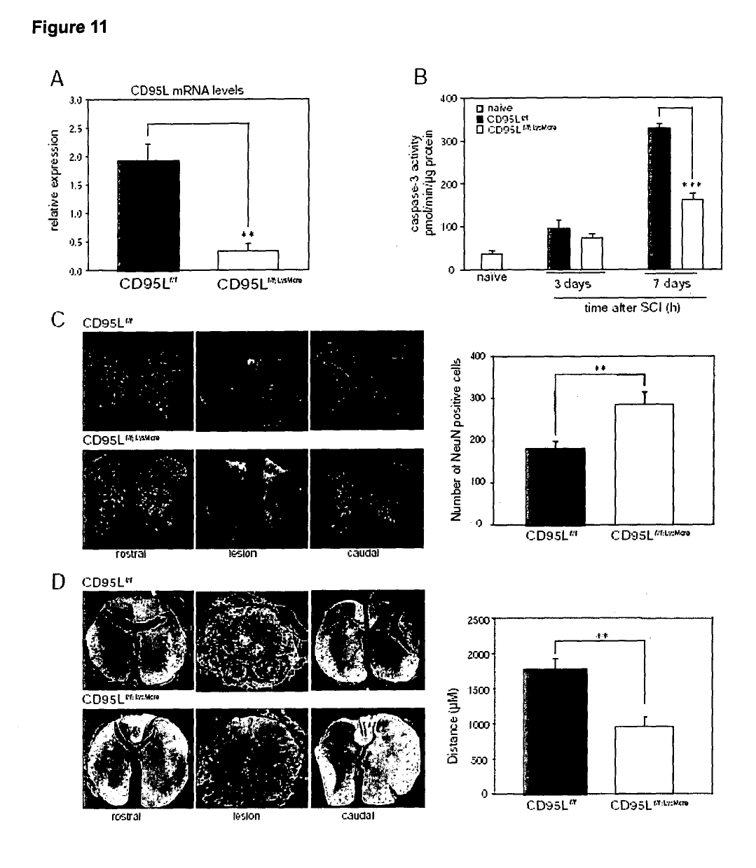

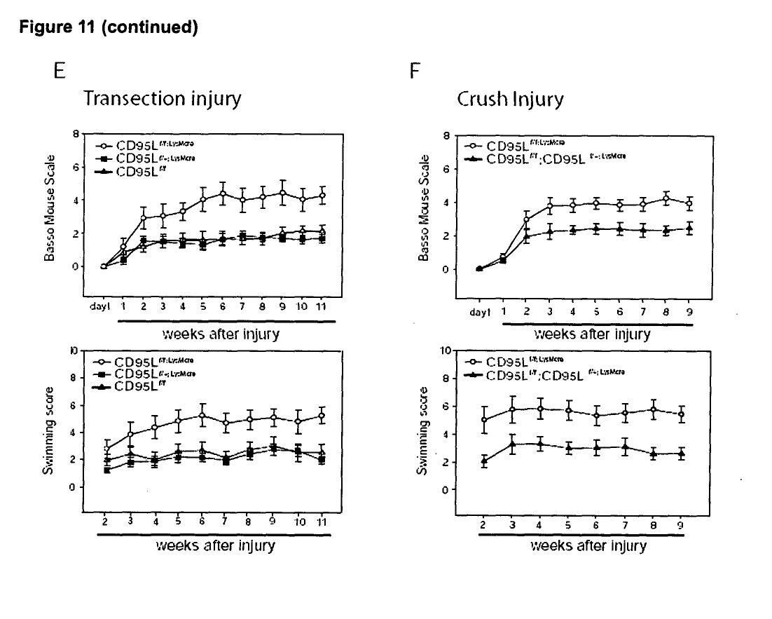

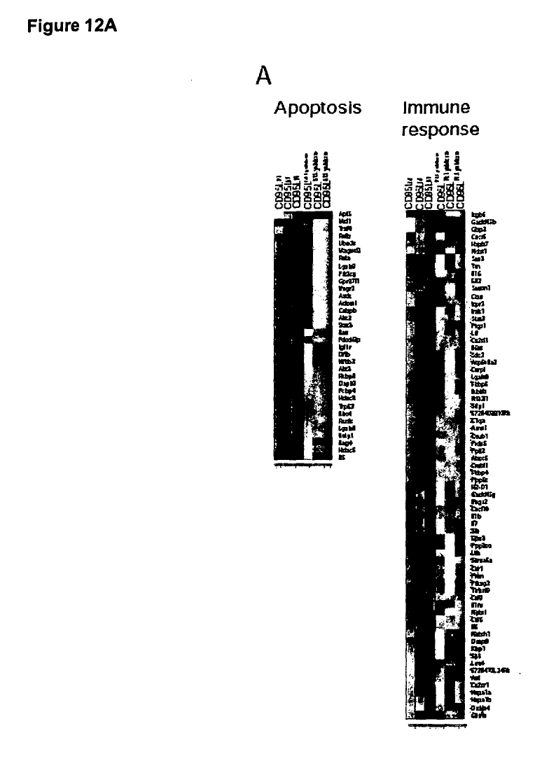

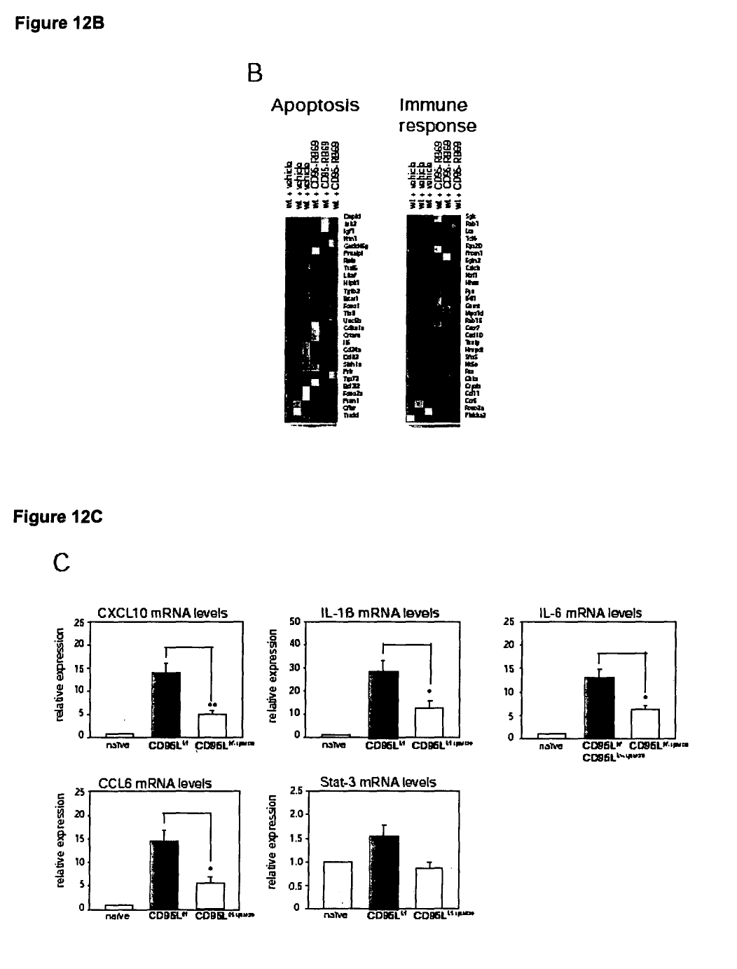

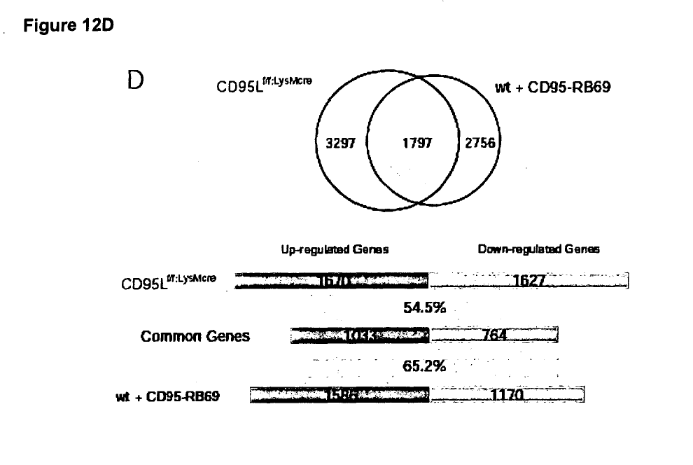

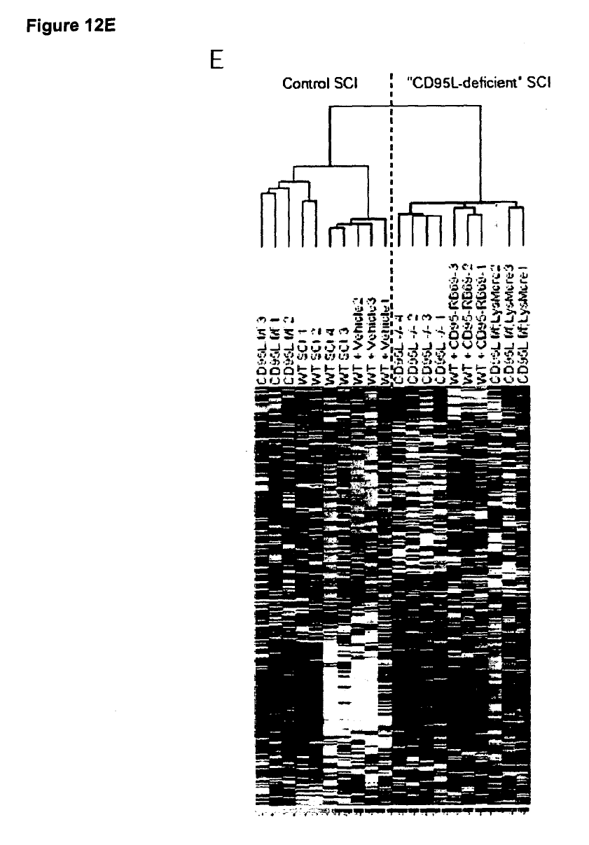

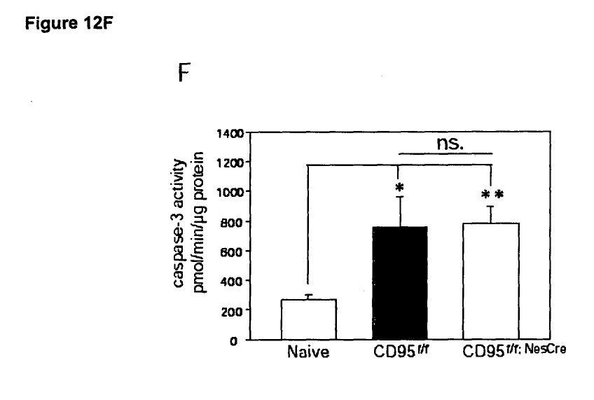

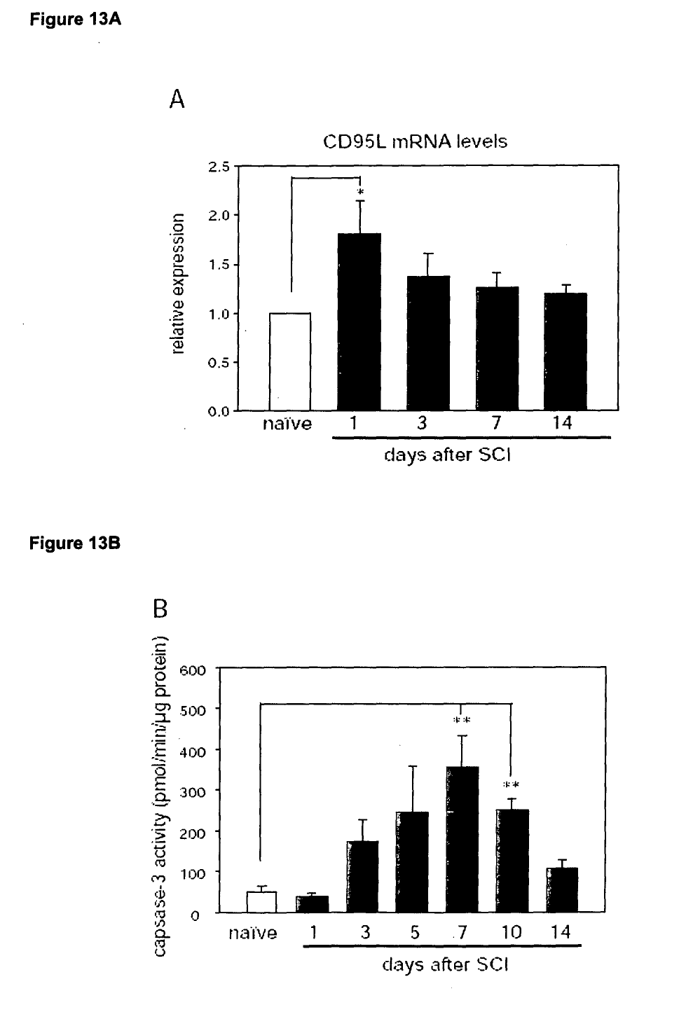

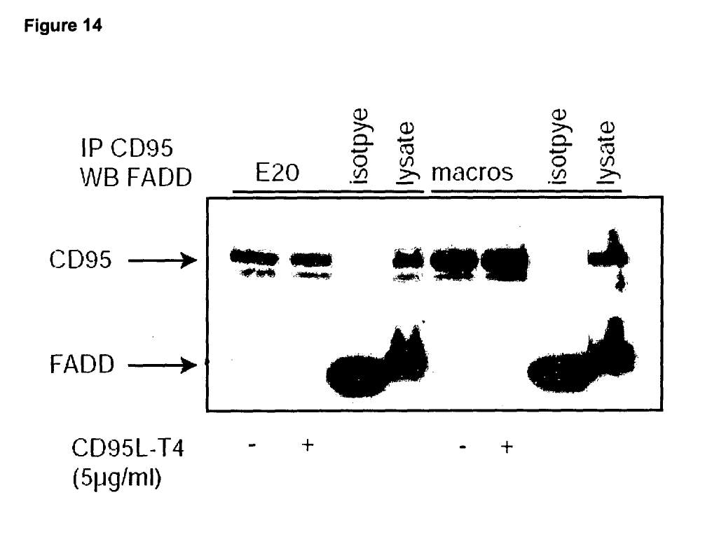

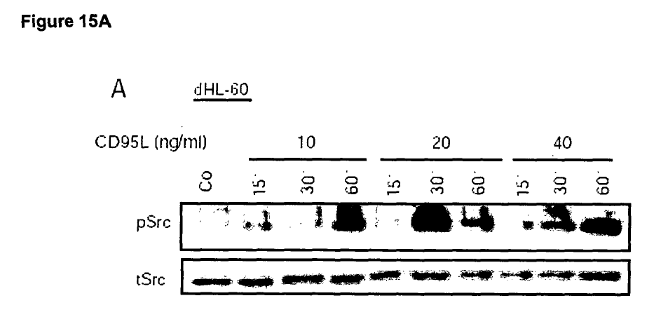

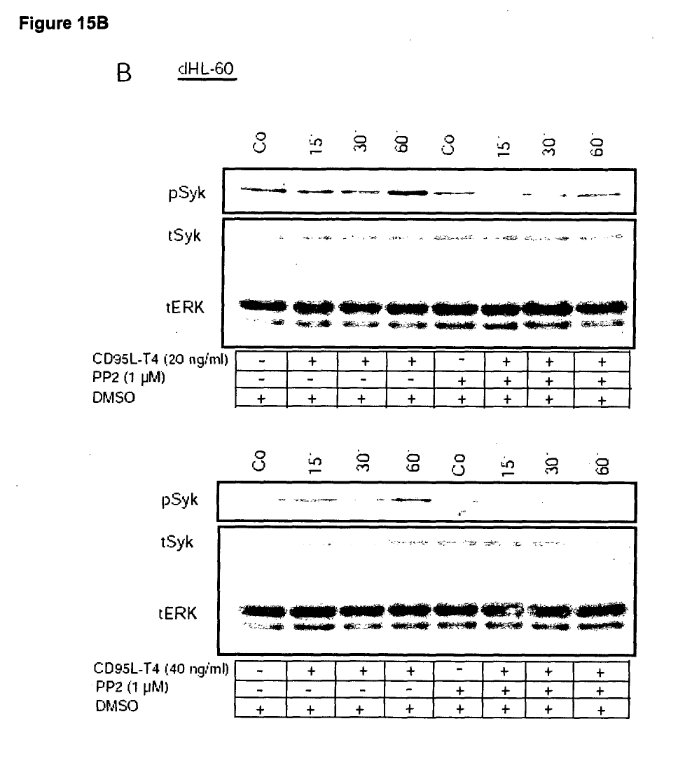

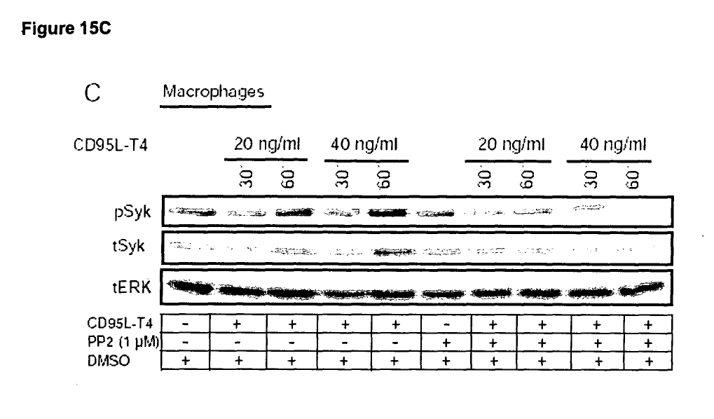

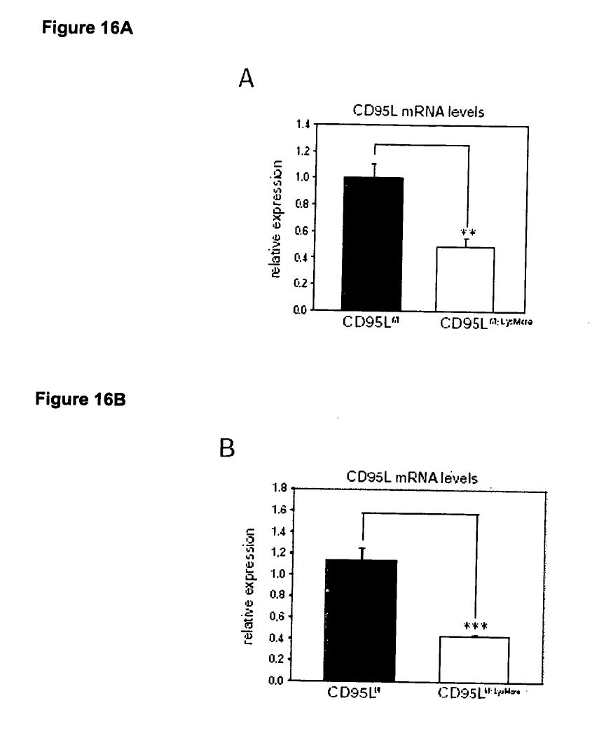

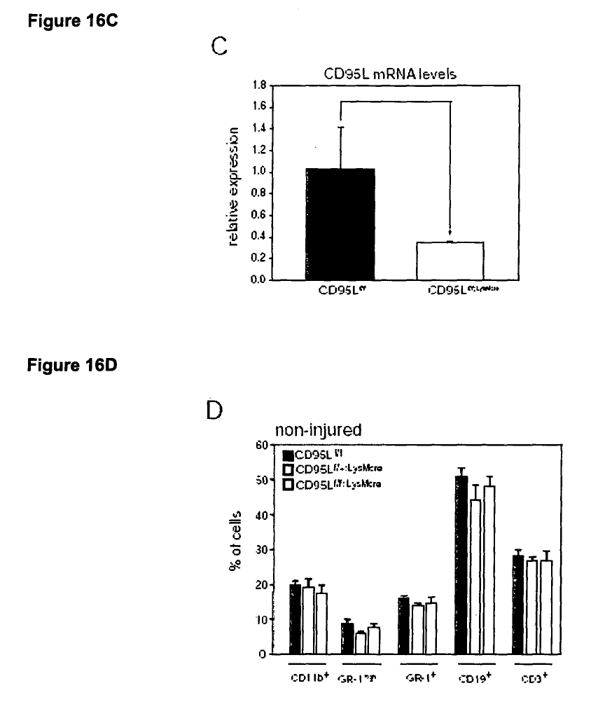

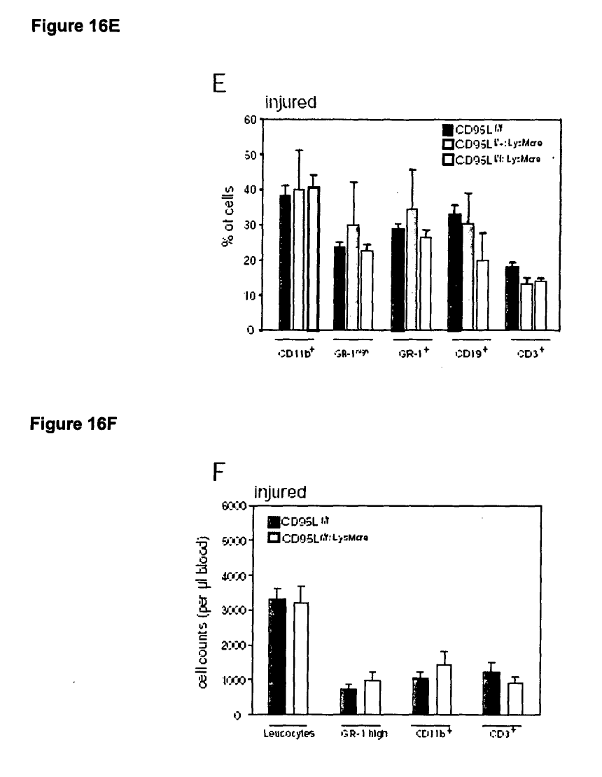

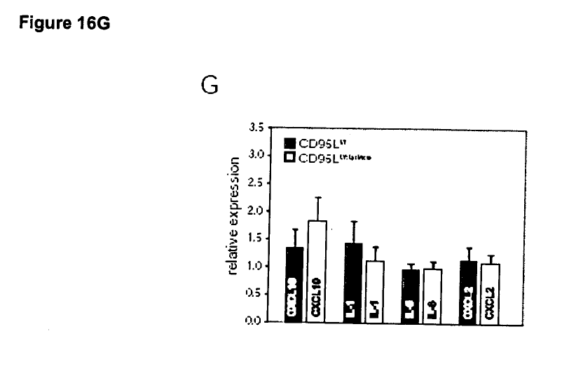

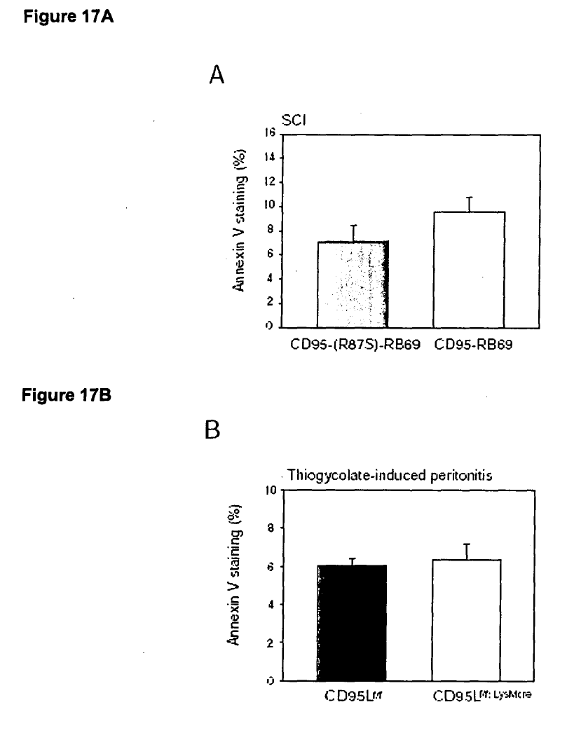

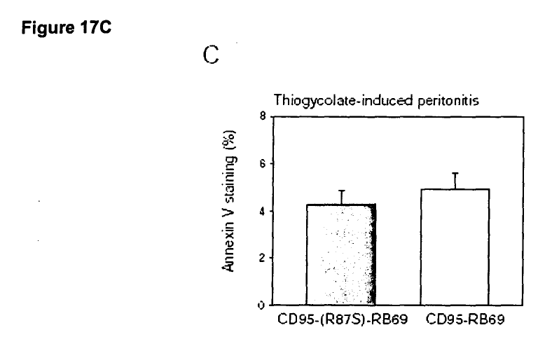

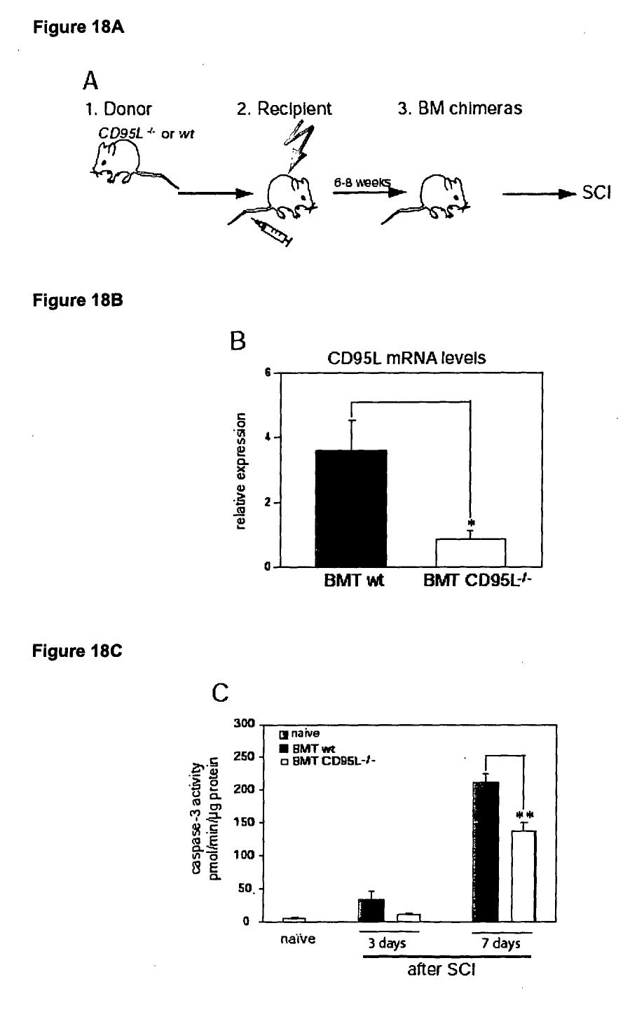

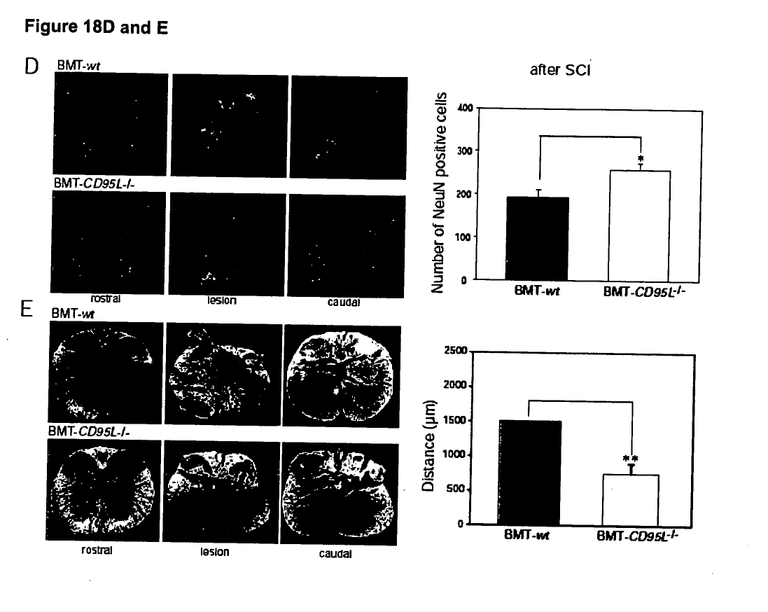

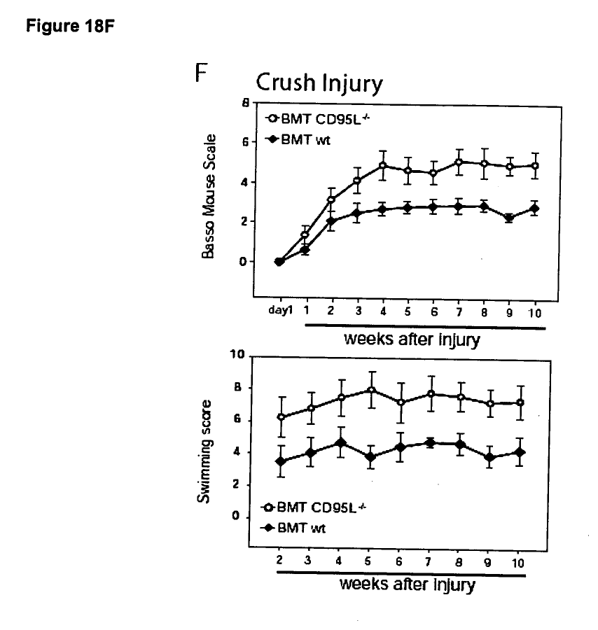

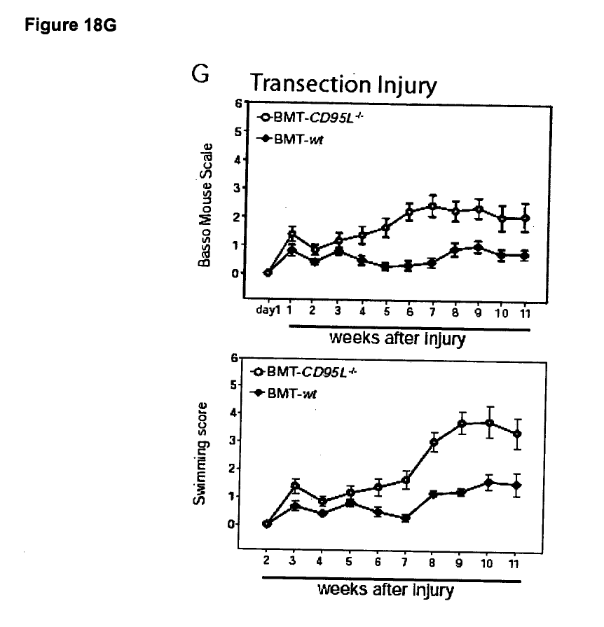

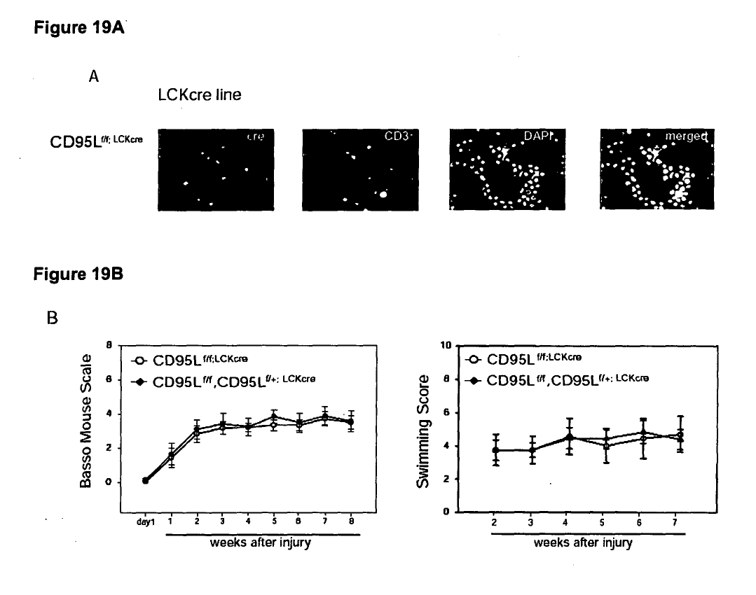

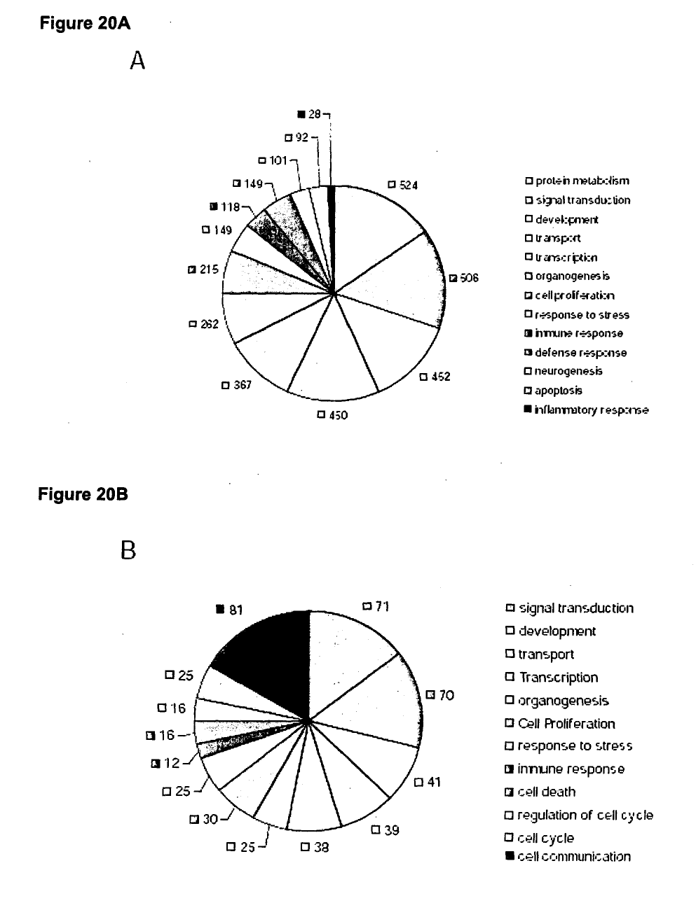

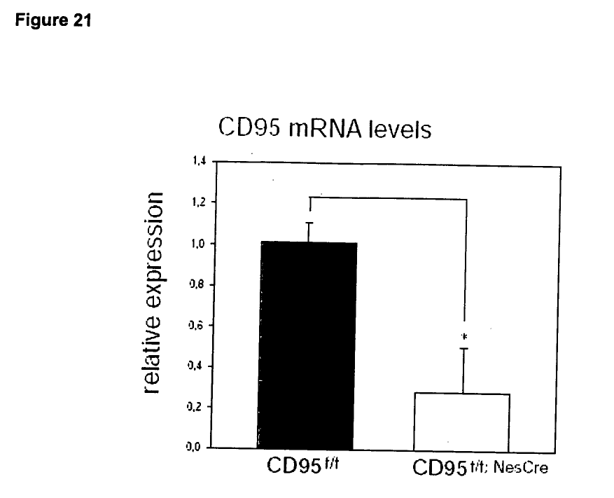

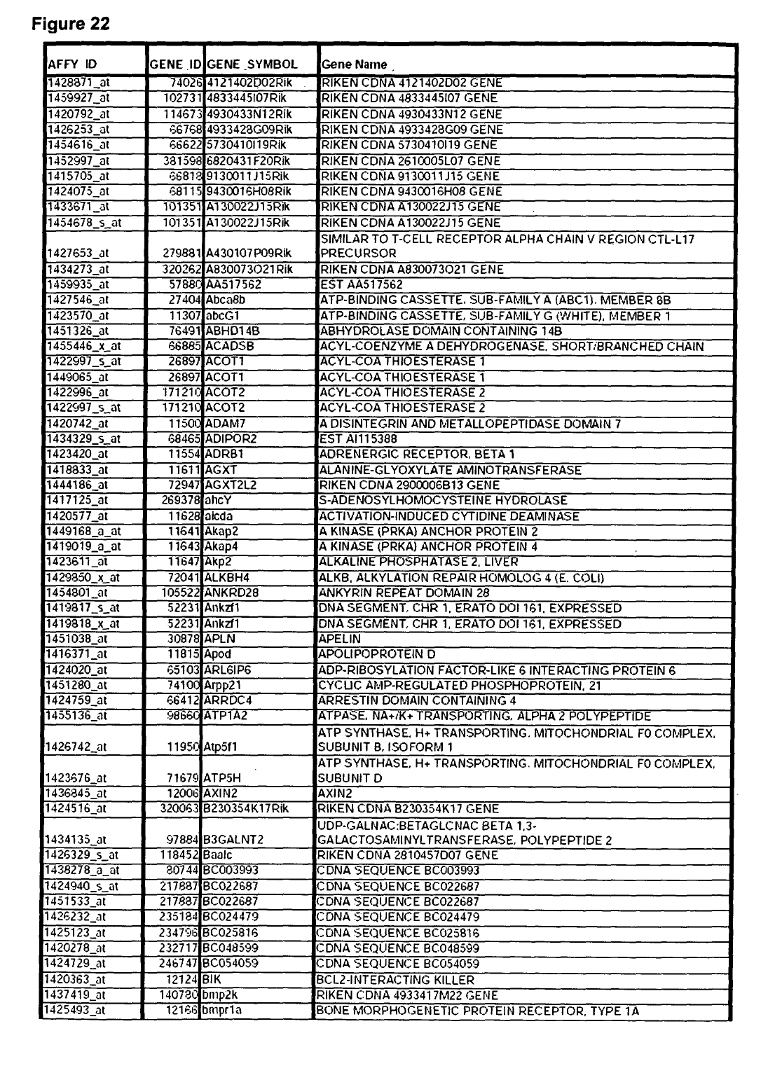

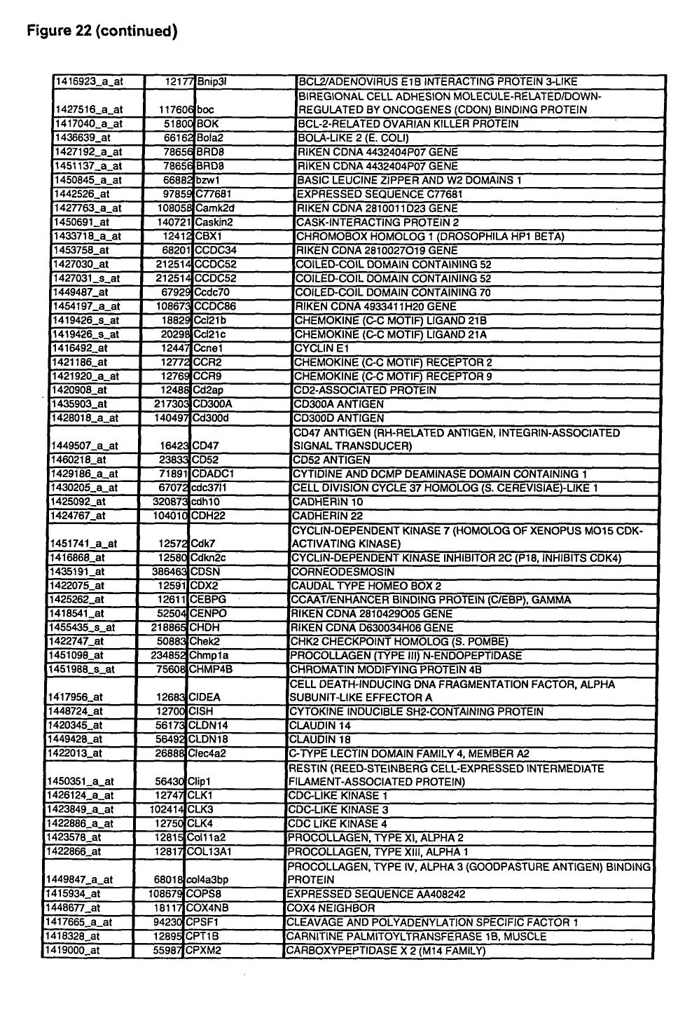

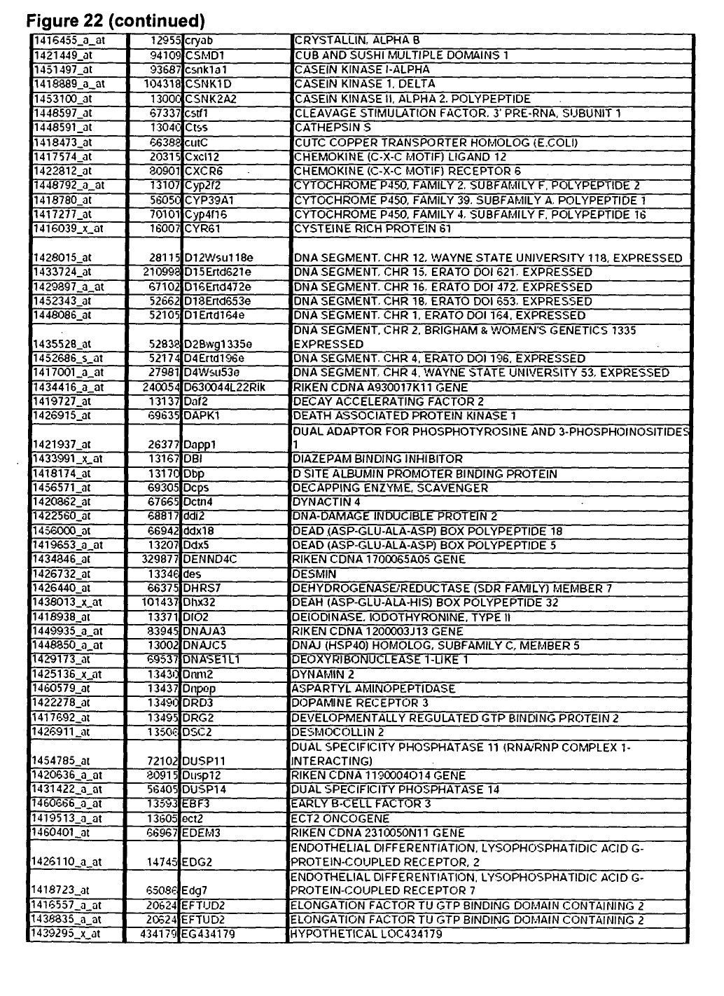

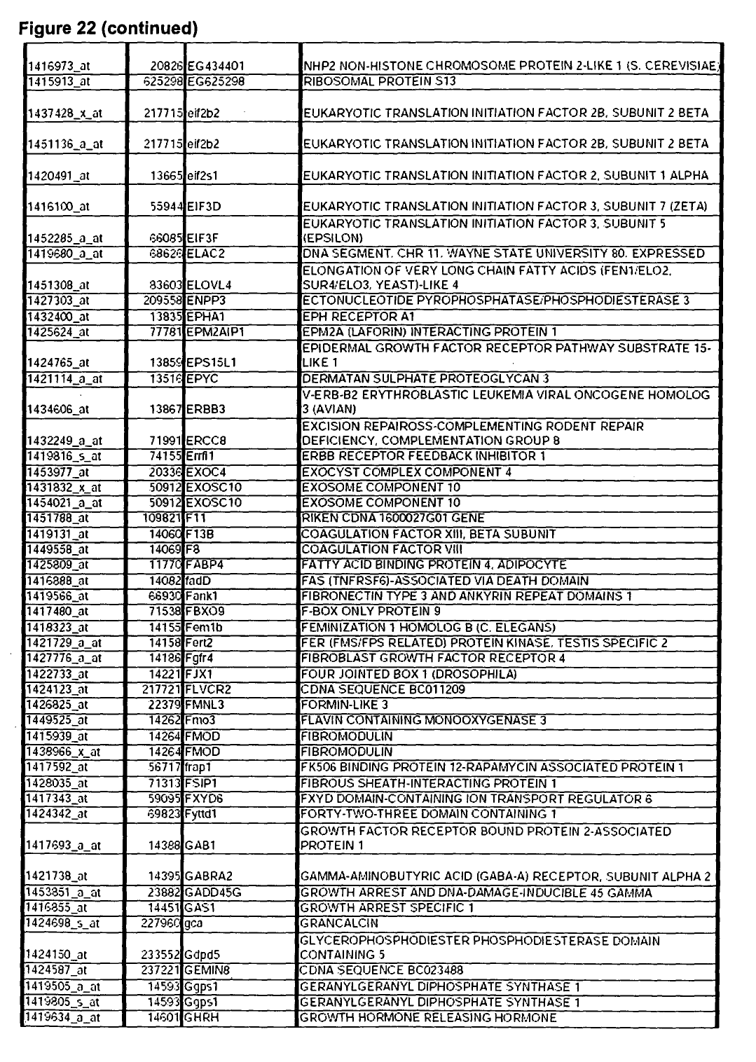

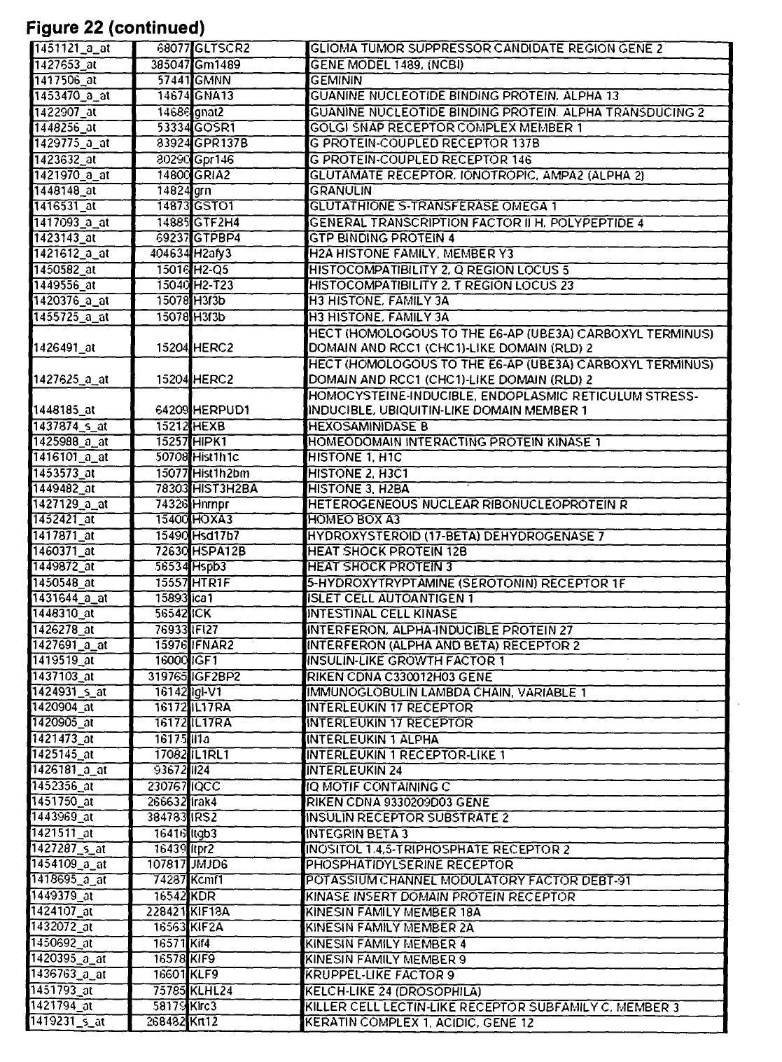

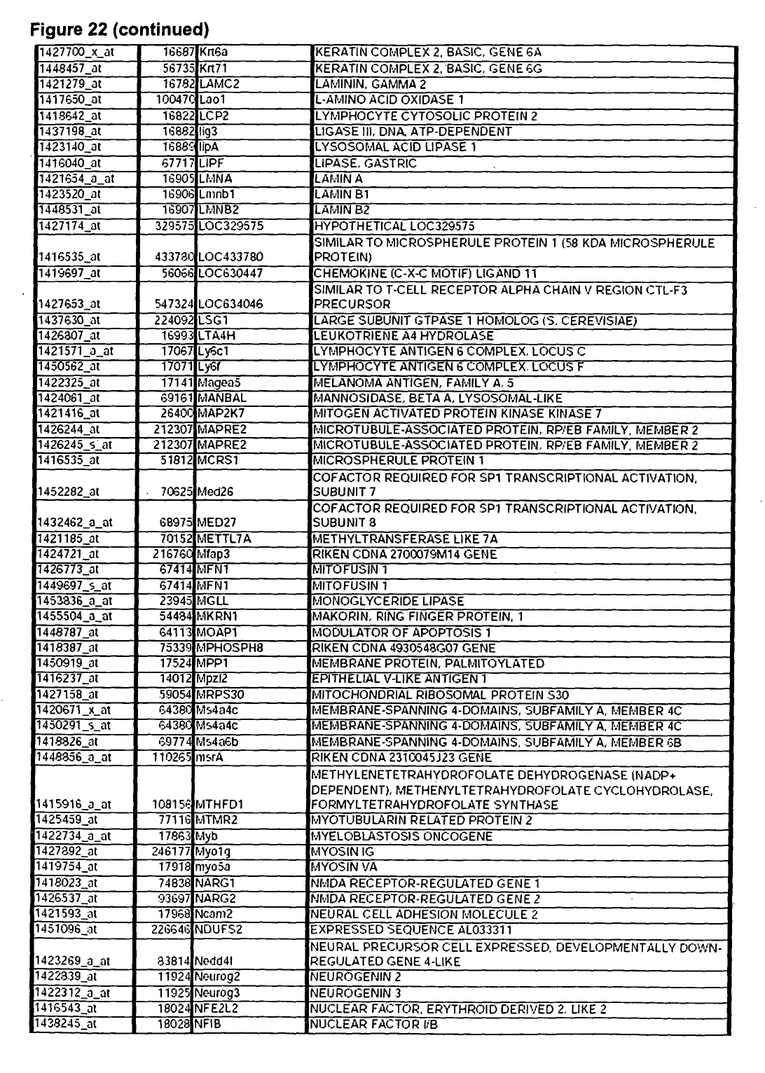

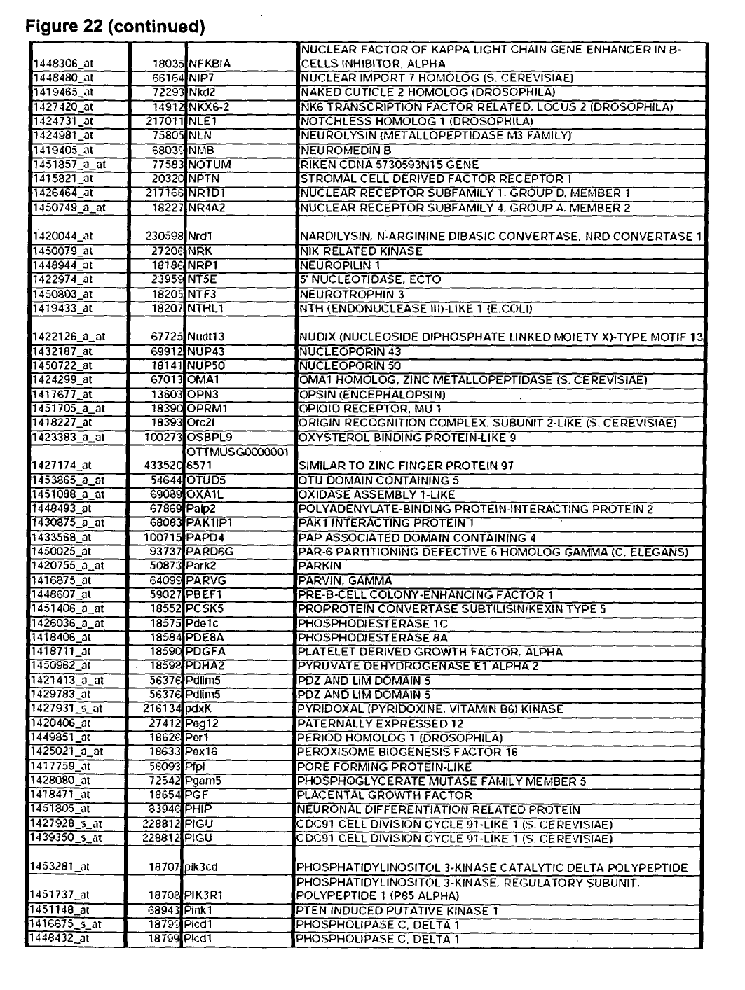

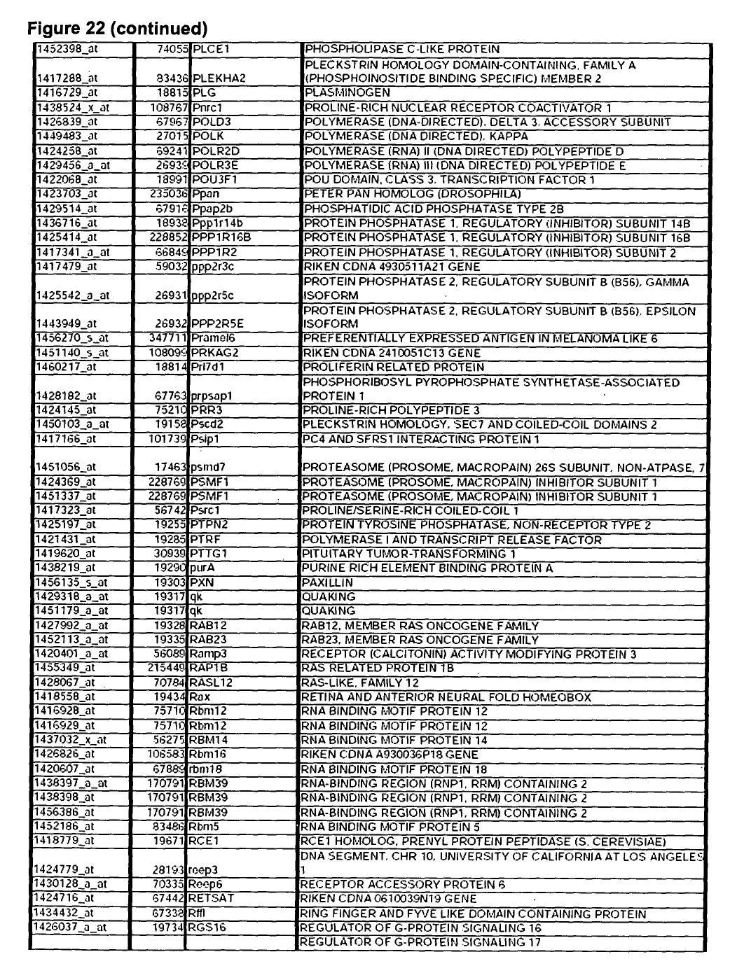

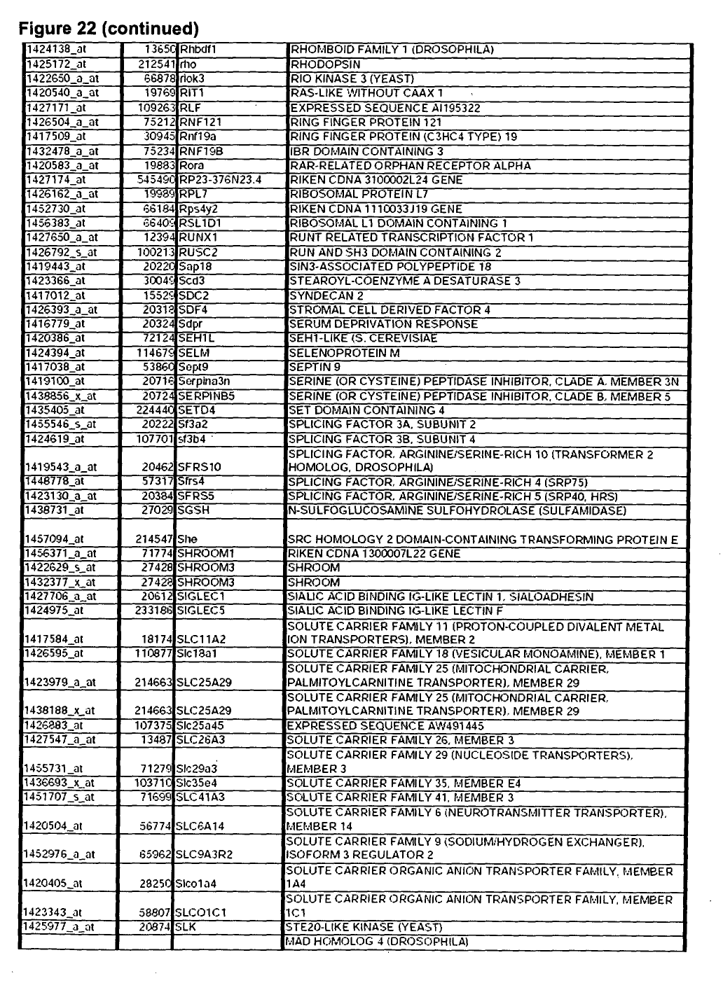

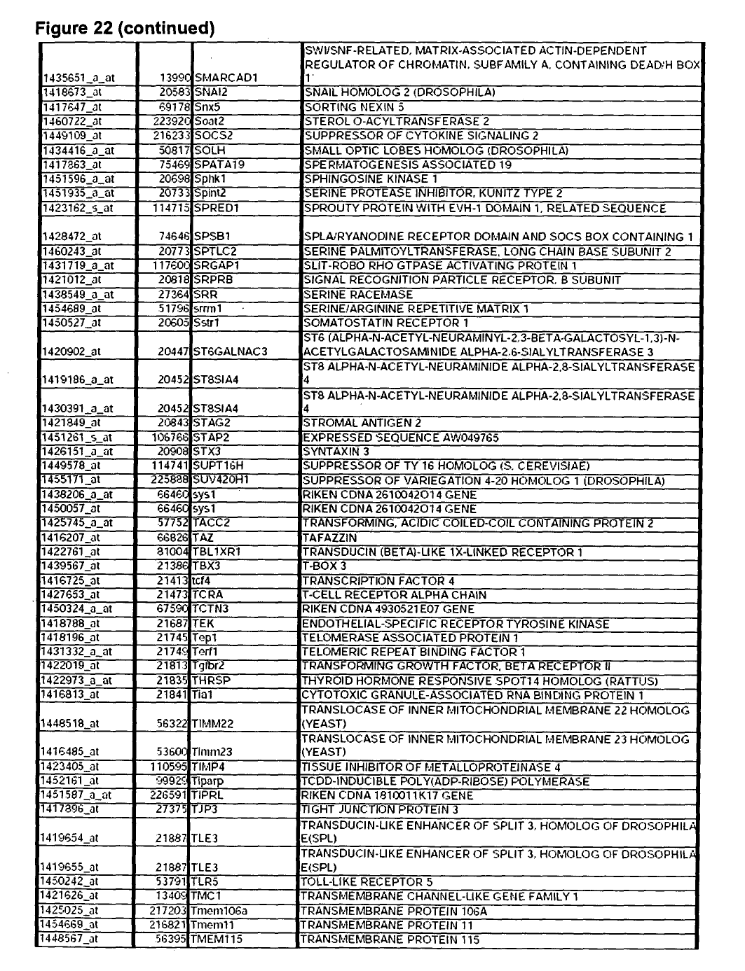

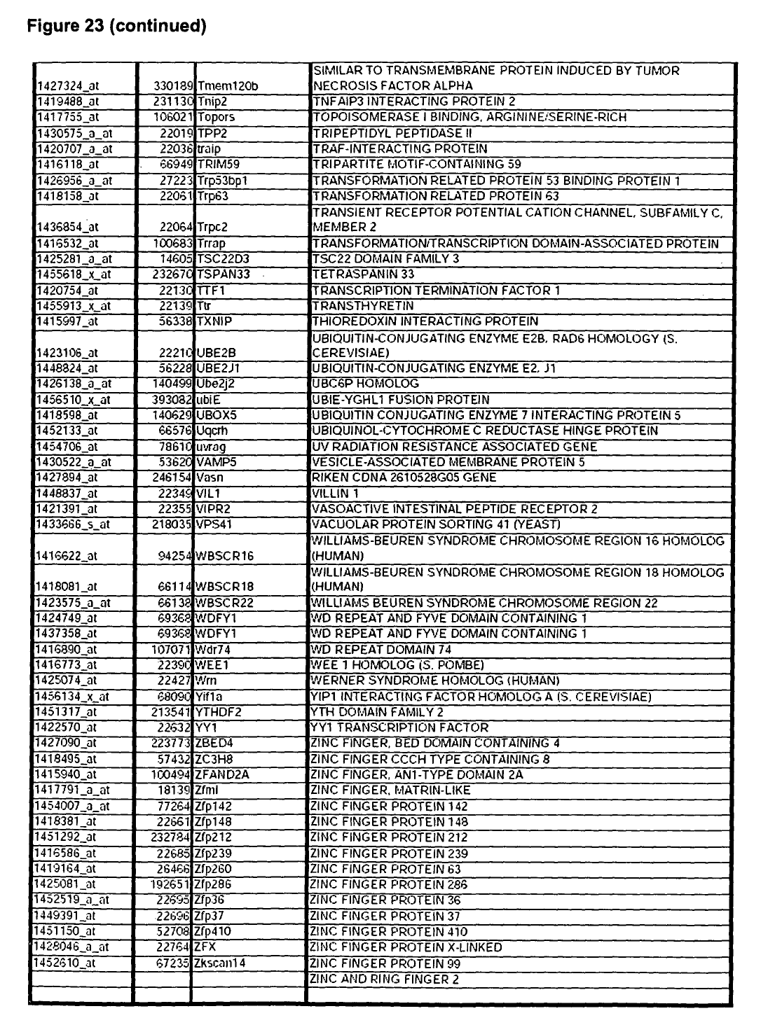

Macrophage recruitment to the site of the lesion can be driven by the previously recruited neutrophils. To uncouple the possible influence of neutrophils on macrophage infiltration, we separately studied CD95L-induced migration of neutrophils and macrophages in vitro in a two-chamber transmigration assay. Migration of bone marrow-derived neutrophils significantly increased upon treatment with CD95L (FIG. 6A). The increased migration was accompanied by increased activity of the matrix-metalloproteinase-9 (MMP-9) (FIG. 6B). Accordingly, pharmacological inhibition of MMP-9 and -2 abolished CD95L-induced migration of neutrophils (FIG. 6C). Furthermore, exogenous and endogenous CD95L increased macrophage migration in vitro (FIG. 6D). These findings demonstrate that CD95L directly acts on neutrophils and macrophages to increase their recruitment to the lesion site.

How does CD95L increase migration? In malignant glioma cells we have recently reported increased migration upon CD95L(5). In these cells the Src family kinase Yes and the p85 subunit of Phosphatidylinositol-3-Kinase (PI3K) get recruited to CD95 and activated upon CD95L. Thereafter the AKT/βcatenin pathway becomes activated leading to the final induction of MMP-9 expression. To address if PI3K is also needed in CD95-induced migration of myeloid cells, bone marrow derived neutrophils and macrophages were stimulated with CD95L and phosphorylation of the PI3K target AKT assessed. Phosphorylation and thus, activation of AKT was induced upon CD95L in both neutrophils and macrophages. As previously described for glioblastoma cells, AKT activation by CD95L in macrophages exhibited a dose-bell shape. We were unable to detect recruitment of FADD to neutrophils' or macrophages' CD95 upon treatment with CD95L, whereas treatment with CD95L of the thymoma cell line E020 efficiently recruited FADD to CD95. Further confirmation of the lack of FADD recruitment to CD95 and thus, of CD95-induced apoptosis is given by the missing differences in the rate of spontaneous apoptosis between neutrophils lacking CD95 activity and their respective controls after thioglycollate activation and SCI.

At present, the only treatment that shows a modest therapeutic benefit in spinal injured patients is the potent anti-inflammatory drug, methylprednisolone sodium succinate (MPSS). Patients treated with MPSS within the first 8 h of injury had significantly improved motor and sensory function compared to patients receiving placebo, naloxone, or MPSS at later time points (9). The required immediate use after injury indicates its major role in modulating the acute inflammatory response. Accordingly, depletion of circulating neutrophils, inhibition of neutrophil-related proteolytic enzyme activities or inhibition of neutrophils adhesion resulted in improved motor recovery of spinal cord injured mice (10). It is however noteworthy, that neutrophils also play an important role in cleaning the injury site and limiting bacterial infection. Thus therapies should aim at creating an inflammatory response devoid of devastating effects, such as CD95L-induced cell death of bystander cells (Brown and Savill, 1999), while still providing the beneficial effects. We therefore believe that a controlled modulation of the CD95L effects should provide a beneficial inflammatory response after SCI.

### 2. Behavioral Assessment of Anti-CD95L-Treated Mice

### 2.1 Materials and Methods

### 2.1.1 Animals

Animals used are described in the table below. CD95L^{-/-} were described previously (Karray et al., 2004) and C57BL/6J mice were purchased from Charles River Laboratories. CD95L floxed mice (Karray et al., 2004) were bred with LysM Cre mice (Jackson Laboratory) and LCK Cre mice (a kind gift from Gunter Hammerling) in order to deplete CD95L in myeloid cells or T cells, respectively. Mice that ubiquitously express an enhanced green fluorescent protein were a kind gift of Bernd Arnold. For experiments animals were age-matched and used at 12-14 weeks of age. All animal experiments were performed in accordance with institutional guidelines of the German Cancer Research Center and were approved by the Regierungspräsidium Karlsruhe, Germany.

| | | Respective | |
|---|---|---|---|
| Mice | Description | controls | Experiments |
| BMT CD95L^{-/-} | Deletion of CD95L in the | BMT wt | Locomotor activity |
| (CD95L^{-/-}→ wt) | immune cell compartment | (wt → wt) | |
| BMT eGFP | Every immune cell is | Naive animals | Time kinetics of immune |
| (eGFP → wt) | eGFP positive. | | cell infiltration in the injured |
| | | | spinal cord |
| LysMcre line | Genetic deletion of C95L | CD95L^{f/f} | Locomotor activity |
| CD95L^{f/f; LysMcre} | in myeloid cells | or CD95L^{+/f; LysMcre} | Gene expression profiling |
| | (neutrophils, macrophages | Control littermates | Infiltration analysis |
| | but not resident microglia) | | Meta analysis |
| LCKcre line | Genetic deletion of CD95L | CD95L^{f/f; LCKcre} | Locomotor activity |
| CD95L^{f/f; LCKcre} | in T cells | or CD95L^{+/f; LCKcre} | |
| | | Control littermates | |
| wt mice treated with | Pharmacological block of | CD95-(R87S)-RB69- | Infiltration analysis |
| CD95-R869 | CD95L with a CD95 trimer | or vehicle-treated wt | Meta analysis |
| | | mice | |
| CD95L^{-/-} | Genetic Ubiquitous | wt | Meta analysis |
| | deletion of CD95L | | |

### 2.1.2 Spinal Cord Injury

SCI models: Transection injury of the spinal cord was performed as previously described (Demjen et al., 2004). For the crush injury model, forceps were held on the spinal cord for 15 seconds resulting in a lateral compression of the spinal cord (Plemel et al., 2008). Immediately following injury and for an additional week mice received antibiotics (Gentamycin, 5 ml/kg of a 0.2 mg/ml solution) to prevent infections. Post-operative care included housing of the animals at 27° C., food and water ad libitum, and manual expression of the bladders once daily.

### 2.1.3 Staining of Human Blood Samples

All experiments on human blood were performed in accordance with institutional guidelines of the German Cancer Research Center and were approved by the Ethic Commission in Mainz. Once the blood of a patient and a respective healthy control was collected, erythrocyte lysis was performed followed by fixation with 4% PFA. All the time points belonging to one patient as well as 5-6 respective control samples were stained together. For this, NOK-2 (BD, Pharmingen) or the respective IgG2% isotype (Acris) were incubated 1 hour on ice followed by 30 minutes incubation with the secondary antibody (anti-mouse APC, BD). Thereafter, samples were analyzed for CD95L expression on the surface of human neutrophils and lymphocytes by flow cytometry. Neutrophils were either identified by CD66b positive cells or by their FSC/SSC.

### 2.1.4 Anti-CD95L Treatment

Mice were treated intravenously 5 minutes after SCI or induction of thioglycolate-induced peritonitis with 50 μg (solved in 200 μl sterile PBS) of either CD95-RB69 or a mutated form, CD95-(R87S)-RB69, which is unable to bind CD95L.

### 2.1.5 Behavioral Assessment

All behavioral tests were performed by two independent observers in a double-blind manner weekly for 9-11 weeks after injury. The general locomotor performance of the animals was assessed using the Basso Mouse locomotor rating Scale (BMS) and the swimming test, assessed as previously described (Demjen et al., 2004). For the BMS, animals were additionally tested at the first day after injury. Any mouse showing a BMS score over 0.5 at day 1 was excluded from further studies.

### 2.1.6 Statistical Evaluation

All statistical summary data including the sample size and results of statistical evaluations are listed in the table below. For behavioral experiments, the overall improvement in mice compared to the control group was statistically analyzed by using the Koziol test (Koziol et al., 1981), a non-parametric test appropriate for longitudinal data which allows to analyze these data combined over time. Statistical analyzes of all other endpoints was performed by using the standard unpaired Student t-test. No formal test for normality was applied in view of the small sample sizes when Student's t test was applied. All data were presented as mean±standard error of the mean (SEM). Statistical significance was reported by the p-value of the statistical test procedures and assessed, significant *p<0.05; strongly **p<0.01 and highly ***p<0.001 significant. All statistical analyses were performed with the program package ADAM of the Biostatistics Unit of the German Cancer Research Center, DKFZ.

| | | | | | | Number of |
|---|---|---|---|---|---|---|
| | Figure + | | | | | mice or of |
| | Supple- | | | | Statis- | experiments |
| | mentary | | | p-Value | tical | For the in |
| Validation of | Figure (S) | Studied mice | Control mice | related to the controls | Test | vitro studies |
| CD95L MFI | 1 | Wt + SCI | Wt | Neutrophils: p = 0.02 | t-test | n = 4/group |
| | | Neutrophils: 975.67 ± 102.83 | Neutrophils: 585.34 ± 21.94 | Macrophages: p = 0.04 | | |
| | | Monocytes: 339.17 ± 47.40 | Monocytes: 197.27 ± 10.49 | T-cells: not sig. | | |
| | | T-cells: 18 ± 4.48 | T-cells: 46.33 ± 12.10 | | | |
| In vitro | 3 | CD95L-T4 treated | Untreated (control) | Related to control | | |
| migration | | | | | | |
| assays | | | | | | |
| Neutrophil | 3d | cell count ± SEM | cell count ± SEM | (10 ngml): p = 0.04 | t-test | 4 |
| | | (10 ngml): 3862.69 ± 459.19 | Control: 2134.06 ± 493.18 | (20 ngml): Not sig. | | independant |
| | | (20 ngml): 3041.92 ± 263.95 | | | | experiments |
| dHL-60 | 3e | cell count ± SEM | cell count ± SEM | (10 ngml): Not sig. | t-test | 3 |
| | | (10 ngml): 1756.47 ± 485.01 | Control: 896.66 ± 485.01 | (20 ngml): not sig. | | independant |
| | | (20 ngml): 2560.82 ± 948.44 | | (40 ngml): p: 0.019 | | experiments |
| | | (40 ngml): 3485.45 ± 923.44 | | | | |
| Macrophage | 3f | cell count ± SEM | cell count ± SEM | (10 ngml): Not sig. | t-test | 4 |
| | | (10 ngml): 736.94 ± 316.06 | Control: 668.15 ± 138.08 | (20 ngml): p = 0.03 | | independent |
| | | (20 ngml): 1293.00 ± 201.04 | | (40 ngml): p = 0.045 | | experiments |
| | | (40 ngml): 1604.35 ± 403.79 | | | | |
| Migration | 3j-l | MMP-2 and -9 inhibition | Untreated (control) and | | | |
| after MMP-2/9 | | | CD95L-T4 treated | | | |
| blockade | | | | | | |
| neutrophils | 3j | cell count ± SEM | cell count ± SEM | Control to treated: p = 0.045 | t-test | 2 |
| | | Control: 713.00 ± 161.89 | Control: 1455.75 ± 280.42 | Control to control vs. MMP- | t-test | independent |
| | | 10 ng/ml: 724.69 ± 150.01 | 10 ng/ml: 3390.27 ± 805.85 | 2/9 inhibition: p = 0.05 | t-test | experiments |
| | | | | Treated to treated vs. | | |
| | | | | MMP-2/9 inhibition: | | |
| | | | | p = 0.023 | | |
| dHL-60 | 3k | cell count ± SEM | cell count ± SEM | Control to treated: p = 0.039 | t-test | 1 |
| | | Control: 658.09 ± 137.65 | Control: 454.76 ± 230.80 | Treated to treated vs. | t-test | experiment |
| | | 40 ng/ml: 683.09 ± 170.89 | 40 ng/ml: 1969.54 ± 610.93 | MMP-2/9 inhibition: | | |
| | | | | p = 0.05 | | |
| macrophages | 3l | cell count ± SEM | cell count ± SEM | Control to treated: p = 0.039 | t-test | 2 |
| | | 20 ng/ml treated with | 20 ng/ml treated: | Control to control vs. MMP- | | independent |
| | | MMP2/9 inhibition: | 4857.80 ± 1094.94 | 2/9 inhibition: p = 0.045 | | experiments |
| | | 323.80 ± 100.20 | | Treated to treated vs. | | |
| | | | | MMP-2/9 inhibition: | | |
| | | | | P = 0.001 | | |
| Basal | 3m | MFL3-treated | Untreated or isotpye | | | |
| migration of | | | treated | | | |
| macrophages | | | | | | |
| | 3j | % of migrating cells ± SEM | % of migrating cells ± SEM | To control: p = 0.002 | t-test | 2 |
| | | MFL3: 27.65 ± 5.13 | Control: 100.00 ± 18.39 | To isotype: p = 0.037 | t-test | independent |
| | | | Isotype: 74.58 ± 20.25 | | | experiments |
| | | | | | | which were |
| | | | | | | pooled |
| CD95L- | 3n, o | Syk Knockdown | Scramble | | | |
| induced | | | | | | |
| migration via | | | | | | |
| Syk | | | | | | |
| dHL-60 | 3n | cell count ± SEM | cell count ± SEM | | | 2 |
| | | Syk KD: 2618.29 ± 859.58 | scramble: 2078.04 ± 530.32 | scramble to scramble | t-test | independent |
| | | | | treated p = 0.0296 | | experiments |
| | | Syk KD treated: | scramble treated: | scramble treated to syk | | |
| | | 1797.30 ± 636.03 | 4893.35 ± 1099.50 | knockdown treated | | |
| | | | | p = 0.026 | | |
| Macrophages | 3o | cell count ± SEM | cell count ± SEM | scramble to scramble | t-test | 3 |
| | | Syk KD: 0.94 ± 0.15 | scramble: 1.0 ± 0.25 | treated p = 0.0059 | | independent |
| | | | | | | experiments |
| | | Syk KD treated: 1.28 ± 0.19 | scramble treated: 2.03 ± 0.24 | scramble treated to syk | | |
| | | | | knockdown treated | | |
| | | | | p = 0.019 | | |
| Immune cell | 4 | CD95L^{f/+;LysMcre} | CD95L^{f/f} | | | |
| infilration | | | | | | |
| studies | | | | | | |
| 6 h | 4c | Events ± SEM | Events ± SEM | CD45: p = 0.007 | t-test | 4 |
| | | CD45: 653 ± 37 | CD45: 902 ± 34 | Neutrophils: p = 0.025 | | |
| | | Neutrophils: 328 ± 30 | Neutrophils: 460 ± 29 | | | |
| 24 h | 4c | Events ± SEM | Events ± SEM | CD45: p = 0.0022 | t-test | 8 |
| | | CD45: 691 ± 63 | CD45: 1212 ± 118 | Neutrophils: p = 0.025 | | |
| | | Neutrophils: 464 ± 85 | Neutrophils: 815 ± 108 | | | |
| 7 days | 4d | Events ± SEM | Events ± SEM | p = 0.034 | t-test | 3 |
| | | Macrophages: 1341 ± 123 | Macrophages: 2291 ± 273 | | | |
| Acute | | CD95-RB69 | CD95-(R87S)-RB69 | | | |
| neutralization | | | | | | |
| of CD95L | | | | | | |
| 24 h | 4c | Events ± SEM | Events ± SEM | CD45: p = 0.0015 | t-test | 3-5 |
| | | CD45: 698 ± 57 | CD45: 1157 ± 45 | Neutrophils: p = 0.0096 | | |
| | | Neutrophils: 400 ± 54 | Neutrophils: 696 ± 44 | | | |
| Thioglycollate- | 4 | CD95L^{f/+;LysMcre} | CD95L^{f/f} | | | |
| induced | | | | | | |
| peritonitis | | | | | | |
| 2 h | 4f | Neutrophil number ± SEM | Neutrophil number ± SEM | p = 0.19 | t-test | 4 |
| | | 0.61 ± 0.29 *10⁵ | 1.45 ± 0.46 *10⁵ neutrophils | not significant | | |
| 6 h | 4f | Neutrophil number ± SEM | Neutrophil number ± SEM | p = 0.0046 | t-test | 6 |
| | | 11.7 ± 0.51 *10⁵ | 16.91 ± 1.25 *10⁵ | | | |
| 72 h | 4g | Macrophage number ± SEM | Macrophage number ± SEM | p = 0.024 | t-test | 3-5 |
| | | 7.20 ± 1.45 *10⁵ | 15.36 *10⁵ ± 2.07 *10⁵ | | | |
| Acute | | CD95-RB69 | CD95-(R87S)-RB69 | | | |
| neutralization | | | | | | |
| of CD95L | | | | | | |
| 6 h | 4f | Neutrophil number ± SEM | Neutrophil number ± SEM | p = 0.048 | t-test | 4 |
| | | 13.92 ± 1.46 *10⁵ | 19.70 ± 1.86 *10⁵ | | | |
| Thioglycollate- | 4 | wt | tpr | | t-test | 6-7 |
| induced | | | | | | |
| peritonitis | | | | | | |
| | 4h | Macrophage number ± SEM | Macrophage number ± SEM | p = 0.026 | t-test | 6 |
| | | 30.23 ± 7.01 *10⁵ | 10.81 ± 2.45 *10⁵ | | | |
| | 5 | CD95L^{f/+;LysMcre} | CD95L^{f/f} or | | | |
| | | | CD95L^{f/+;LysMcre} | | | |
| CD95L mRNA | 5a | arbitrary units ± SEM | arbitrary units ± SEM | p = 0.009 | t-test | 6 |
| levels | | 0.34 ± 0.21 | 1.91 ± 0.31 | | | |
| Caspase-3 | 5b | pmol/min/μg protein ± SEM | pmol/min/μg protein ± SEM | 3 days: p = 0.38 (not sig.) | t-test | 3 |
| activity | | 3 days: 74.80 ± 8.49 | 3 days: 95.27 ± 19.99 | 7 days: p = 0.00089 | t-test | 4 |
| | | 7 days: 161.79 ± 15.81 | 7 days: 328.14 ± 10.09 | | | |
| Crush-injury | 5c | NeuN counts ± SEM | NeuN counts ± SEM | p = 0.005 | t-test | 6 |
| NeuN counts | | 285 ± 28 | 176 ± 18 | | | |
| Crush-injury | 5d | Distance (μm) ± SEM | Distance (μm) ± SEM | p = 0.004 | t-test | 6 |
| CNPase | | 950 ± 147 | 1760 ± 175 | | | |
| distance | | | | | | |
| Transaction | 5e | Overall improvement | Compared to CD95L^{f/f} | p = 0.015 | Koziol test | 10-12 |
| model BMS | | | Compared to CD95L^{f/+;LysMcre} | p = 0.0072 | | |
| Transection | 5e | Overall improvement | Compared to CD95L^{f/f} | p = 0.021 | Koziol test | 10-12 |
| model | | | Compared to CD95L^{f/+;LysMcre} | p = 0.0057 | | |
| Swimming | | | | | | |
| score | | | | | | |
| Crush-injury | 5f | Overall improvement | | p = 0.0064 | Koziol test | 10-11 |
| BMS | | | | | | |
| Crush-injury | 5f | Overall improvement | | p = 0.0089 | Koziol test | 10-11 |
| Swimming | | | | | | |
| score | | | | | | |
| Microarray | 6c | CD95L^{f/+;LysMcre} | CD95L^{f/f} or | | | |
| validation | | | CD95L^{f/+;LysMcre} | | | |
| | CXCL10 | arbitrary units ± SEM | arbitrary units ± SEM | | | |
| | | 5.06 ± 0.77 | 14.07 ± 2.07 | | | |
| | CCL6 | arbitrary units ± SEM | arbitrary units ± SEM | p = 0.0086 | t-test | 4 |
| | | 5.69 ± 1.28 | 14.36 ± 2.50 | | | |
| | IL1β | arbitrary units ± SEM | arbitrary units ± SEM | p = 0.021 | t-test | 4 |
| | | 12.88 ± 2.40 | 28.23 ± 4.73 | | | |
| | IL6 | arbitrary units ± SEM | arbitrary units ± SEM | p = 0.022 | t-test | 4 |
| | | 6.09 ± 0.97 | 13.91 ± 2.09 | | | |
| | Stat-3 | arbitrary units ± SEM | arbitrary units ± SEM | p = 0.02 | t-test | 7 |
| | | 0.87 ± 0.13 | 1.54 ± 0.25 | | | |
| | S4 | CD95L^{f/+;LysMcre} | CD95L^{f/f} | | | |
| Cre | S4a | arbitrary units ± SEM | arbitrary units ± SEM | p = 0.002 | t-test | 6 |
| recombination | | 0.42 ± 0.04 | 1.00 ± 0.11 | | | |
| In CD11b⁺ cells | | | | | | |
| CD95L mRNA | | | | | | |
| levels | | | | | | |
| Thiogrycollate- | | CD95L^{f/+;LysMcre} | CD95L^{f/f} | | | |
| induced | | | | | | |
| peritonitis | | | | | | |
| 6 h | S4b | arbitrary units ± SEM | arbitrary units ± SEM | p = 0.0001 | t-test | 6-7 |
| Neutrophils | | 0.43 ±0.02 | 1.13 ± 0.12 | | | |
| CD95L mRNA | | | | | | |
| levels | | | | | | |
| 72 h | S4c | arbitrary units ± SEM | arbitrary units ± SEM | p = 0.04 | t-test | 3-5 |
| Macrophages | | 0.35 ± 0.01 | 1.14 ± 0.43 | | | |
| CD95L mRNA | | | | | | |
| levels | | | | | | |
| Apoptosis | S6 | CD95-RB69 | CD95-(R87S)-RB69 | | | |
| levels | | | | | | |
| SCI | S6a | % positive cells ± SEM | % positive cells ± SEM | Not sig. p = 0.24 | t-test | 3-5 |
| | | 9.56 ± 1.19 | 7.07 ± 1.41 | | | |
| Thioglycollate- | S6b | % positive cells ± SEM | % positive cells ± SEM | Not sig. p = 0.529 | t-test | 3-4 |
| induced | | 4.90 ± 0.67 | 4.27 ± 0.60 | | | |
| peritonitis | | | | | | |
| | | CD95L^{f/+;LysMcre} | CD95L^{f/f} | | | |
| Thioglycollate- | S6c | % positive cells ± SEM | % positive cells ± SEM | Not sig. p = 0.734 | t-test | 6 |
| induced | | 6.35 ± 0.86 | 6.03 ± 0.37 | | | |
| peritonitis | | | | | | |
| | S7 | BMT-CD95−/− | BMT-wt | | | |
| CD95L mRNA | S7b | arbitrary units ± SEM | arbitrary units ± SEM | P = 0.029 | t-test | 4 |
| levels | | 0.87 ± 0.26 | 3.62 ± 0.92 | | | |
| Caspase-3 | S7c | pmol/min/μg protein ± SEM | pmol/min/μg protein ± SEM | 3 days: p = 0.24 (not sig.) | t-test | 4 |
| activity | | 3 days: 11.16 ± 2.10 | 3 days: 31.68 ±15.60 | 7 days: p = 0.0015 | t-test | 7 |
| | | 7 days: 138.28 ± 11.88 | 7 days: 211.81 ± 12.16 | | | |
| Crush-injury | S7d | NeuN counts ± SEM | NeuN counts ± SEM | p = 0.04 | t-test | 3-4 |
| NeuN counts | | 253 ± 18 counts | 194 ± 18 counts | | | |
| Crush-injury | S7e | Distance (μm) ± SEM | Distance (μm) ± SEM | p = 0.007 | t-test | 3-4 |
| CNPase | | 750 ± 144 μm | 1500 ± 0 μm | | | |
| distance | | | | | | |
| Transection | S7f | Overall improvement | | p = 0.0029 | Koziol test | 12-13 |
| model BMS | | | | | | |
| Transection | S7f | Overall improvement | | p = 0.0007 | Koziol test | 12-13 |
| model | | | | | | |
| Swimming | | | | | | |
| score | | | | | | |
| Crush-injury | S7g | Overall improvement | | P = 0.0225 | Koziol test | 8 |
| BMS | | | | | | |
| Crush-injury | S7g | Overall improvement | | P = 0.0254 | Koziol test | 8 |
| Swimming | | | | | | |
| score | | | | | | |

### 2.2 Results

### 2.2.1 Injury to the CNS Increases CD95L Surface Expression on Peripheral Blood Cells in Rodents and Humans

We have previously shown that systemic neutralization of CD95L improves functional recovery of spinal injured mice by reducing the number of neurons and oligodendrocytes undergoing apoptosis (Deetjen et al., 2004). Yet, the actual source of CD95L remained elusive. CD95L is poorly expressed in the naïve adult spinal cord and it can be presented by resident spinal cord cells and/or infiltrating leukocytes. To characterize the different populations of immune cells recruited to the injured spinal cord we generated eGFP-bone marrow (BM) chimeras (FIG. 7A). In these mice, every immune cell is eGFP⁺. Using flow cytometry we analyzed the infiltration of immune cells gating on the GFP⁺ cells and using defined cell markers for different immune cell types. Nearly every immune cell infiltrating the lesion site at 24 hours after SCI was CD11b+, a marker for myeloid cells (FIG. 7B,C). From these CD11b+cells, neutrophils accounted for more than 65% (GR-1⁺/F4/80⁻ or GR-1^{high}) and macrophages (CD11b⁺/F480⁺) for 15% (FIG. 7C). Although the CD11b⁺ population remained the most numerous within the first two weeks after injury, the proportion of neutrophils therein rapidly diminished within the first week, when macrophage numbers increased (FIG. 7B). Infiltration of T cells (CD3⁺) started after 7 days (FIG. 7B). In summary, among leukocytes, neutrophils and macrophages are the first to infiltrate the injured spinal cord. In the period of myeloid infiltration, levels of CD95L mRNA and caspase-3 activity reached maximal levels (FIG. 13A,B), suggesting that these cells might represent the major source of CD95L. Indeed, CD95L expression at the surface of peripheral blood neutrophils and monocytes significantly increased 24 hours after SCI (FIG. 7D). Most importantly, increased surface levels of CD95L on peripheral blood neutrophils, were also observed in spinal injured patients at early time points following injury, which returned to control levels at least 1 week following injury (FIG. 7E,F and table below). In patient e, that was followed for 17 days after injury, a second wave of CD95L expression on the surface of neutrophils was observed in the second week post-injury (FIG. 7F). Interestingly, similar upregulation of CD95L on neutrophils was observed in patients with an acute pain episode after spinal disc herniation.

| Patient | Age | Sex | Injury | MP | Surgery |
|---|---|---|---|---|---|
| a | 44 | M | incomplete paraplegia | No | Yes day 1 |
| b | 21 | M | contusio spinalis Th8, | No | No |
| | | | paraplegia | | |
| c | 79 | M | C3/C4 fracture, | No | No |
| | | | complete tetraplegia | | |
| | | | patient died at day 1 | | |
| d | 52 | M | C6/C7 fracture, | Yes | Yes day 1 |
| | | | incomplete tetraplegia | | |
| e | 73 | M | C5/C6 fracture, | No | Yes day 1 |
| | | | complete tetraplegia | | |
| f | 35 | W | spinal disc herniation | No | Yes day 1 |
| g | 56 | M | spinal disc herniation | No | Yes day 1 |
| h | 53 | M | spinal disc herniation | No | Yes day 3 |

### 2.2.2 CD95L Triggers Migration of Neutrophils and Macrophages Through Activation of PI3K and Metalloproteinases via Syk Kinase.

To gain mechanistic insight into the role of CD95L on myeloid cells, we studied the response of myeloid cells to CD95L. The CD95 receptor has been well established as an inducer of apoptosis (Krammer, 2000). Induction of apoptosis via CD95 occurs through the recruitment of the adaptor protein FADD to the DD of the CD95, further leading to activation of caspases. Thus, we first examined FADD association to CD95 on primary macrophages. Yet, CD95L treatment of primary macrophages did not induce a detectable recruitment of FADD to CD95, whereas the same treatment induced efficient recruitment of FADD to CD95 in the CD95-apoptosis sensitive thymoma cell line E20 (FIG. 14). Consistently, macrophages are resistant to CD95-induced cell death (Altemeier et al., 2007; Park et al., 2003; Shimizu et al., 2005). There is increasing evidence that CD95L is involved in processes other than apoptosis. In malignant glioma cells we have recently reported increased migration upon CD95L stimulation (Kleber et al., 2008). In these cells the Src family kinase Yes and the p85 subunit of Phosphatidylinositol-3-Kinase (PI3K) are recruited to CD95 and activated upon CD95L binding. Thereafter, the AKT/8-catenin pathway becomes activated leading to the final induction of MMP-9 expression. The putative YXXL motif in the DD of CD95 was indeed first described in primary neutrophils as a docking site for SH2-containing proteins (Daigle et al., 2002). Besides, activation of PI3K also plays a pivotal role in both survival and migration of neutrophils (Boulven et al., 2006; Zhu et al., 2006). To address whether PI3K is also involved in our system, bone marrow-derived neutrophils and mature macrophages were stimulated with CD95L and phosphorylation of the PI3K target AKT was assessed. Phosphorylation and, thus, activation of AKT was induced upon CD95L treatment in both, neutrophils and macrophages (FIG. 8A,B). Moreover, phosphorylation of Src family kinases (SFKs) also increased upon CD95 stimulation in primary macrophages (FIG. 8B). In order to gain more mechanistical information in neutrophils, we decided to perform further biochemical studies in DMSO-differentiated HL-60 cells (dHL-60), a human neutrophil-like cell line. As in the case of primary macrophages, stimulation of CD95 led to increased phosphorylation of SFKs (FIG. 15).

We next addressed the molecular determinants for PI3K and SFKs activation in immune cells. As the YXXL motif in CD95 was first described in primary neutrophils , we decided to investigate potential CD95 interactors by using an SH2 array (FIG. 8C, upper panel). As shown, CD95, or a CD95-containing multiprotein complex, could interact with the SH2 domain of the non-receptor tyrosine kinase Zap70/Syk (FIG. 8C, lower panel). To validate the results obtained from the protein array, we performed peptide binding experiments, in which the corresponding sequence of CD95 containing the YXXL motif was incubated with CD95L-stimulated or non-stimulated lysates. In dHL-60 cells, incubation of the phosphorylated CD95 peptide resulted in increased binding of Syk compared to the non-phosphorylated CD95 peptide and a scramble phosphorylated peptide used as negative control for sequence specificity (FIG. 8D). Treatment with CD95L further enhanced binding of Syk to the phosphorylated CD95 peptide (FIG. 8D). These results suggest the presence of adapter proteins and/or the requirement of post-translational modifications, which can not be mimicked by the peptide itself. Binding of Syk to the phosphorylated CD95 peptide was also observed in primary macrophages (FIG. 8E). However, contrary to the results obtained in dHL-60, we did not observe differences in the binding upon treatment with CD95L (FIG. 8E). Further, stimulation of CD95 led to increased phosphorylation of Syk in both, dHL-60 and primary macrophages (FIG. 8F and FIG. 15B). In B-cells SFKs get activated by stimulation of the B-cell receptor (BCR) leading to activation of Syk, which can further activate SFKs by phosphorylation of the activation loop, thus, creating a positive feedback loop between both molecules. To analyze possible similarities between CD95 and the BCR we first studied the effect of SFKs on Syk phosphorylation. Inhibition of SFKs with the specific inhibitor PP2 blocked CD95L-induced phosphorylation of Syk in dHL-60 and primary macrophages (FIG. 15B,C). Knockdown of Syk in primary macrophages also abolished CD95-induced phosphorylation of SFKs and AKT (FIG. 8G,H). Taken together, these results reveal that Syk kinase is an upstream activator of PI3K in myeloid cells upon CD95 stimulation.

We next studied CD95L-induced migration of neutrophils and macrophages in vitro in a two-chamber transmigration assay (FIG. 9A-C). Migration of bone marrow-derived murine neutrophils and macrophages and dHL-60 cells significantly increased upon treatment with CD95L (FIG. 9D-F). The increased migration was accompanied by increased activation of the matrix-metalloproteinase-9 (MMP-9) (FIG. 9G-I). Accordingly, pharmacological inhibition of MMP-9 and -2 abolished CD95L-induced migration (FIG. 9J-L). Furthermore, basal migration of primary macrophages was reduced after neutralization of CD95L (FIG. 9M). Thus, exogenous and endogenous CD95L increased macrophage migration in vitro. To address the role of Syk in CD95L-induced migration we knocked down Syk in dHL-60 cells and primary macrophages. Reduced expression of Syk reduced CD95L-induced migration in dHL-60 (FIG. 9N,P) and macrophages (FIG. 9O,Q). Accordingly, knockdown of Syk in primary macrophages abolished CD95L induced MMP-9 activation (FIG. 9R). These data demonstrate that CD95L acts on neutrophils and macrophages in order to increase their migration via Syk (FIG. 9S).

### 2.2.3 CD95L on Myeloid Cells is Involved in Their Recruitment to the Site of Injury In Vivo.

To address if CD95L is also involved in AKT activation in peripheral myeloid cells in vivo, we first analyzed the activation status of AKT after SCI in wt and CD95L-deficient mice. Injury to the spinal cord induced AKT phosphorylation in wt but not CD95L-deficient PBCs (FIG. 10A). To further analyze the role of CD95L in myeloid cells in vivo, we specifically deleted CD95L in neutrophils and macrophages (CD95L^{f/f;LysMcre}). Verified deletion of CD95L in myeloid cells did not influence percentages or absolute numbers of blood leukocytes in naïve or injured animals (FIG. 16). In these mice, we analyzed, as previously described by Stirling and colleagues , the number of immune cells (CD45+) present in the spinal cord following transection injury of the spinal cord (FIG. 10B). In CD95L^{f/f;LysMcre} mice, a significant reduction in infiltrating CD45⁺ cells, largely identified as neutrophils (CD45: GR-1^{high}), was observed (FIG. 10C). Reduced infiltration of neutrophils could already be observed 6 hours after injury, long before the onset of apoptosis (FIG. 10C). Infiltrating monocytes/macrophages (CD45: CD11 b⁺, F4/80⁺) were also markedly reduced 7 days after injury in CD95L^{f/f;LysMcre} mice (FIG. 10D). These data indicate that following SCI, CD95L acts in a paracrine/autocrine fashion on neutrophils and macrophages in order to allow their recruitment to the injured spinal cord. To exclude any possible developmental role of CD95L in neutrophil maturation that could explain their lower infiltration rate into the site of injury, we acutely inhibited CD95L. In previous studies we used neutralizing antibodies to CD95L (Demjen et al., 2004). However, these antibodies greatly varied in their ability to neutralize CD95L. Thus, we generated a stable CD95L-neutralizing CD95 trimer, CD95-RB69, as well as a mutated form, CD95-(R87S)-RB69, which is unable to bind CD95L. Systemic administration of CD95-RB69, but not of the mutated form, decreased the infiltration of neutrophils into the lesion site 24 hours after injury (FIG. 10C). Thus, CD95L on myeloid cells triggers their self-recruitment to the lesion site in vivo.

### 2.2.4 Is the Proinflammatory Effect of CD95L Restricted to the Inflammatory Response Elicited by the Injured CNS?

To address this issue we examined the infiltration of immune cells in an animal model of peritonitis induced by an intraperitoneally injection of thioglycolate (FIG. 10E), a model often used as a mechanistic model for autoimmune diseases. A reduced infiltration of neutrophils into the peritoneum of CD95L^{f/f;LysMcre} mice could already be observed 2 hours after thioglycolate injection (FIG. 10F). Infiltration of neutrophils was significantly reduced 6 hours after thioglycolate injection in CD95L^{f/f;LysMcre} and CD95-RB69-treated animals compared to their respective controls (FIG. 10F). We further assessed infiltration of macrophages in the peritoneum 72 hours after thioglycolate injection. At this time point, CD95L^{f/f;LysMcre} mice showed a lower amount of infiltrating macrophages compared to control littermates, although the number of resident macrophages was not changed (FIG. 10G). mRNA levels of various proinflammatory cytokines were comparable in thioglycolate-elicited cells of control littermates and CD95L^{f/f;LysMcre} mice (FIG. 16G), suggesting that the migratory effect of CD95L is independent of cytokine production. In addition, after thioglycolate activation or SCI, the number of neutrophils undergoing apoptosis was similar in mice lacking CD95L activity and their respective controls (FIG. 17). Consistent with these results, spontaneous death of neutrophils from CD95-deficient lpr (lymphoproliferation) or CD95L-deficient gld (generalized lymphoproliferative disease) mice does not differ from levels in wt mice (Fecho and Cohen, 1998), and blocking CD95/CD95L function with specific antagonists had no effect on the spontaneous death of neutrophils (Brown and Savill, 1999). Thus, CD95L activation of the innate immune response seems to be independent of cytokine production and of CD95L-induced apoptosis. Macrophage recruitment to the inflamed peritoneum after thioglycolate injection was also assessed in lpr mice. In these mice basal numbers of resident macrophages were not changed (FIG. 10H). However, as previously observed in CD95L^{f/f;LysMcre}, 72 hours following thioglycolate injection we could observe a reduced infiltration of macrophages in lpr mice compared to their wt counterparts (FIG. 10H). Accordingly, it has already been shown that thioglycolate-elicited neutrophil response was prolonged in wt mice compared to lpr or gld mice (Fecho and Cohen, 1998).

### 2.2.5 CD95L Acts on the Innate Inflammatory Response to Induce Tissue Damage After SCI

We have demonstrated that CD95L on peripheral myeloid cells is used to facilitate their recruitment to the site of injury/inflammation. Yet, what are the long term consequences of exclusive neutralization of CD95L-induced inflammation? To address this issue, we examined the long term clinical outcome and pathology of spinal injured animals with or without CD95L expression in the immune cell compartment in general or in the myeloid compartment. First, we generated bone marrow transplanted mice (BMT mice) from CD95L-deficient (CD95L^{-/-}) or as a control, from wild-type (wt) donor mice and lethally irradiated wt recipient mice (BMT-CD95L^{-/-} or BMT-wt mice, respectively) (FIG. 18A). CD95L^{-/-} mice could not be used as a recipient due to defects in neuronal development that preclude significant functional recovery following SCI (Demjen et al., 2004; Zuliani et al., 2006). BMT-CD95L^{-/-} mice exhibited a four fold decrease of CD95L mRNA levels and a significantly reduced caspase activity in spinal cord tissue at the time at which injury-induced levels are maximal (FIG. 18B,C). In BMT-CD95L^{-/-} mice, NeuN and CNPase immunoreactivity at 11 weeks after injury was higher compared to BMT-wt mice, indicating that neurons and oligodendrocytes are rescued in BMT-CD95L^{-/-} mice (FIG. 18D,E). These results clearly demonstrate that immune cells are a major source of CD95L following SCI and that the absence of CD95L in the immune cell compartment protects neurons and oligodendrocytes. To assess the long term consequences of CD95L-induced inflammation, BMT-CD95L^{-/-} mice and their respective controls were subjected either to the previously used dorsal 80% transection or to the clinically more relevant crush injury of the spinal cord (Demjen et al., 2004; Plemel et al., 2008). Mice locomotor performance was assessed once weekly over a ten to eleven week period in the swimming test (Demjen et al., 2004) and in the open field using the Basso Mouse Scale (BMS) score (Basso et al., 2006). Following crush injury or transection of the spinal cord, the degree of neurological deficits were significantly reduced in BMT-CD95L^{-/-} mice compared to BMT-wt mice (FIG. 18F,G).

Second, we performed SCI in mice with exclusive deletion of CD95L in neutrophils and macrophages (CD95L^{f/f;LysMcre}) and their control littermates. Importantly, after transection injury, spinal cord CD95L mRNA levels were highly reduced in CD95L^{f/f;LysMcre} mice 24 hours after injury, further demonstrating that infiltrating myeloid cells are the major source of CD95L (FIG. 11A). Besides, 3 days after injury, caspase-3 activity in the spinal cord of CD95L^{f/f;LysMcre} mice was lower than in control littermates, reaching significance at 7 days (FIG. 11B). Consistently, 11 weeks after injury, CD95L^{f/f;LysMcre} mice had an increased number of surviving neurons and oligodendrocytes compared to their respective controls (FIG. 11C,D). Furthermore, deletion of CD95L in the myeloid compartment allowed for a higher functional recovery following either crush or transection injury to the spinal cord in the BMS as well as in the swimming test (FIG. 11E,F). To analyze a possible effect of T cell-derived CD95L, CD95L^{f/f;LysMcre} mice and control littermates underwent crush injury to the spinal cord. Contrary to CD95L^{f/f;LysMcre} mice, SCI-induced neurological deficits were comparable in CD95L^{f/f;LCKcre} and their respective controls (FIG. 19). Thus, we clearly identified neutrophils and macrophages as the major source of CD95L, inducing death of neurons and oligodendrocytes and, therefore, participating in the pathogenesis of SCI.

### 2.2.6 Characterization of the Inflammatory Environment After Neutralization of CD95L

Neutralization of CD95L reduces infiltration of neutrophils and macrophages into the injured spinal cord leading to a long term recovery of the locomotor function. Thus, regulation of inflammation upon neutralization of CD95L on myeloid cells creates a controlled inflammatory response that facilitates functional recovery of spinal injured animals. In order to characterize the molecular events regulated upon neutralization of CD95L on myeloid cells, we examined the gene signature of CD95L^{f/f;LysMcre} mice and their littermate counterparts in the spinal cord 24 hours after transection injury. Already at this early time point, regenerative processes including organogenesis, development and neurogenesis are switched on in CD95L^{f/f;LysMcre} mice (FIG. 20A). Similarly, expression of genes involved in apoptosis was reduced in CD95L^{f/f;LysMcre} mice or CD95-RB69 treated mice as compared to their respective controls (FIG. 12A,B). Beyond this, lack of CD95L in myeloid cells or neutralization of CD95L in CD95-RB69 treated animals resulted in down-regulation of genes involved in the immune response (FIG. 12A,B). The observed down-regulation of proinflammatory genes was further validated by qRT-PCR (FIG. 12C and data not shown). Importantly, among these downregulated proinflammatory cytokines, neutralization of IL-6, IL-1 or CXCL10 is reported to improve functional recovery after SCI (Akuzawa et al., 2008; Gonzalez et al., 2007; Okada et al., 2004). Interestingly, 24 hours after SCI, 65.2% of genes were commonly regulated between the group with genetic deletion of CD95L on myeloid cells (CD95L^{f/f;LysMcre} vs. CD95L^{f/f} littermates) and the group with pharmacological inhibition of CD95L (CD95-RB69-treated vs. vehicle-treated mice), indicating that at this time point the gene signature is due to the exclusive deletion of CD95L in the immune cell compartment (FIG. 12D). Further, we compared the datasets from following animals and their respective control counterparts 24 hours following SCI: CD95L^{f/f;LysMcre}, CD95L^{-/-} and CD95-RB69-treated mice. As opposed to CD95L^{f/f;LysMcre} animals, in the latter two groups CD95L from resident spinal cells is also targeted. For statistically-based meta-analysis of combined microarray of our three microarray datasets, the GeneMeta package from Bioconductor (http://bioconductor.orq) was applied. This analysis provides a single estimate of the degree of differential expression for each gene, while simultaneously accounting for the detection of differences between each experiment and animal background. Comparison of these three datasets allowed the detection of 612 genes that were consistently and significantly differentially regulated in the spinal cord 24 hours after injury (FIG. 12E and FIG. 20B). The identification of a common gene signature regardless of the site of CD95L inhibition implies that the initial cause of CD95L-induced damage is the activation of the innate inflammatory response.

To finally assess the contribution to tissue damage of CD95L-induced inflammation vs. direct CD95L-induced apoptosis, we examined caspase activity in mice deficient of CD95 in resident neural cells (CD95^{f/f;NesCre}) and their littermate controls (CD95^{f/f}). The extent of caspase-3 activity did not differ between the two groups (FIG. 12F and FIG. 21). This data indicates that CD95L detrimental function following SCI is rather due to its influence on the innate inflammatory response than to direct apoptosis of CD95-bearing resident neural spinal cells.

### 2.2.7 Discussion

Our results reveal a novel mechanism by which CD95L/CD95 on myeloid cells mediates their recruitment to the inflammatory site via the Syk/AKT/MMP pathway. We show that an injury to the CNS increases expression of the CD95L/CD95 system on myeloid cells in rodents and humans. This system is also involved in the recruitment of myeloid cells to the inflamed peritoneum after thioglycolate injection. Further, we show that neutralization of CD95L reduces the initial infiltration of inflammatory cells creating an inflammatory response that facilitates recovery of locomotor function after SCI.

### CD95L: a Mediator of Inflammation

Until the mid 90's the dogma that apoptosis does not induce inflammation was strongly anchored in the scientific community. It was generally believed that CD95L resolves inflammation by inducing activation-induced-cell-death (AICD) of T cells (Griffith et al., 1995; Griffith et al., 1996; Nagata, 1999). Along this line, constitutive expression of CD95L by cells in the eye and testis was thought to contribute to the immune-privileged status of these organs (Griffith et al., 1995; Griffith et al., 1996). It was further suggested that constitutive CD95L expression by a variety of tumor populations would lead to immune evasion (Hahne et al., 1996; O'Connell et al., 1996; Strand et al., 1996). Regarding these findings, researchers postulated that forced expression of CD95L might effectively protect allografts from rejection. Unexpectedly, most cell types and tissues genetically engineered to express CD95L undergo destruction through neutrophils (Allison et al., 1997; Kang et al., 1997; Seino et al., 1997). This data would indicate a role for CD95L as a chemoattractant. Alternatively, it is known that CD95L is quickly removed from the surface of the cell by metalloproteinases and the released CD95L to the blood can bind to CD95 on peripheral myeloid cells and trigger their recruitment—in this case the engineered tissue. Indirect evidence for a similar role of CD95L in autoimmune disease is given by the fact that the lpr mutation ameliorates disease signs in mice with experimental autoimmune encephalomyelitis and collagen-induced arthritis (Hoang et al., 2004; Ma et al., 2004; Sabelko et al., 1997). Accordingly, in the inflamed peritoneum the recruitment of macrophages was lower in lpr animals than in their control counterpart. However, the basal lymphoproliferative disease resulting from the lpr mutation hampers the study of inflammation on this strain and can only be addressed by the conditional ablation of the CD95/CD95L on specific subsets of inflammatory cells. Here we show that exclusive deletion of is CD95L on myeloid cells ameliorates the innate inflammatory response in an animal model of peritonitis and of spinal cord injury. Accordingly, proinflammatory cytokines and chemokines such as IL-1β, IL-6, CXCL10 and CCL6 were down-regulated in the injured spinal cord of mice lacking CD95L in myeloid cells as compared to their control counterparts. Most of the proinflammatory cytokines are reported to impair axonal conduction and to amplify the inflammatory response following injury, thus further inducing tissue damage (Schnell et al., 1999; Yang et al., 2004). Consistently, neutralization of IL-6, IL-1 or CXCL10 is reported to improve functional recovery after SCI (Akuzawa et al., 2008; Gonzalez et al., 2007; Okada et al., 2004).

Former strategies to study the role of circulating neutrophils, dealing with their depletion, inhibition of neutrophil-related proteolytic enzyme activities or inhibition of neutrophil adhesion did not lead to a full ablation of neutrophilic function and resulted in improved motor recovery of spinal cord injured mice (Trivedi et al., 2006). A recent study showing full depletion of neutrophils via the Ly6/Gr1 antibody prior to SCI reports increased levels of several proinflammatory cytokines including IL-6 and a worsened clinical outcome following SCI of depleted animals (Stirling et al., 2009). Thus, it seems that a complete abrogation of neutrophils amplifies the inflammatory response. It is noteworthy, that neutrophils and macrophages not only contribute to tissue damage but also play an important role in cleaning the injury site, limiting bacterial infection and promoting wound healing. In our study, neutralization of CD95L led to a reduction without complete abrogation of infiltrating neutrophils and macrophages. Whether the dose of resulting inflammation is beneficial or rather the fact of having inflammatory cells without CD95L remains subject of future studies. At least, since mice with exclusive deletion of CD95 in neural cells were not protected from apoptosis, it seems that CD95L on infiltrating inflammatory cells does not have an additional role on direct induction of apoptosis of CD95-bearing cells.

### CD95 Signals Inflammation via the SYK/PI3K/MMP Pathway

We have previously shown that CD95L triggers invasion in a glioblastoma model via the PI3K/β-catenin/MMP pathway (Kleber et al., 2008). In primary neutrophils and macrophages, CD95 stimulation led to phosphorylation of AKT, activation of MMP-9 and, ultimately, increased migration. Pharmacological inhibition of MMP-2 and MMP-9 blocked migration triggered by CD95L, demonstrating that MMPs are crucial for CD95L-induced migration. In primary macrophages blocking of CD95L by neutralizing antibodies led to a reduced basal migration, pointing out that CD95L is needed for migration of these cells. But how does CD95 induce PI3K activation? In 1996, Atkinson and colleagues reported for the first time a physical interaction between CD95 and a non-receptor tyrosine kinase, the Src family member Fyn in T cells (Atkinson et al., 1996). They further described the presence of a highly conserved tyrosine-containing YXXL motif located in the death domain of CD95 that resembles an Immunoreceptor-Tyrosine-Activation-Motif (ITAM). Six years later, Daigle and colleagues (Daigle et al., 2002) showed that stimulation of CD95 in primary neutrophils leads to phosphorylation of this motif, thus serving as docking sites for SH2-domain containing proteins. Phosphorylation of the receptor is thought to be driven by members of the Src family of nonreceptor tyrosine kinases (SFKs: Src, Fyn, Yes, Lck, Hck and Lyn) (Atkinson et al., 1996). Once the YXXL motif is phosphorylated, other SH2-containing protein kinases or phosphatases could potentially bind and initiate activation of downstream signaling pathways. Here, we show that CD95L stimulation of CD95 on myeloid cells activates Syk, further leading to PI3K/MMP signaling. Accordingly, blocking PI3K or Syk has been shown to inhibit migration of immune cells (Ali et al., 2004; Boulven et al., 2006; Frommhold et al., 2007; Schymeinsky et al., 2007). This finding may have broader implications. Syk is known as an important activator of inflammatory responses by ITAM-coupled activated receptors, the inflammatory response mediated by proinflammatory crystals and activation of the inflammasome (Gross et al., 2009; Schymeinsky et al., 2006). Recently, Syk inhibitors have shown beneficial clinical effects in inflammatory disorders, which might at least in part, involve the CD95 receptor (Pine et al., 2007; Weinblatt et al., 2008).

### CD95-Induced Apoptosis Versus CD95-Induced Inflammation

While regulation of cell death is one of the best known functions of CD95, it is also capable of activating signal transduction pathways leading to the induction of proinflammatory responses (Baud and Karin, 2001). Pre-apoptotic macrophages and neutrophils can release proinflammatory cytokines, like MCP-1 and IL-8, which participate in the induction of the inflammatory response. Hohlbaum and colleagues indicated that preapoptotic peritoneal macrophages produce MIP-2, IL-16, MIP-1α, MIP-1β, followed by neutrophil extravasation (Hohlbaum et al., 2001). However, after thioglycolate activation or SCI, the number of neutrophils undergoing apoptosis was similar in mice lacking CD95L activity and their respective controls. Furthermore, resident numbers of peritoneal macrophages were not changed between mice lacking CD95L in myeloid cells and their controls. Thus, CD95L activation of the innate immune response seems to be independent of CD95L-induced apoptosis.

Do neurons and oligodendrocytes die in the injured spinal cord due to direct CD95-induced death or rather to CD95-elicited inflammation? It has been shown that neutrophils can kill bystander cells in co-culture systems through the CD95 system (Brown and Savill, 1999; Serrao et al., 2001). Further, phagocytosis triggers macrophage release of CD95L and, thus, is able to induce cell death of bystander cells. In addition, a recent study from Michael Fehlings group demonstrated that CD95L is directly able to induce death of oligodendrocytes through both intrinsic and extrinsic pathways of the CD95-mediated apoptotic signaling (Austin and Fehlings, 2008). However, all these data have been provided by in vitro studies. To correctly address this question in vivo, we specifically deleted the CD95 receptor in the CNS resident neural cells during embryonic development and assessed caspase-3 activity after SCI. Interestingly, CD95 expression in the CNS compartment does not seem to influence the apoptosis levels in the injured spinal cord. In addition comparison of the gene signature of spinal injured animals with either pharmacological, ubiquitous or exclusive inhibition of CD95L in the myeloid compartment revealed a high degree of similarity that indicates that, at least within the acute phase following SCI, the main role of CD95L is induction of inflammation. Altogether, these data suggest that CD95L rather kills neurons and oligodendrocytes through an inflammation-induced mechanism and not as previously thought through a direct apoptosis mechanism. As a consequence, neutralizing agents to CD95L do not have to be administered locally in the CNS but can be systemically applied directly after injury by paramedics. Beyond this, neutralization of CD95/CD95L system appears as a candidate therapy for inflammatory diseases in general.

### 3. Identification of Significant Genes by Meta-Analysis

To detect significant differential expression of a gene between animals with or without CD95L inhibition across studies that used different modes of action, we applied a meta-analysis approach as described by Choi and colleagues (Choi et al., 2003). For each gene in every study i, the standardized mean difference between animals with CD95L inhibition and those of the control group was calculated as an effect size dᵢ=(Xₐᵢ−X_{ci})/Sₚᵢ, where Xₐᵢ and X_{ci} represent the means of the group of animals with CD95L inhibition or of the control group, respectively, and Sₚᵢ is the pooled standard deviation. A test statistic Q was used to decide whether a fixed effects model (FEM) or a random effects model (REM) is more appropriate to combine the effect sizes of the different studies. A FEM assumes that the effect sizes (here, the standardized mean differences) observed in the different studies are samples of the same distribution. A REM explicitly accounts for differences between the studies by postulating that each effect size is drawn from a distribution with study-specific parameters. Under the assumption that the differences in the effect sizes between studies is due to sampling error alone, the values for Q are distributed according to a X² distribution. Upon inspection of the distribution of Q, it was decided that a REM would be more appropriate (data not shown).

Study-specific effect sizes were then combined in order to estimate the average effect size as described by Choi and colleagues (Choi et al., 2003). Genes were chosen by comparing the effect size estimates with a given threshold and estimating the statistical significance with the concept of false discovery rate (FDR) based on empirical null distributions generated by random permutations (Choi et al., 2003).

### 4. Additional Information on Materials and Methods

### 4.1 Reagents and Antibodies

We purchased RPMI 1640 medium (#21875-091), penicillin/streptomycin (#15140-163), L-Glutamin (#25030024) and 55 μM 1′-Mercaptoethanol (#31350) from Invitrogen, Karlsruhe, Germany. Fetal Calf Serum (FCS, #S0115) was purchased from Biochrom, Berlin, Germany.

The following antibodies were used for flow cytometry experiments: Fitc-conjugated anti-mouse Ly6G mAb (BD #551460), PE-conjugated anti-mouse F4/80 mAb (Caltag #MF48004), PE-conjugated rat IgG2a mAB (isotype control, BD #553930), PercP-Cy5-conjugated anti-mouse CD45.2 mAb (BD #552950), Fitc-conjugated anti-mouse CD45.1 mAb (BD #553775), PeCy7- or APC-conjugated anti-mouse CD3 mAb (BD, APC #553066, PeCy7 #552774), Alexa-680- or APC-conjugated anti-mouse CD11b mAb (BD, Alexa 680 #RM2829, APC #553312), APC-conjugated anti-mouse GR-1 mAb (BD #553129), APC-Cy7-conjugated anti-mouse CD19 mAb (BD #557655), biotin-conjugated anti-mouse CD95L mAb (BD #555292), biotin-conjugated hamster IgG mAb (isotype control, BD #553970), streptavidin-APC (BD #349024, 1:50), mouse anti-human CD95L (NOK-2, BD #556375), anti-mouse APC (BD #550826), mouse IgG2K (Acris #AM03096PU-N), Fitc-conjugated anti-human CD66b (BD #555724), PE-conjugated pAKT (BD #560378) and PE-conjugated IgG (BD #554680). Unless otherwise indicated, all antibodies from BD were used at a dilution of 1:100.

### 4.2 Bone Marrow Transplantation

Recipient mice (4-6 week old) carrying the congenic marker CD45.1 were lethally irradiated with 450 rad 2 times at 3 h intervals in order to deplete their own bone marrow (BM). Bone marrow cells (BMCs) were isolated from the femur and tibia of either male mice that ubiquitously express an enhanced green fluorescent protein or wt and CD95L^{-/-} female mice carrying the congenic marker CD45.2. Three hours after the last irradiation, recipient mice were injected in the tail vein with 4−6×10⁶ cells. Mice were kept in a specific pathogen-free facility and were given drinking water containing amoxicillin (1 mg/ml) to prevent infections. Eight weeks after transplantation, bone marrow reconstitution was checked by flow cytometry using antibodies against CD45.1 and 2 as well as antibodies for the different immune cell populations. Mice with lower reconstitution than 90% were excluded from further studies.

### 4.3 Flow Cytometry

Stainings were performed on cells derived from bone marrow, peritoneum, blood or spinal cord tissue. For preparation of mouse cells derived from spinal cord tissue, the animals were perfused with HBSS to remove blood from the organs. Then the spinal cord (1 cm around the lesion site) was isolated and lysed for 3 h in thermolysin (0.5 mg/ml, Sigma #T-7902) on a shaker at 37° C. Tissue was incubated for 10 more minutes in trypsin 0.5%-EDTA (Invitrogen #25300096) and finally homogenized by passing 10 times through a Pasteur pipette and through a 40 μm cell strainer (BD #352340).

The staining was performed on this homogenized fraction.

For all stainings, cells were resuspended in FACS buffer (PBS, 0.2% NaN₃) and preincubated in Fc block for 10 minutes before stained with the respective antibodies 30 minutes on ice. For intracellular stainings blood samples were fixed with 4% PFA after Ery Lysis and permeabilized with methanol before the staining. Samples were run on a FACSCantoll flow cytometer (BD) and analyzed using FACSDiva (BD) software or FlowJo software. For all FACS analyses done on cells derived from spinal cord tissue 1,000,000 events were counted.

### 4.4 Immune Cell Type Identification

For all tissue analysis, neutrophils were identified as CD45 positive, GR-1 high-positive and their characteristic forward (FSC) and side scatter (SSC) profile. Macrophages were identified as CD45 high-positive, CD11b positive is and F4/80 positive. In the time kinetic analysis, all immune cell types were identified by the same marker as described in this paragraph. However, hematopoietic cells in the eGFP BMT mice were GFP positive and therefore, appeared in the FITC channel without any prior antibody staining contrary to all other studies in which they were followed by CD45 positivity. T cells were identified as CD3 positive. Resident microglia are also known to express CD45 at low levels. However, we could not find any sign of cre recombination in the microglia population of the LysMcre line (data not shown), indicating that this cell population would not primarily be affected. In addition, detection of CD45 by flow cytometry enabled the distinction between CNS-resident microglia (CD45 low) and infiltrating macrophages (CD45 high).

Concerning the cells derived from the bone marrow or from the thioglycolate-induced peritonitis, we used the Ly6G mAb to characterize neutrophils.

### 4.5 Processing Spinal Cord Tissue

At the described time points after surgery, animals were deeply anesthetized with an overdose of Rompun and Ketanest intra-peritoneally (i.p.) and killed by transcardial perfusion with HBSS (for RNA and protein and tissue extraction) or HBSS and 4% PFA (for immune-histochemistry and fluorescence). Depending on the experiment, 0.5 cm (caspase-3 activity assay), 1 cm (infiltration assays) or 2.5 cm segments (microarrays) around the lesion site were extracted.

### 4.6 Thioglycolate-Induced Peritonitis

For thioglycolate-induced peritonitis, 1 ml of 3% thioglycolate broth (Fluka #70157) was injected i.p. in CD95L^{f/f;LysMcre} and CD95L^{f/f} mice or in wt mice acutely treated with CD95-RB69 or its respective control. In this model, neutrophils are known to start infiltrating the peritoneum within the first hours, whereas macrophage infiltration peaks at 72 h. At the indicated times, mice were sacrificed, blood samples collected and peritoneal cavities lavaged with 10 ml sterile Hanks balanced salt solution (HBSS; Invitrogen #14170-138) containing 0.25% bovine serum albumin (Roche #10735094001). Total cell counts were performed in a Neubauer hematocytometer (Brand), and differential cell counts were carried out by flow cytometry. Results are expressed as the absolute number of neutrophils or macrophages×10⁵/cavity. For every experiment performed, blood immune cell populations were analyzed by the appropriate cell markers.

### 4.7 Gelatin Zymography for Activated MMPs

MMP activity in cell-free supernatants from neutrophils, dHL-60 or macrophages treated with different doses of CD95L-T4 was determined by gelatinase zymography as described previously. In brief, neutrophils were treated with CD95L-T4 (10 and 20 ng/ml) for 6 h, dHL-60 with CD95L-T4 (10, 20 and 40 ng/ml) for 6 h, and macrophages with CD95L-T4 (10, 20 and 40 ng/ml) for 24 h. After electrophoresis and washing the gel with Triton X-100 (2.5% v/v, twice for 30 minutes)(Sigma #X-100), the gel was incubated in MMP reaction buffer [50 mmol/L Tris-HCl (pH 7.8), 200 mmol/L NaCl, 5 mmol/L CaCl2] at 37° C. for 16 h. Gelatinolytic activity was detected as transparent bands on staining with Coomassie Brilliant Blue G-250 solution and incubation in destaining solution (10% acetic acid, 20% methanol). Data are representative of at least 2 independent experiments.

### 4.8 Analysis of Apoptotic Cells by Annexin-V Staining

Annexin-V staining was performed on the neutrophil population either from the peritoneal exudates or from the injured spinal cord. After gating on the neutrophil population using appropriate markers and characteristic FSC and SSC, the percentage of annexin-V positive cells was determined by using a phycoerythrin-conjugated annexin-V according to the manufacturer's protocol (Calbiochem # CBA060).

### 4.9 Isolation and Culturing of Murine Neutrophils

Bone marrow neutrophils were isolated from the femur of mice by flushing the bones with PBS/2 mM EDTA. Harvested bone marrow cells were resuspended in ACK buffer (150 mM NH4Cl, 10 mM KHCO3, 1 mM Na2EDTA, pH 7.3) and incubated for 1 min to lyse erythrocytes. Neutrophil selection was performed using MACS-positive selection by magnetic beads according to the manufacturer's protocol (Miltenyi, #130-092-332). Neutrophils were given in culture medium and left for 2 h until used for further experiments (RPMI 1640 supplemented with 1% penicillin/streptomycin, 0.1% 55 μM β-mercaptoethanol, 10% FCS, 1% L-glutamine, 10 mM Hepes, 1% non-essential amino-acids, 1% sodium pyruvate). Purity of neutrophils was assessed by FACS and reached >96%. In vivo activated neutrophils were isolated by washing the peritoneal cavity of mice 6 h after the injection of 3% thioglycolate.

4.10 Cell Isolation of CD11b⁺ Cells

Bone marrow cells were isolated as previously described. CD11b selection was performed according to the manufacturer's protocol (Miltenyi #130-092-333).

### 4.11 Primary Cell Culture and Transfection of Macrophages

To obtain bone marrow-derived macrophages (BMDM), femurs and tibias were harvested bilaterally and marrow cores were flushed using syringes filled with PBS/2 mM EDTA. Cells were triturated and red blood cells were lysed using the ACK buffer. After washing once in media, the cells were plated and cultured in RPMI 1640 supplemented with 1% penicillin/streptomycin, 0.1% 5.5 μM β-mercaptoethanol, 10% FCS, 1% L-glutamine, 1% non essential amino-acids, 1% sodium pyruvate and 20% supernatant from macrophage colony stimulating factor secreting L929 cells (sL929; a kind gift from Dr. Tobias Haas). The sL929 drives bone marrow cells towards a macrophage phenotype (7-10 days). At day 1 non-adherent cells were collected and further cultivated. 4 days later fresh medium was added to boost the cell growth. At harvest, 95±0.7% of cells were macrophages (assessed by CD11b and F4/80 immunostaining). Supplemented culture media was replaced with RPMI/10% FCS on the day of stimulation so that stimulations were performed in the same media for all cell types.

Transfection of primary macrophages was performed at day 8 in culture with lipofectamine (Invitrogen #11668019) according to the manufacturer's protocol. Briefly, macrophages were transfected with mouse 600 μmol Syk siRNA ON-TARGETplus SMARTpool siRNA or a non-targeting SMARTpool siRNA using Lipofectamine 2000. 48 h later Syk knockdown was assessed by Western Blot. At the same time, cells were stimulated with CD95L-T4 and analysed after 24 h for migration, MMP-activity or Western blots.

4.12 Cell Culture and Transfection of dHL-60 Cells

The human myeloid HL-60 cell line (ACC 3) was kindly provided by Dr. Lucie Darner. PMN-like differentiation of HL-60 cells and the electroporation protocol was described previously. Briefly, HL-60 cells were allowed to differentiate in presence of 1.3% DMSO for 6 days before used for protein analysis. Electroporation of dHL-60 cells was performed at day 4. For electroporation, a 400 μL aliquot of dHL-60 (1×10⁷ cells/mL) in RPMI was transferred to a Gene Pulser cuvette with an 0.4-cm electrode (Bio-Rad, Hercules, Calif.) and mixed with 600 μmol Syk siRNA ON-TARGETplus SMARTpool siRNA or non-targeting SMARTpool siRNA. Cells were incubated for 10 minutes at room temperature (RT) and subjected to an electroporation pulse of 310 V and 1175 μFF (Gene Pulser Biorad, Munich, Germany). 48 h to 72 h after electroporation, Syk knockdown was assessed by Western Blot. At the same time, cells were stimulated with CD95L-T4 and analysed after 4 h for migration.

### 4.13 SH2 Array

The Transsignal SH2 Domain Array (Panomics) was performed according to the manufacturers instructions. For hybridisation of whole cell lysates, cells were harvested as described above. Lysates were then incubated with 5 μg anti-CD95 antibody Jo2—biotin and subsequently hybridised to the SH2-array membrane. After washing the array was incubated with streptavidin-HRP and developed.

### 4.14 Western Blots

Protein extraction and immunoblotting was performed as previously described. Membranes were probed with the following antibodies: phosphorylated AKT (p-Ser473-AKT, 1:1000, Cell Signaling #9271), total AKT (t-AKT, 1:1000, Cell Signaling #9272), phosphorylated Src (p-Src Tyr 416, 1:1000, Cell Signaling #2101), total Src (1:1000, Cell Signaling #2108), phosphorylated Syk (pSyk Tyr 319/352, 1:1000, Cell Signaling #2701), total Syk (1:1000, Cell Signaling #2712).

### 4.15 Immunoprecipitation

At least 1×10⁷ cells were treated with 10 (neutrophils) or 20 (macrophages) ng/ml of mCD95L-T4 for 5 minutes at 37° C. or left untreated, washed twice in PBS plus phosphatase inhibitors (NaF, NaN3, pNPP, NaPPi, II-Glycerolphosphate, 10 mM each and 1 mM orthovanadate), and subsequently lysed in buffer A [(20 mM Tris/HCl, pH 7.5, 150 mM NaCl, 2 mM EDTA, 1 mM phenylmethylsulfonyl fluoride, protease inhibitor cocktail (Roche #11836145001), 1% Triton X-100 (Sigma, X-100), 10% glycerol, and phosphatase inhibitors (NaF, NaN3, pNPP, NaPPi, β-Glycerolphosphate, 10 mM each and 1 mM orthovanadate)]. Protein concentration was determined using BCA kit (Pierce #23225). 500 μg of protein was immunoprecipitated overnight with either 5 μg anti-CD95 Ab Jo2 (BD #554255) and 40 μl protein-A Sepharose (Sigma #P3391) or the corresponding isotype control (BD #554709). Beads were washed 5 times with 20 volumes of lysis buffer. The immunoprecipitates were mixed with 50 μl of 2× Laemmli buffer and analyzed on 15% SDS-PAGE. Subsequently, the gels were transferred to Hybond nitrocellulose membrane (Amersham Pharmacia Biotech #RPN203D), blocked with 5% milk in PBS/Tween (PBS plus 0.05% Tween 20) for 1 h, and incubated with the primary antibody in 5% milk in PBS/Tween at 4° C. overnight. Blots were developed with a chemoluminescence method following the manufacturer's protocol (PerkinElmer Life Sciences, Rodgan, Germany). The highly CD95L-sensitive mouse thymoma cells (E20), kindly provided by Dr. Mareike Becker, were included as a positive control for analysing FADD recruitment (anti-FADD mouse monoclonal Ab, clone 1F7, Millipore #05-486).

### 4.16 Peptide Competition Experiments

Biotinylated peptides including CD95-tyrosine 283 in their phosphorylated and non-phosphorylated forms as well as scramble peptides were produced by the DKFZ Peptide Synthesis facility. Briefly, 50 μM peptides were incubated with 500 μg of total protein lysates overnight at 4° to allow displacement and binding by molarity competition with endogenous protein complexes. The formed peptide-protein complexes were precipitated with 40 μl monomeric avidin beads (Thermo Scientific, #20228) for 1-2 hours at 4° and washed five times with 1 ml IP lysis buffer. After washing, beads were resuspended in 40 μl of 2× Laemmli buffer and the precipitates were analysed by SDS-PAGE and Western blotting.

### 4.17 Caspase-3 Like Activity Assay

To determine caspase-3 activity after SCI, the spinal cord (0.5 cm around the lesion site) was dissected and homogenized in 10 times the volume of lysis buffer (250 mM HEPES, 50 mM MgCl₂, 10 mM EGTA, 5% Triton-X-100, 100 mM DTT, 10 mM AEBSF, pH 7.5). Samples were centrifuged for 10 minutes at 12,000 g. Apoptosis is paralleled by an increased activity of caspase-3. Hence, cleavage of the specific caspase substrate Ac-DEVD-AFC (Biomol) was used to determine the extent of apoptosis. Ac-DEVD-AFC can be cleaved by several caspases, however, caspase-3, -7 and -8 display by far the strongest specificity for this substrate.

For the Caspase activity assay, 20 μl cell lysate were transferred to a black 96-well microtiterplate. After the addition of 80 μl buffer containing 50 mM HEPES, 1% Sucrose, 0.1% CHAPS, 50 μM Ac-DEVD-AFC, and 25 mM DTT, pH 7.5, the plate was transferred to a Tecan Infinite F500 microtiterplate reader and the increase in fluorescence intensity was monitored (excitation wavelength 400 nm, emission wavelength 505 nm). The substrate cleavage of the samples is quantitatively determined by using an AFC standard curve. The results are expressed in pmol/min/μg protein.

### 4.18 Migration Assay

Migration of bone marrow derived neutrophils or macrophages was assessed in vitro in a two chamber migration assay. Transwell inserts [3 μm (BD #353096) or 8 μm (BD #353097) pore size for neutrophils or macrophages, respectively] were coated with matrigel (50 μg/ml; BD #354234). 5×10⁵ neutrophils, 1×10⁶ dHL60 or 2×10⁵ macrophages were plated in 500 μl medium onto the upper chamber. Cells were left untreated or treated with CD95L-T4 (engineered Mus musculus CD95L (Kleber et al., 2008)) by adding 10, 20 and 40 ng/ml to the upper chamber. The number of migrated cells was counted 3 h for neutrophils, 4 h for dHL-60 and 24 h for macrophages after treatment by using a hemocytometer. CD95L-induced migration of macrophages was analyzed by blocking basal migration of macrophages by using neutralizing antibodies to CD95L (MFL3, 10 μg; BD #555290) or the appropriate isotype control (IgG, 10 μg; BD #554709). Data of the migration assays are representative of at least 4 independent experiments with 6 technical replicates per condition.

The role of metalloproteinases on neutrophil and macrophage recruitment was investigated by using selective inhibitors of MMP-2/9. Neutrophils, dHL-60 and macrophages were pre-incubated with MMP-2/9 inhibitors (50 μM; Calbiochem #444251) 30 minutes prior to CD95L-T4 treatment and the number of migrated cells was counted at the times indicated previously.

### 4.19 Tissue Processing, Immunohistochemistry and Quantification

Depending on the experiment, mice were transcardially perfused 9-11 weeks following SCI using HBSS and 4% paraformaldehyde (PFA). Spinal cords were dissected, post-fixed overnight at 4° C. in 4% PFA and processed for paraffin embedding. Paraffin blocks were mounted on a microtome and cut into 8-10 μm transverse sections. For immunohistochemistry, sections were permeabilized with 0.2% Triton-X 100 at RT and blocking of unspecific binding was performed using serum. After staining, slides were coverslipped with Mowiol, dried overnight at RT and stored at 4° C. until they were analyzed with an Olympus microscope. In all immunohistochemistry stainings, one slide was used as a negative control to assess non-specific binding. For neuron and oligodendrocyte labeling, slides were incubated with the primary antibody at 4° C. overnight followed by a fluorescent labeled secondary antibody (1 h at RT). Primary antibodies used were anti-NeuN (mouse, 1:200; Chemicon #MAB377) and anti-CNPase (mouse, 1:200; Sigma #C5922), respectively. Secondary antibody used was donkey anti-mouse rhodamine X (1:200; Dianova #715-296-150). To label the nuclei, Dapi (Sigma #D9564) 1:3000 was used. In order to quantify neurons, images were taken at the epicenter of injury and every 350 μm until reaching 1500 μm rostral and caudal to the epicenter and NeuN positive cells were counted in mice 10-11 weeks after SCI. The mean of NeuN positive cells per slide is presented. In order to quantify oligodendrocytes, CNPase stainings of tissue sections taken every 350 μm rostral and caudal to the lesion site were analyzed. Analysis was performed by determining the distance between the lost CNPase signal rostral and the reappearance of the CNPase staining caudal to the lesion site in the dorsal funiculus of the spinal cord. The distance indicates the level of white matter sparing in the spinal cord. A shorter distance correlates with a higher white matter sparing.

### 4.20 Isolation of RNA, Real-Time Quantitative PCR and Microarrays

For tissue, spinal cords were dissected out and RNA was extracted with the mirVana microRNA Extraction Kit essentially according to the manufacturer's protocol (Ambion #AM1560). mRNA of injured mice were represented as normalized to the respective uninjured animals. Cells from peritoneal exudates or bone-marrow derived cells were washed with PBS and taken up in RLT-buffer containing β-mercaptoethanol. RNA was extracted using the RNeasy Mini Kit (Qiagen, #74104).

In all cases, real-time quantitative PCR was carried out using Sybr Green core kits (Eurogentec) and Uracil-N-glycosylase (Eurogentec). Primers used for quantitative real-time PCR were designed using Primer 3 software (http://fokker.wi.mit.edu/cgi-bin/primer3/primer3_www.cgi). Data were analysed using the 2^{ΔCt} method.

CD95L
forward 5′-acc ccc act caa ggt cca t-3′
reverse 5′-cga agt aca acc cag ttt cgt-3′
CXCL10
forward 5′-ggt ctg agt ggg act caa gg-3′
reverse 5′-gtg gca atg atc tca aca cg-3′
CCL6
forward 5′-gct ggc ctc ata caa gaa atg g-3′
reverse 5′-gct tag gca cct ctg aac tct c-3′
II1-β
forward 5′-cag gct ccg aga tga aca ac-3′
reverse 5′-ggt gga gag ctt tca gct cat a-3′
II-6
forward 5′-gcc tcc ttg gga ctg atg ct-3′
reverse 5′-agt ctc ctc tcc gga ctt gtg-3′
Stat-3
forward 5′-cca ctg cac tga aag gct aa-3′
reverse 5′-ata gtg agc ccc tgg aac tg-3′
CXCL2
forward 5′-caa cca cca ggc tac agg-3′
reverse 5′-gcg tca cac tca agc tct g-3′

4.21 Microarray analysis

.Cel files were generated using Affymetrix software and imported into Chipinspector. The data were analyzed by Genomatix Chipinspector as described by the manufacturer's guidelines (Genomatix GmbH, Munich, Germany, http://www.genomatix.de). dChip software was used for hierarchical clustering of datasets (http://biosuntharvard.edu/complab/dchip/). A 5% p-value was applied as a cut-off.

Gene expression profiling was performed for 3 different datasets: (1) genetic depletion of CD95L in the myeloid cell lineage (CD95L^{f/f;LysMcre}) and the control littermates (CD95L^{f/f}) and (2) mice treated with a neutralizing agent to CD95L (CD95-RB69) and vehicle-treated animals and (3) complete deletion of CD95L (CD95L^{˜}1 and wt control mice. For the dataset 1 selected genes of apoptosis and immune response from gene-ontology categories were clustered using hierarchical clustering and a sub-tree, showing similar gene expression pattern, was selected and shown in FIG. 2b. Gene ontology study was performed using EASE. For each gene ontology category, a fisher's exact p-value was calculated and adjusted using bonferroni method.

A 5% p-value was applied as a cut-off.

### LIST OF REFERENCES

1. Ackery, A., Robins, S., and Fehlings, M. G. (2006). Inhibition of Fas-mediated apoptosis through administration of soluble Fas receptor improves functional outcome and reduces posttraumatic axonal degeneration after acute spinal cord injury. J. Neurotrauma 23, 604-616.
2. Akuzawa, S., Kazui, T., Shi, E., Yamashita, K., Bashar, A. H., and Terada, H. (2008). Interleukin-1 receptor antagonist attenuates the severity of spinal cord ischemic injury in rabbits. J. Vasc. Surg.
3. Ali, K., Bilancio, A., Thomas, M., Pearce, W., Gilfillan, A. M., Tkaczyk, C., Kuehn, N., Gray, A., Giddings, J., Peskett, E., Fox, R., Bruce,l., Walker, C., Sawyer, C., Okkenhaug, K., Finan, P., and Vanhaesebroeck, B. (2004). Essential role for the p110delta phosphoinositide 3-kinase in the allergic response. Nature 431, 1007-1011.
4. Allison, J., Georgiou, H. M., Strasser, A., and Vaux, D. L. (1997). Transgenic expression of CD95 ligand on islet beta cells induces a granulocytic infiltration but does not confer immune privilege upon islet allografts. Proc. Natl. Acad. Sci. U. S. A 94, 3943-3947.
5. Altemeier, W. A., Zhu, X., Berrington, W. R., Harlan, J. M., and Liles, W. C. (2007). Fas (CD95) induces macrophage proinflammatory chemokine production via a MyD88-dependent, caspase-independent pathway. J. Leukoc. Biol. 82, 721-728.
6. Atkinson, E. A., Ostergaard, H., Kane, K., Pinkoski, M. J., Caputo, A., Olszowy, M. W., and Bleackley, R. C. (1996). A physical interaction between the cell death protein Fas and the tyrosine kinase p59fynT. J. Biol. Chem. 271, 5968-5971.
7. Austin, J. W. and Fehlings, M. G. (2008). Molecular mechanisms of Fas-mediated cell death in oligodendrocytes. J. Neurotrauma 25, 411-426.
8. Basso, D. M., Fisher, L. C., Anderson, A. J., Jakeman, L. B., McTigue, D. M., and Popovich, P. G. (2006). Basso Mouse Scale for locomotion detects differences in recovery after spinal cord injury in five common mouse strains. J. Neurotrauma 23, 635-659.
9. Baud, V. and Karin, M. (2001). Signal transduction by tumor necrosis factor and its relatives. Trends Cell Biol. 11, 372-377.
10. Boulven, I., Levasseur, S., Marois, S., Pare, G., Rollet-Labelle, E., and Naccache, P. H. (2006). Class IA phosphatidylinositide 3-kinases, rather than p110 gamma, regulate formylmethionyl-leucyl-phenylalanine-stimulated chemotaxis and superoxide production in differentiated neutrophil-like PLB-985 cells. J. Immunol. 176, 7621-7627.
11. Brown, S. B. and Savill, J. (1999). Phagocytosis triggers macrophage release of Fas ligand and induces apoptosis of bystander leukocytes. J. Immunol. 162, 480-485.
12. Casha, S., Yu, W. R., and Fehlings, M. G. (2001). Oligodendroglial apoptosis occurs along degenerating axons and is associated with FAS and p75 expression following spinal cord injury in the rat. Neuroscience 103, 203-218.
13. Casha, S., Yu, W. R., and Fehlings, M. G. (2005). FAS deficiency reduces apoptosis, spares axons and improves function after spinal cord injury. Exp. Neurol. 196, 390-400.
14. Daigle, I., Yousefi, S., Colonna, M., Green, D. R., and Simon, H. U. (2002). Death receptors bind SHP-1 and block cytokine-induced anti-apoptotic signaling in neutrophils. Nat. Med. 8, 61-67.
15. Demjen, D., Klussmann, S., Kleber, S., Zuliani, C., Stieltjes, B., Metzger, C., Hirt, U. A., Walczak, H., Falk, W., Essig, M., Edler, L., Krammer, P. H., and Martin-Villalba, A. (2004). Neutralization of CD95 ligand promotes regeneration and functional recovery after spinal cord injury. Nat. Med. 10, 389-395.
16. Desbarats, J., Birge, R. B., Mimouni-Rongy, M., Weinstein, D. E., Palerme, J. S., and Newell, M. K. (2003). Fas engagement induces neurite growth through ERK activation and p35 upregulation. Nat. Cell Biol. 5, 118-125.
17. Dhein, J., Walczak, H., Baumler, C., Debatin, K. M., and Krammer, P. H. (1995). Autocrine T-cell suicide mediated by APO-1/(Fas/CD95). Nature 373, 438-441.
18. Fecho, K. and Cohen, P. L. (1998). Fas ligand (gld)- and Fas (lpr)-deficient mice do not show alterations in the extravasation or apoptosis of inflammatory neutrophils. J. Leukoc. Biol. 64, 373-383.
19. Frommhold, D., Mannigel, I., Schymeinsky, J., Mocsai, A., Poeschl, J., Walzog, B., and Sperandio, M. (2007). Spleen tyrosine kinase Syk is critical for sustained leukocyte adhesion during inflammation in vivo. BMC. Immunol. 8, 31.
20. Gonzalez, R., Hickey, M. J., Espinosa, J. M., Nistor, G., Lane, T. E., and Keirstead, H. S. (2007). Therapeutic neutralization of CXCL10 decreases secondary degeneration and functional deficit after spinal cord injury in mice. Regen. Med. 2, 771-783.
21. Griffith, T. S., Brunner, T., Fletcher, S. M., Green, D. R., and Ferguson, T. A. (1995). Fas ligandinduced apoptosis as a mechanism of immune privilege. Science 270, 1189-1192.
22. Griffith, T. S., Yu, X., Herndon, J. M., Green, D. R., and Ferguson, T. A. (1996). CD95-induced apoptosis of lymphocytes in an immune privileged site induces immunological tolerance. Immunity. 5, 7-16.
23. Gross, O., Poeck, H., Bscheider, M., Dostert, C., Hannesschlager, N., Endres, S., Hartmann, G., Tardivel, A., Schweighoffer, E., Tybulewicz, V., Mocsai, A., Tschopp, J., and Ruland, J. (2009). Syk kinase signalling couples to the NIrp3 inflammasome for anti-fungal host defence. Nature.
24. Hahne, M., Rimoldi, D., Schroter, M., Romero, P., Schreier, M., French, L. E., Schneider, P., Bornand, T., Fontana, A., Lienard, D., Cerottini, J., and Tschopp, J. (1996). Melanoma cell expression of Fas(Apo-1/CD95) ligand: implications for tumor immune escape. Science 274, 1363-1366.
25. Hoang, T. R., Hammermuller, A., Mix, E., Kreutzer, H. J., Goerlich, R., Kohler, H., Nizze, H., Thiesen, H. J., and Ibrahim, S. M. (2004). A proinflammatory role for Fas in joints of mice with collagen-induced arthritis. Arthritis Res. Ther. 6, R404-R414.
26. Hohlbaum, A. M., Gregory, M. S., Ju, S. T., and Marshak-Rothstein, A. (2001). Fas ligand engagement of resident peritoneal macrophages in vivo induces apoptosis and the production of neutrophil chemotactic factors. J. Immunol. 167, 6217-6224.
27. Jones, L. L. and Tuszynski, M. H. (2002). Spinal cord injury elicits expression of keratan sulfate proteoglycans by macrophages, reactive microglia, and oligodendrocyte progenitors. J. Neurosci. 22, 4611-4624.
28. Kang, S. M., Hoffmann, A., Le, D., Springer, M. L., Stock, P. G., and Blau, H. M. (1997). Immune response and myoblasts that express Fas ligand. Science 278, 1322-1324.
29. Karray, S., Kress, C., Cuvellier, S., Hue-Beauvais, C., Damotte, D., Babinet, C., and Levi-Strauss, M. (2004). Complete loss of Fas ligand gene causes massive lymphoproliferation and early death, indicating a residual activity of gld allele. J. Immunol. 172, 2118-2125.
30. Kennedy, N. J., Kataoka, T., Tschopp, J., and Budd, R. C. (1999). Caspase activation is required for T cell proliferation. J. Exp. Med. 190, 1891-1896.
31. Klas, C., Debatin, K. M., Jonker, R. R., and Krammer, P. H. (1993). Activation interferes with the APO-1 pathway in mature human T cells. Int. Immunol. 5, 625-630.
32. Kleber, S., Sancho-Martinez, I., Wiestler, B., Beisel, A., Gieffers, C., Hill, O., Thiemann, M., Mueller, W., Sykora, J., Kuhn, A., Schreglmann, N., Letellier, E., Zuliani, C., Klussmann, S., Teodorczyk, M., Grone, H. J., Ganten, T. M., Sultmann, H., Tuttenberg, J., von, D. A., Regnier-Vigouroux, A., Herold-Mende, C., and Martin-Villalba, A. (2008). Yes and PI3K bind CD95 to signal invasion of glioblastoma. Cancer Cell 13, 235-248.
33. Koziol, J. A., Maxwell, D. A., Fukushima, M., Colmerauer, M. E., and Pilch, Y. H. (1981). A distribution-free test for tumor-growth curve analyses with application to an animal tumor immunotherapy experiment. Biometrics 37, 383-390.
34. Krammer, P. H. (2000). CD95's deadly mission in the immune system. Nature 407, 789-795.
35. Li, G. L., Farooque, M., and Olsson, Y. (2000). Changes of Fas and Fas ligand immunoreactivity after compression trauma to rat spinal cord. Acta Neuropathol. 100, 75-81.
36. Ma, Y., Liu, H., Tu-Rapp, H., Thiesen, H. J., Ibrahim, S. M., Cole, S. M., and Pope, R. M. (2004). Fas ligation on macrophages enhances IL-1R1-Toll-like receptor 4 signaling and promotes chronic inflammation. Nat. Immunol. 5, 380-387.
37. Matsushita, K., Wu, Y., Qiu, J., Lang-Lazdunski, L., Hirt, L., Waeber, C., Hyman, B. T., Yuan, J., and Moskowitz, M. A. (2000). Fas receptor and neuronal cell death after spinal cord ischemia. J. Neurosci. 20, 6879-6887.
38. Nagata, S. (1999). Fas ligand-induced apoptosis. Annu. Rev. Genet. 33, 29-55.
39. O'Connell, J., O'Sullivan, G. C., Collins, J. K., and Shanahan, F. (1996). The Fas counterattack: Fas-mediated T cell killing by colon cancer cells expressing Fas ligand. J. Exp. Med. 184, 1075-1082.
40. Okada, S., Nakamura, M., Mikami, Y., Shimazaki, T., Mihara, M., Ohsugi, Y., Iwamoto, Y., Yoshizaki, K., Kishimoto, T., Toyama, Y., and Okano, H. (2004). Blockade of interleukin-6 receptor suppresses reactive astrogliosis and ameliorates functional recovery in experimental spinal cord injury. J. Neurosci. Res. 76, 265-276.
41. Park, D. R., Thomsen, A. R., Frevert, C. W., Pham, U., Skerrett, S. J., Kiener, P. A., and Liles, W. C. (2003). Fas (CD95) induces proinflammatory cytokine responses by human monocytes and monocyte-derived macrophages. J. Immunol. 170, 6209-6216.
42. Peter, M. E. and Krammer, P. H. (2003). The CD95(APO-1/Fas) DISC and beyond. Cell Death. Differ. 10, 26-35.
43. Pine, P. R., Chang, B., Schoettler, N., Banquerigo, M. L., Wang, S., Lau, A., Zhao, F., Grossbard, E. B., Payan, D. G., and Brahn, E. (2007). Inflammation and bone erosion are suppressed in models of rheumatoid arthritis following treatment with a novel Syk inhibitor. Clin. Immunol. 124, 244-257.
44. Plemel, J. R., Duncan, G., Chen, K. W., Shannon, C., Park, S., Sparling, J. S., and Tetzlaff, W. (2008). A graded forceps crush spinal cord injury model in mice. J. Neurotrauma 25, 350-370.
45. Rolls, A., Shechter, R., and Schwartz, M. (2009). The bright side of the glial scar in CNS repair. Nat. Rev. Neurosci. 10, 235-241.
46. Sabelko, K. A., Kelly, K. A., Nahm, M. H., Cross, A. H., and Russell, J. H. (1997). Fas and Fas ligand enhance the pathogenesis of experimental allergic encephalomyelitis, but are not essential for immune privilege in the central nervous system. J. Immunol. 159, 3096-3099.
47. Sakurai, M., Hayashi, T., Abe, K., Sadahiro, M., and Tabayashi, K. (1998). Delayed selective motor neuron death and fas antigen induction after spinal cord ischemia in rabbits. Brain Res. 797, 23-28.
48. Sancho-Martinez, I. and Martin-Villalba, A. (2009). Tyrosine phosphorylation and CD95: a FAScinating switch. Cell Cycle 8, 838-842.
49. Schnell, L., Fearn, S., Schwab, M. E., Perry, V. H., and Anthony, D. C. (1999). Cytokine-induced acute inflammation in the brain and spinal cord. J. Neuropathol. Exp. Neurol. 58, 245-254.
50. Schymeinsky, J., Sindrilaru, A., Frommhold, D., Sperandio, M., Gerstl, R., Then, C., Mocsai, A., Scharffetter-Kochanek, K., and Walzog, B. (2006). The Vav binding site of the non-receptor tyrosine kinase Syk at Tyr 348 is critical for beta2 integrin (CD11/CD18)-mediated neutrophil migration. Blood 108, 3919-3927.
51. Schymeinsky, J., Then, C., Sindrilaru, A., Gerstl, R., Jakus, Z., Tybulewicz, V. L., Scharffetter-Kochanek, K., and Walzog, B. (2007). Syk-mediated translocation of PI3 Kdelta to the leading edge controls lamellipodium formation and migration of leukocytes. PLoS. ONE. 2, e1132.
52. Seino, K., Kayagaki, N., Okumura, K., and Yagita, H. (1997). Antitumor effect of locally produced CD95 ligand. Nat. Med. 3, 165-170.
53. Serrao, K. L., Fortenberry, J. D., Owens, M. L., Harris, F. L., and Brown, L. A. (2001). Neutrophils induce apoptosis of lung epithelial cells via release of soluble Fas ligand. Am. J. Physiol Lung Cell Mol. Physiol 280, L298-L305.
54. Shimizu, M., Yoshimoto, T., Sato, M., Morimoto, J., Matsuzawa, A., and Takeda, Y. (2005). Roles of CXC chemokines and macrophages in the recruitment of inflammatory cells and tumor rejection induced by Fas/Apo-1 (CD95) ligand-expressing tumor. Int. J. Cancer 114, 926-935.
55. Stirling, D. P., Liu, S., Kubes, P., and Yong, V. W. (2009). Depletion of Ly6G/Gr-1 leukocytes after spinal cord injury in mice alters wound healing and worsens neurological outcome. J. Neurosci. 29, 753-764.
56. Stirling, D. P. and Yong, V. W. (2008). Dynamics of the inflammatory response after murine spinal cord injury revealed by flow cytometry. J. Neurosci. Res. 86, 1944-1958.
57. Strand, S., Hofmann, W. J., Hug, H., Muller, M., Otto, G., Strand, D., Mariani, S. M., Stremmel, W., Krammer, P. H., and Galle, P. R. (1996). Lymphocyte apoptosis induced by CD95 (APO-1/Fas) ligand-expressing tumor cells—a mechanism of immune evasion? Nat. Med. 2, 1361-1366.
58. Tamir, I. and Cambier, J. C. (1998). Antigen receptor signaling: integration of protein tyrosine kinase functions. Oncogene 17, 1353-1364.
59. Trivedi, A., Olivas, A. D., and Noble-Haeusslein, L. J. (2006). Inflammation and Spinal Cord Injury: Infiltrating Leukocytes as Determinants of Injury and Repair Processes. Clin. Neurosci. Res. 6, 283-292.
60. Ugolini, G., Raoul, C., Ferri, A., Haenggeli, C., Yamamoto, Y., Salaun, D., Henderson, C. E., Kato, A. C., Pettmann, B., and Hueber, A. O. (2003). Fas/tumor necrosis factor receptor death signaling is required for axotomy-induced death of motoneurons in vivo. J. Neurosci. 23, 8526-8531.
61. Weinblatt, M. E., Kavanaugh, A., Burgos-Vargas, R., Dikranian, A. H., Medrano-Ramirez, G., Morales-Torres, J. L., Murphy, F. T., Musser, T. K., Straniero, N., Vicente-Gonzales, A. V., and Grossbard, E. (2008). Treatment of rheumatoid arthritis with a Syk kinase inhibitor: a twelveweek, randomized, placebo-controlled trial. Arthritis Rheum. 58, 3309-3318.
62. Yang, L., Blumbergs, P. C., Jones, N. R., Manavis, J., Sarvestani, G. T., and Ghabriel, M. N. (2004). Early expression and cellular localization of proinflammatory cytokines interleukin-1 beta, interleukin-6, and tumor necrosis factor-alpha in human traumatic spinal cord injury. Spine 29, 966-971.
63. Yoshino, O., Matsuno, H., Nakamura, H., Yudoh, K., Abe, Y., Sawai, T., Uzuki, M., Yonehara, S., and Kimura, T. (2004). The role of Fas-mediated apoptosis after traumatic spinal cord injury. Spine 29, 1394-1404.
64. Zhu, D., Hattori, H., Jo, H., Jia, Y., Subramanian, K. K., Loison, F., You, J., Le, Y., Honczarenko, M., Silberstein, L., and Luo, H. R. (2006). Deactivation of phosphatidylinositol 3,4,5-trisphosphate/Akt signaling mediates neutrophil spontaneous death. Proc. Natl. Acad. Sci. U. S. A 103, 14836-14841.
65. Zuliani, C., Kleber, S., Klussmann, S., Wenger, T., Kenzelmann, M., Schreglmann, N., Martinez, A., del Rio, J. A., Soriano, E., Vodrazka, P., Kuner, R., Groene, H. J., Herr, I., Krammer, P. H., and Martin-Villalba, A. (2006). Control of neuronal branching by the death receptor CD95 (Fas/Apo-1). Cell Death. Differ. 13, 31-40.
66. Zurita, M., Vaquero, J., and Zurita, I. (2001). Presence and significance of CD-95 (Fas/APO1) expression after spinal cord injury. J. Neurosurg. 94, 257-264.

1. Use of an inhibitor of the CD95/CD95L system for the prevention and/or treatment of an inflammatory disorder.

2. Use of an inhibitor of the CD95/CD95L system for the prevention and/or treatment of an inflammatory process in a neuronal disorder, particularly CNS disorder.

3. The use of claim 1, wherein the inhibitor is an antibody directed against CD95L or an antigen-binding fragment thereof.

4. The use of claim 1, wherein the inhibitor is a soluble CD95 molecule optionally fused to a heterologous polypeptide domain.

5. The use of claim 1, wherein the inflammatory disorder is a chronic inflammatory bowel disease, a rheumatoid disease, an inflammatory collagenosis or an inflammatory vasculitidis.

6. The use of claim 2, wherein the inhibitor is administered systematically.

7. The use of claim 1 in human medicine.

8. A method of treating an inflammatory disorder in a patient in need of such treatment, comprising administering to the patient an anti-inflamatory effective amount of an inhibitor of the CD95/CD95L system.


SEQUENCE LISTING (18 sequences)

SEQ ID NO: 1 - protein, 27 aa, Artificial Sequence
  T4-Fibritin
GYIPEAPRDGQAYVRKDGEWVLLSTFL

SEQ ID NO: 2 - protein, 26 aa, Artificial Sequence
  RB69-Fibritin
GYIEDAPSDGKFYVRKDGAWVELPTA

SEQ ID NO: 3 - protein, 223 aa, Artificial Sequence
  CD95-RB69 fusion protein
MVGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSSGSSGSSGSGYIEDAPSDGKFYVRKDGAWVELPTASGPSSSSSSAWSHPQFEK

SEQ ID NO: 4 - DNA, 19 bp, Artificial Sequence
  Primer CD95L forward
acccccactcaaggtccat

SEQ ID NO: 5 - DNA, 21 bp, Artificial Sequence
  Primer CD95L reverse
cgaagtacaacccagtttcgt

SEQ ID NO: 6 - DNA, 20 bp, Artificial Sequence
  Primer CXCL10 forward
ggtctgagtgggactcaagg

SEQ ID NO: 7 - DNA, 20 bp, Artificial Sequence
  Primer CXCL10 reverse
gtggcaatgatctcaacacg

SEQ ID NO: 8 - DNA, 22 bp, Artificial Sequence
  Primer CCL6 forward
gctggcctcatacaagaaatgg

SEQ ID NO: 9 - DNA, 22 bp, Artificial Sequence
  Primer CCL6 reverse
gcttaggcacctctgaactctc

SEQ ID NO: 10 - DNA, 20 bp, Artificial Sequence
  Primer II1-beta forward
caggctccgagatgaacaac

SEQ ID NO: 11 - DNA, 22 bp, Artificial Sequence
  Primer II1-beta reverse
ggtggagagctttcagctcata

SEQ ID NO: 12 - DNA, 20 bp, Artificial Sequence
  Primer II-6 forward
gcctccttgggactgatgct

SEQ ID NO: 13 - DNA, 21 bp, Artificial Sequence
  Primer II-6 reverse
agtctcctctccggacttgtg

SEQ ID NO: 14 - DNA, 20 bp, Artificial Sequence
  Primer Stat-3 forward
ccactgcactgaaaggctaa

SEQ ID NO: 15 - DNA, 20 bp, Artificial Sequence
  Primer Stat-3 reverse
atagtgagcccctggaactg

SEQ ID NO: 16 - DNA, 18 bp, Artificial Sequence
  Primer CXCL2 forward
caaccaccaggctacagg

SEQ ID NO: 17 - DNA, 19 bp, Artificial Sequence
  Primer CXCL2 reverse
gcgtcacactcaagctctg

SEQ ID NO: 18 - protein, 4 aa, Artificial Sequence
  motif in CD95
YXXL